SEQUENCE LISTING (450 sequences)

SEQ ID NO: 1 - protein, 18 aa, synthetic construct
KFERQKILDVKKDQRFFE

SEQ ID NO: 2 - protein, 16 aa, synthetic construct
MGGDDDWTHLSSKEVD

SEQ ID NO: 3 - protein, 15 aa, synthetic construct
GGDDDWTHLSSKEVD

SEQ ID NO: 4 - protein, 16 aa, synthetic construct
MASSEDYIIILPECFD

SEQ ID NO: 5 - protein, 15 aa, synthetic construct
ASSEDYIIILPECFD

SEQ ID NO: 6 - protein, 9 aa, synthetic construct
LDPETGEYL

SEQ ID NO: 7 - protein, 12 aa, synthetic construct
TSFAEYWNLLSP

SEQ ID NO: 8 - protein, 16 aa, synthetic construct
VKKDQAEPLHRKFERQ

SEQ ID NO: 9 - protein, 40 aa, synthetic construct
SHGFPPEVEEQDDGTLPMSAAQESGMDRHPAACASARINV

SEQ ID NO: 10 - protein, 176 aa, synthetic construct
RLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 11 - protein, 213 aa, synthetic construct
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 12 - protein, 209 aa, synthetic construct
GSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

SEQ ID NO: 13 - protein, 7 aa, synthetic construct
ALAPYIP

SEQ ID NO: 14 - protein, 10 aa, synthetic construct
DRHDSGLDSM

SEQ ID NO: 15 - protein, 64 aa, synthetic construct
SGTVSCPICMDGYSEIVQNGRLIVSTECGHVFCSQCLRDSLKNANTCPTCRKKINHKRYHPIYI

SEQ ID NO: 16 - protein, 76 aa, synthetic construct
MQIFVKTLTGKTITLEVEPSDTIENVKAKIQDKEGIPPDQQRLIFAGKQLEDGRTLSDYNIQRESTLHLVLRLRGG

SEQ ID NO: 17 - protein, 14 aa, synthetic construct
KFERQKILDQRFFE

SEQ ID NO: 18 - protein, 76 aa, synthetic construct
MASAARLTMMWEEVTCPICLDPFVEPVSIECGHSFCQECISQVGKGGGSVCPVCRQRFLLKNLRPNRQLANMVNNL

SEQ ID NO: 19 - protein, 40 aa, synthetic construct
SHGFPPEVEEQDDGTLPMSCAQESGMDRHPAACASARINV

SEQ ID NO: 20 - protein, 16 aa, synthetic construct
ACKNWFSSLSHFVIHL

SEQ ID NO: 21 - protein, 5 aa, synthetic construct
KFERQ

SEQ ID NO: 22 - protein, 7 aa, synthetic construct
GSGSGSS

SEQ ID NO: 23 - protein, 5 aa, synthetic construct
GSGSS

SEQ ID NO: 24 - skipped

SEQ ID NO: 25 - protein, 12 aa, synthetic construct
AAAYPYDVPDYA

SEQ ID NO: 26 - protein, 5 aa, synthetic construct
GSGSG

SEQ ID NO: 27 - protein, 10 aa, synthetic construct
GSGSGGSGSG

SEQ ID NO: 28 - protein, 20 aa, synthetic construct
GSGSGGSGSGGSGSGGSGSG

SEQ ID NO: 29 - protein, 10 aa, synthetic construct
LEGGGGSAAA

SEQ ID NO: 30 - skipped

SEQ ID NO: 31 - protein, 5 aa, synthetic construct
GGSGS

SEQ ID NO: 32 - protein, 5 aa, synthetic construct
GGGGS

SEQ ID NO: 33 - protein, 11 aa, synthetic construct
EQKLISEEDLS

SEQ ID NO: 34 - protein, 5 aa, synthetic construct
NYWLN

SEQ ID NO: 35 - protein, 19 aa, synthetic construct
EIRLKSNNYATNYAESVKG

SEQ ID NO: 36 - protein, 12 aa, synthetic construct
STARATPYYFDY

SEQ ID NO: 37 - protein, 17 aa, synthetic construct
KSSQSLLNSRARKNFLT

SEQ ID NO: 38 - protein, 7 aa, synthetic construct
WASTRES

SEQ ID NO: 39 - protein, 8 aa, synthetic construct
KQSYNLYT

SEQ ID NO: 40 - protein, 5 aa, synthetic construct
DYYMH

SEQ ID NO: 41 - protein, 17 aa, synthetic construct
RIDPEDGETKYAPKFQG

SEQ ID NO: 42 - protein, 11 aa, synthetic construct
IYYYGSRYVDY

SEQ ID NO: 43 - protein, 12 aa, synthetic construct
SASSSISSSYLH

SEQ ID NO: 44 - protein, 7 aa, synthetic construct
RTSNLAS

SEQ ID NO: 45 - protein, 9 aa, synthetic construct
QQGSSIPLT

SEQ ID NO: 46 - protein, 5 aa, synthetic construct
DYWMH

SEQ ID NO: 47 - protein, 17 aa, synthetic construct
RINLDGSDTIYADSVKG

SEQ ID NO: 48 - protein, 5 aa, synthetic construct
SRKSV

SEQ ID NO: 49 - protein, 14 aa, synthetic construct
TGSNTDVGAYDYVS

SEQ ID NO: 50 - protein, 7 aa, synthetic construct
DVDVRPS

SEQ ID NO: 51 - protein, 10 aa, synthetic construct
SSYTKSGTLV

SEQ ID NO: 52 - protein, 5 aa, synthetic construct
SYTLH

SEQ ID NO: 53 - protein, 17 aa, synthetic construct
WINAAFINTKYSQKFQG

SEQ ID NO: 54 - protein, 10 aa, synthetic construct
RASGSNGLDV

SEQ ID NO: 55 - protein, 11 aa, synthetic construct
QASRDITNYLN

SEQ ID NO: 56 - protein, 7 aa, synthetic construct
DASYLET

SEQ ID NO: 57 - protein, 9 aa, synthetic construct
QQYDSVPLT

SEQ ID NO: 58 - protein, 5 aa, synthetic construct
SQAMS

SEQ ID NO: 59 - protein, 17 aa, synthetic construct
ALSRTGDYTWYADSVRG

SEQ ID NO: 60 - protein, 15 aa, synthetic construct
NYYSSFGYNWAAFHI

SEQ ID NO: 61 - protein, 12 aa, synthetic construct
RASQDVNNNYLA

SEQ ID NO: 62 - protein, 7 aa, synthetic construct
GASRRAT

SEQ ID NO: 63 - protein, 10 aa, synthetic construct
QQYGGSPPYT

SEQ ID NO: 64 - protein, 5 aa, synthetic construct
DHGMH

SEQ ID NO: 65 - protein, 17 aa, synthetic construct
VIWLDGSSRFYADSVEG

SEQ ID NO: 66 - protein, 12 aa, synthetic construct
DRVASEGTAFDV

SEQ ID NO: 67 - protein, 11 aa, synthetic construct
WASQNVNHYLV

SEQ ID NO: 68 - protein, 7 aa, synthetic construct
DTSVRAA

SEQ ID NO: 69 - protein, 7 aa, synthetic construct
QHRSDWT

SEQ ID NO: 70 - protein, 5 aa, synthetic construct
NYYMH

SEQ ID NO: 71 - protein, 17 aa, synthetic construct
IINPSGGRTSYAQKFQG

SEQ ID NO: 72 - protein, 25 aa, synthetic construct
QRPSGYSGYGPSESYGNPTDDAFDV

SEQ ID NO: 73 - protein, 11 aa, synthetic construct
GGNNIGSRGVH

SEQ ID NO: 74 - protein, 7 aa, synthetic construct
DDSDRPS

SEQ ID NO: 75 - protein, 12 aa, synthetic construct
QVWDNSSDHLVV

SEQ ID NO: 76 - protein, 5 aa, synthetic construct
SYAMS

SEQ ID NO: 77 - protein, 17 aa, synthetic construct
AISGGGDRTYSADSVKG

SEQ ID NO: 78 - protein, 20 aa, synthetic construct
GGGGEMTAVTMDGTYYGMDV

SEQ ID NO: 79 - protein, 14 aa, synthetic construct
TGTSSNVGTYKFVS

SEQ ID NO: 80 - protein, 7 aa, synthetic construct
DVTKRPS

SEQ ID NO: 81 - protein, 10 aa, synthetic construct
CSYAGSYTYV

SEQ ID NO: 82 - protein, 5 aa, synthetic construct
SHGMH

SEQ ID NO: 83 - protein, 17 aa, synthetic construct
VISYDASNKSYADSVKG

SEQ ID NO: 84 - protein, 10 aa, synthetic construct
AFSSSASGGY

SEQ ID NO: 85 - protein, 16 aa, synthetic construct
RSSQSLVHSDGVTYLN

SEQ ID NO: 86 - protein, 7 aa, synthetic construct
KVSNRDS

SEQ ID NO: 87 - protein, 10 aa, synthetic construct
MQGTHWPPWT

SEQ ID NO: 88 - protein, 5 aa, synthetic construct
SYGMH

SEQ ID NO: 89 - protein, 17 aa, synthetic construct
IIYYDSSQRYYADSVKG

SEQ ID NO: 90 - protein, 17 aa, synthetic construct
DLPFHYHRSASFAPSDT

SEQ ID NO: 91 - protein, 12 aa, synthetic construct
RASQAVTNNYLA

SEQ ID NO: 92 - protein, 7 aa, synthetic construct
AASSRAT

SEQ ID NO: 93 - protein, 9 aa, synthetic construct
QQYGTSPIT

SEQ ID NO: 94 - protein, 5 aa, synthetic construct
SYRMN

SEQ ID NO: 95 - protein, 17 aa, synthetic construct
YISTSSSTIYYADSVKG

SEQ ID NO: 96 - protein, 4 aa, synthetic construct
AFDY

SEQ ID NO: 97 - protein, 12 aa, synthetic construct
RASQSVSSSYLA

SEQ ID NO: 98 - protein, 7 aa, synthetic construct
GASSRAT

SEQ ID NO: 99 - protein, 9 aa, synthetic construct
QQYGSSPFT

SEQ ID NO: 100 - protein, 5 aa, synthetic construct
TYYMS

SEQ ID NO: 101 - protein, 17 aa, synthetic construct
NIKQDGSEKYYVDSVKG

SEQ ID NO: 102 - protein, 4 aa, synthetic construct
PPGW

SEQ ID NO: 103 - protein, 16 aa, synthetic construct
KSSQSLLHSDGKTYLY

SEQ ID NO: 104 - protein, 7 aa, synthetic construct
EVSNRFS

SEQ ID NO: 105 - protein, 9 aa, synthetic construct
MQSIQLPVT

SEQ ID NO: 106 - protein, 5 aa, synthetic construct
DYYMY

SEQ ID NO: 107 - protein, 17 aa, synthetic construct
TISDGGSYTSYPDSVKG

SEQ ID NO: 108 - protein, 14 aa, synthetic construct
DYYGSSSYTSGFAY

SEQ ID NO: 109 - protein, 16 aa, synthetic construct
RSSQSIVHSNGNTYLE

SEQ ID NO: 110 - protein, 7 aa, synthetic construct
KVSNRFS

SEQ ID NO: 111 - protein, 9 aa, synthetic construct
FQGSHVPGT

SEQ ID NO: 112 - protein, 5 aa, synthetic construct
SYYMS

SEQ ID NO: 113 - protein, 17 aa, synthetic construct
TINSNGGSTYYPDTVKG

SEQ ID NO: 114 - protein, 7 aa, synthetic construct
QNYEGAY

SEQ ID NO: 115 - protein, 16 aa, synthetic construct
RSSQSIVHSNGNTYLE

SEQ ID NO: 116 - protein, 7 aa, synthetic construct
KVSNRFS

SEQ ID NO: 117 - protein, 9 aa, synthetic construct
FQSSHVPWT

SEQ ID NO: 118 - protein, 5 aa, synthetic construct
RFGMH

SEQ ID NO: 119 - protein, 17 aa, synthetic construct
YIRSGSDIIYYADSVKG

SEQ ID NO: 120 - protein, 9 aa, synthetic construct
SGTTVPFDY

SEQ ID NO: 121 - protein, 17 aa, synthetic construct
KSSQSLLNSGDQKNYLA

SEQ ID NO: 122 - protein, 7 aa, synthetic construct
FASTRAS

SEQ ID NO: 123 - protein, 9 aa, synthetic construct
QQHYSIPLT

SEQ ID NO: 124 - protein, 5 aa, synthetic construct
SGYWS

SEQ ID NO: 125 - protein, 16 aa, synthetic construct
YISYRGSTYYNPSLKS

SEQ ID NO: 126 - protein, 10 aa, synthetic construct
FDSDDYAMEY

SEQ ID NO: 127 - protein, 12 aa, synthetic construct
SASSSVSSSHLY

SEQ ID NO: 128 - protein, 7 aa, synthetic construct
STSNLAS

SEQ ID NO: 129 - protein, 9 aa, synthetic construct
HQWSSFPFT

SEQ ID NO: 130 - protein, 5 aa, synthetic construct
NYSMN

SEQ ID NO: 131 - protein, 17 aa, synthetic construct
SISSSSEYIYYADFVKG

SEQ ID NO: 132 - protein, 6 aa, synthetic construct
PGYRKA

SEQ ID NO: 133 - protein, 14 aa, synthetic construct
AGTSSDVGGYNYVS

SEQ ID NO: 134 - protein, 7 aa, synthetic construct
EDSKRPS

SEQ ID NO: 135 - protein, 10 aa, synthetic construct
SYCASKGHWL

SEQ ID NO: 136 - protein, 5 aa, synthetic construct
EYSIH

SEQ ID NO: 137 - protein, 17 aa, synthetic construct
GINPDNGGTRYNQKFKG

SEQ ID NO: 138 - skipped

SEQ ID NO: 139 - protein, 16 aa, synthetic construct
KSSQSLLNSDGKTYLN

SEQ ID NO: 140 - protein, 7 aa, synthetic construct
LVSKLDS

SEQ ID NO: 141 - protein, 9 aa, synthetic construct
WQGTHFPHT

SEQ ID NO: 142 - protein, 5 aa, synthetic construct
EYSMH

SEQ ID NO: 143 - protein, 17 aa, synthetic construct
GINPNNGGTSYNQKFKG

SEQ ID NO: 144 - skipped

SEQ ID NO: 145 - protein, 16 aa, synthetic construct
KSSQSLLHSDGKTYLN

SEQ ID NO: 146 - protein, 7 aa, synthetic construct
LVSKLDS

SEQ ID NO: 147 - protein, 9 aa, synthetic construct
WQGTHFPHT

SEQ ID NO: 148 - protein, 5 aa, synthetic construct
DTYMH

SEQ ID NO: 149 - protein, 17 aa, synthetic construct
RIDPANSNTKFDPKFQG

SEQ ID NO: 150 - protein, 11 aa, synthetic construct
FYGGSHWYFDV

SEQ ID NO: 151 - protein, 11 aa, synthetic construct
KASQDIKSYLS

SEQ ID NO: 152 - protein, 7 aa, synthetic construct
YATSLAD

SEQ ID NO: 153 - protein, 9 aa, synthetic construct
LQQGESPYT

SEQ ID NO: 154 - protein, 5 aa, synthetic construct
NYGMS

SEQ ID NO: 155 - protein, 17 aa, synthetic construct
TISSGGKYINYLDSLKG

SEQ ID NO: 156 - protein, 11 aa, synthetic construct
DYGSGWAWFAY

SEQ ID NO: 157 - protein, 12 aa, synthetic construct
TLSSQHSTYTIE

SEQ ID NO: 158 - protein, 11 aa, synthetic construct
LKKDGSHSTGD

SEQ ID NO: 159 - protein, 13 aa, synthetic construct
GVGDTIKEQFVYV

SEQ ID NO: 160 - protein, 5 aa, synthetic construct
NYGVH

SEQ ID NO: 161 - protein, 16 aa, synthetic construct
LMWAGGSTNYNSALMS

SEQ ID NO: 162 - protein, 7 aa, synthetic construct
YRTGFAY

SEQ ID NO: 163 - protein, 16 aa, synthetic construct
RSSQSIVHTIGNTYLE

SEQ ID NO: 164 - protein, 7 aa, synthetic construct
KVSNRFS

SEQ ID NO: 165 - protein, 9 aa, synthetic construct
FQGSHVPFT

SEQ ID NO: 166 - protein, 5 aa, synthetic construct
KYWMH

SEQ ID NO: 167 - protein, 17 aa, synthetic construct
EINPSNGRTNYNEKFKS

SEQ ID NO: 168 - protein, 4 aa, synthetic construct
YMDY

SEQ ID NO: 169 - protein, 16 aa, synthetic construct
KSSQSLFDRDGKTYLN

SEQ ID NO: 170 - protein, 7 aa, synthetic construct
LVSKLDS

SEQ ID NO: 171 - protein, 9 aa, synthetic construct
WQGTHFPWT

SEQ ID NO: 172 - protein, 5 aa, synthetic construct
DPYMH

SEQ ID NO: 173 - protein, 17 aa, synthetic construct
RIDPADGNTKYDPKFQG

SEQ ID NO: 174 - protein, 11 aa, synthetic construct
FYGSSHWYFDV

SEQ ID NO: 175 - protein, 11 aa, synthetic construct
KASQDIKRYLS

SEQ ID NO: 176 - protein, 7 aa, synthetic construct
YATSLAD

SEQ ID NO: 177 - protein, 9 aa, synthetic construct
LQQGESPYT

SEQ ID NO: 178 - protein, 5 aa, synthetic construct
NFGVH

SEQ ID NO: 179 - protein, 16 aa, synthetic construct
IMWAGGSTNYNSALMS

SEQ ID NO: 180 - protein, 7 aa, synthetic construct
YKTGFAY

SEQ ID NO: 181 - protein, 16 aa, synthetic construct
RSSQSIVHAIGNTYLE

SEQ ID NO: 182 - protein, 7 aa, synthetic construct
KVSNRFS

SEQ ID NO: 183 - protein, 9 aa, synthetic construct
FQGSHVPFT

SEQ ID NO: 184 - protein, 5 aa, synthetic construct
EYTIH

SEQ ID NO: 185 - protein, 17 aa, synthetic construct
WFHPENDNIKYNENFKD

SEQ ID NO: 186 - protein, 7 aa, synthetic construct
TSGYGDY

SEQ ID NO: 187 - protein, 16 aa, synthetic construct
KSSQSLLPSDGKTYLN

SEQ ID NO: 188 - protein, 7 aa, synthetic construct
LVSKLDS

SEQ ID NO: 189 - protein, 9 aa, synthetic construct
WQGTHFPPT

SEQ ID NO: 190 - protein, 5 aa, synthetic construct
DYSMH

SEQ ID NO: 191 - protein, 17 aa, synthetic construct
VISTYYGDTTYNQKFKG

SEQ ID NO: 192 - protein, 10 aa, synthetic construct
YGNFPASFSY

SEQ ID NO: 193 - protein, 16 aa, synthetic construct
RSSKSLLHSNGNTYLY

SEQ ID NO: 194 - protein, 7 aa, synthetic construct
RMSNLAS

SEQ ID NO: 195 - protein, 9 aa, synthetic construct
MQHLEYPFT

SEQ ID NO: 196 - protein, 5 aa, synthetic construct
RNGVQ

SEQ ID NO: 197 - protein, 16 aa, synthetic construct
VIWPGGSTNCNSALMS

SEQ ID NO: 198 - protein, 13 aa, synthetic construct
VGGNYVWDYNNYA

SEQ ID NO: 199 - protein, 16 aa, synthetic construct
RSSQNIVHSIGNTYLE

SEQ ID NO: 200 - protein, 7 aa, synthetic construct
KVSNRFS

SEQ ID NO: 201 - protein, 9 aa, synthetic construct
FQGSHVPYT

SEQ ID NO: 202 - protein, 123 aa, synthetic construct
QVQLQQSGGGLVQPGGSMKLSCVASGFTSSNYWLNWVRQSPERGLEWVAEIRLKSNNYATNYAESVKGRFTISRDDSKSSVYLQVNNLRAEDTGIYYCTRSTARATPYYFDYWGQGTTVTVSS

SEQ ID NO: 203 - protein, 120 aa, synthetic construct
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCTIIYYYGSRYVDYWGQGTTLTVSS

SEQ ID NO: 204 - protein, 114 aa, synthetic construct
EVQLVESGGGLVQPGGSLRISCTTSGFIFSDYWMHWVRQAPGKGLTWVSRINLDGSDTIYADSVKGRFTISRDNDKNTLYLQMNSLRVEDTAIYYCARSRKSVWGQGTMVTVSS

SEQ ID NO: 205 - protein, 119 aa, synthetic construct
QVQLVQSGAEVKKPGASVKVSCKTSGYSFTSYTLHWVRQAPGHRPEWMGWINAAFINTKYSQKFQGRITLTRDTSANIAYLELRSLTTEDTAVYYCARRASGSNGLDVWGQGTTVTVSS

SEQ ID NO: 206 - protein, 124 aa, synthetic construct
EVQLLESGGDLVQPGGSLRLSCAASGFTFSSQAMSWVRQAPGKGLEWVSALSRTGDYTWYADSVRGRFTVSRDDSKNIFYLEMNSLRAEDTAVYYCAKNYYSSFGYNWAAFHIWGQGTMVTVSS

SEQ ID NO: 207 - protein, 121 aa, synthetic construct
QVQLVESGGGVVQPGKSLRLSCAASGFTFRDHGMHWVRQAPGKGLEWVAVIWLDGSSRFYADSVEGRFTISRDNSKNTLYLQLTSLRAEDTAIYYCARDRVASEGTAFDVWGQGTMVTVSS

SEQ ID NO: 208 - protein, 134 aa, synthetic construct
QVQLVQSGAEVKKPGASVKVSCKASGYTFTNYYMHWVRQAPGQGLEWMGIINPSGGRTSYAQKFQGRASMTRDTSTSTVYMEVISLRSEDTAVYYCARQRPSGYSGYGPSESYGNPTDDAFDVWGQGTTVTVSS

SEQ ID NO: 209 - protein, 129 aa, synthetic construct
EVQLLESGGGLVQPGGSLRLSCAASGFTFSSYAMSWVRQAPGKGLEWVSAISGGGDRTYSADSVKGRFTISRDNSKNTLYLQINSLRVEDTAVYYCAQGGGGEMTAVTMDGTYYGMDVWGQGTTVTVSS

SEQ ID NO: 210 - protein, 119 aa, synthetic construct
QVQLVESGGGVVQPGMSLRLSCAASGFSFSSHGMHWVRQTPGKGLEWLAVISYDASNKSYADSVKGRFTISRDNSKKTLYLQMDSLRVEDTALYYCANAFSSSASGGYWGQGTLVTVSS

SEQ ID NO: 211 - protein, 126 aa, synthetic construct
QVQLVESGGGVVQPGRSLRLSCAASGFTFSSYGMHWVRQAPGKGLEWVAIIYYDSSQRYYADSVKGRFTISRDNSKNALYLQMNSLRAEDTALYYCARDLPFHYHRSASFAPSDTWGQGTLVTVSS

SEQ ID NO: 212 - protein, 113 aa, synthetic construct
EVQLVESGGGLVQPGGSLRLSCAASGFTFSSYRMNWVRQAPGKGLEWVSYISTSSSTIYYADSVKGRFTISRDNAKNSLYLQMNSLRAEDTAVYYCARAFDYWGQGTLVTVSS

SEQ ID NO: 213 - protein, 113 aa, synthetic construct
EVQLVESGGGLVQPGGSLRLSCAASGFTFSTYYMSWVRQAPGKGLEWVANIKQDGSEKYYVDSVKGRFTISRDNARNSLYLQMHSLRAEDTAVYYCASPPGWWGQGTLVTVSS

SEQ ID NO: 214 - protein, 123 aa, synthetic construct
EVQLVESGGGLVKPGGSLKLSCAASGFTFSDYYMYWVRQTPEKRLEWVATISDGGSYTSYPDSVKGRFTISRDNAKNNLYLQMSSLRSEDTAMYYCARDYYGSSSYTSGFAYWGQGTLVTVSS

SEQ ID NO: 215 - protein, 116 aa, synthetic construct
DVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSS

SEQ ID NO: 216 - protein, 118 aa, synthetic construct
DVQLVESGGGLVQPGGSRRLSCAASGFTFSRFGMHWVRQAPEKGLEWVAYIRSGSDIIYYADSVKGRFTISRDNPENTLFLQMTSLRSEDTAMYYCARSGTTVPFDYWGQGTSLTVSS

SEQ ID NO: 217 - protein, 118 aa, synthetic construct
EVQLQESGPSLVKPSQTLSLTCSVTGDSVTSGYWSWIRQFPGNKLDYMGYISYRGSTYYNPSLKSRISITRDTSKNQVYLQLKSVSSEDTATYYCSYFDSDDYAMEYWGQGTSVTVSS

SEQ ID NO: 218 - protein, 115 aa, synthetic construct
EVQLVVSGGGLVKPGGSMILSCAASGFTFSNYSMNWVRQAPGKGLEWVSSISSSSEYIYYADFVKGRFTISRDNAKNSLYLQMDSLRAEDTAVYYCAWPGYRKAWGRGTLVTVSS

SEQ ID NO: 219 - protein, 111 aa, synthetic construct
EVQLQQSGPELVKPGASVKISCKTSGYTFTEYSIHWVKQSHGESLEWIGGINPDNGGTRYNQKFKGKATLTVDKSSSTAYMDLRSLTSEDSAVYYCARESWGQGTTLTVSS

SEQ ID NO: 220 - protein, 111 aa, synthetic construct
EVQLQQSGPELVKPGASVKISCKTSGFTFTEYSMHWVKQSHGKSLEWIGGINPNNGGTSYNQKFKGKATLTVDKSSSTAYMELRSLTSEDSAVYYCARESWGQGTTLTVSS

SEQ ID NO: 221 - protein, 120 aa, synthetic construct
EVQLQQSGAELVKPGASVRLSCTASGFNIKDTYMHWVKQRPEQGLEWIGRIDPANSNTKFDPKFQGKATITSDTSSNTAYLQLSSLTSEDTAVYYCARFYGGSHWYFDVWGAGTTVTVSS

SEQ ID NO: 222 - protein, 120 aa, synthetic construct
EVHLVESGGDLVMPGGSLKLSCAASGFTFSNYGMSWVRQTPDKRLEWVATISSGGKYINYLDSLKGRFTISRDNAKNTLYLQMSSLKSEDTAMYYCAKDYGSGWAWFAYWGQGTLVTVSA

SEQ ID NO: 223 - protein, 115 aa, synthetic construct
QVQLKESGPGLVAPSQSLSITCTVSGFSLTNYGVHWVRQPPGKGLEWLGLMWAGGSTNYNSALMSRLSISKDNSKSQVFLKMNSLQTDDTAMYYCVIYRTGFAYWGQGTLVTVSA

SEQ ID NO: 224 - protein, 113 aa, synthetic construct
QVQLQQPGAELVKPGASVKLSCKASGYTFTKYWMHWVKQRPGQGLEWIGEINPSNGRTNYNEKFKSKATLTVDKSSSTAYMQLSSLTSEDSAVYYCARYMDYWGQGTSVTVSS

SEQ ID NO: 225 - protein, 120 aa, synthetic construct
EVQLQQSGAELVKPGASVRLSCTASGFNIKDPYMHWVRQRPKQGLEWIGRIDPADGNTKYDPKFQGKATLTADTSSNVAYLHLSSLTSEDTAVYYCARFYGSSHWYFDVWGAGTTVTVSS

SEQ ID NO: 226 - protein, 115 aa, synthetic construct
QVQLKESGPGLVAPSQSLSITCTVSGFSLTNFGVHWVRQPPGKGLEWLGIMWAGGSTNYNSALMSRLSISKDNSKSQVFLKMNSLQTDDTAMYYCVIYKTGFAYWGQGTLVTVSA

SEQ ID NO: 227 - protein, 116 aa, synthetic construct
QVQLQQSGAELVKPGTSVKLSCKASAYTFTEYTIHWIKQKSGQGLEWIGWFHPENDNIKYNENFKDKATLTADRSSSTVYMELSRLTSEDSAVYFCAGTSGYGDYWGQGTTLTVSS

SEQ ID NO: 228 - protein, 119 aa, synthetic construct
QVQLQQSGAELVRPGVSVKISCKGSGYKFTDYSMHWVKQSHTKSLEWIGVISTYYGDTTYNQKFKGKATITVDKSSSTAYMELARLTSEDSAIYYCATYGNFPASFSYWGQGTLVTVSA

SEQ ID NO: 229 - protein, 121 aa, synthetic construct
QVQLKESGPGLVAPSQSLSITCTVSGFSLNRNGVQWVRQPPGKGLEWLGVIWPGGSTNCNSALMSRLSISKDNSKSQVFLKMNSLHTDDTGIYYCARVGGNYVWDYNNYAWGQGTLVTVSA

SEQ ID NO: 230 - protein, 112 aa, synthetic construct
DIELTQSPSSLAVSAGEKVTMSCKSSQSLLNSRARKNFLTWYQQKPGQSPKLLIYWASTRESGVPDRFTGSGSGTDFTLTISSVQAEDLAVYYCKQSYNLYTFGGGTKLEIK

SEQ ID NO: 231 - protein, 108 aa, synthetic construct
EIVLTQSPTTMAASPGEKITITCSASSSISSSYLHWYQQKPGFSPKLLIYRTSNLASGVPARFSGSGSGTSYSLTIGTMEAEDVATYYCQQGSSIPLTFGSGTKLEIK

SEQ ID NO: 232 - protein, 110 aa, synthetic construct
QSALTQPASVSGSPGQSITISCTGSNTDVGAYDYVSWSQQLPGKAPKFVIFDVDVRPSGISDRFSGSKSGNTASLTISGLQAEDEADYYCSSYTKSGTLVFGGGTKVTVV

SEQ ID NO: 233 - protein, 107 aa, synthetic construct
DIQMTQSPSSLSASVGDRITITCQASRDITNYLNWYQQKPGKAPKLLIYDASYLETGVPSTFSGSGSGTHFTLTISSLQPDDFATYYCQQYDSVPLTFGGGTKVEIK

SEQ ID NO: 234 - protein, 109 aa, synthetic construct
EIVLTQSPGTLSLSPGERATLSCRASQDVNNNYLAWYQQKPGQAPRLLIYGASRRATGVPDRFSGRGSGTDFTLTINRLEPEDFAMYFCQQYGGSPPYTFGQGTKLEIK

SEQ ID NO: 235 - protein, 105 aa, synthetic construct
EIVLTQSPATLSLSPGERATLSCWASQNVNHYLVWYQQRPGQAPRLLLYDTSVRAAGIPARFIGSGSGTHFTLTISSLEPEDSAVYYCQHRSDWTFGQGTKVEIK

SEQ ID NO: 236 - protein, 109 aa, synthetic construct
SYVLTQPPSVSVAPGQTARITCGGNNIGSRGVHWYQQRPGQAPVLVVYDDSDRPSGIPERFSGSNSGDTATLTISRVEVGDEADYYCQVWDNSSDHLVVFGGGTKLTVL

SEQ ID NO: 237 - protein, 110 aa, synthetic construct
QSALTQPRSVSGSPGQSITISCTGTSSNVGTYKFVSWYQQHPGKAPKLMIYDVTKRPSGVPDRFSGSKSGNTASLTISGLQAEDEADYYCCSYAGSYTYVFGSGTKVTVL

SEQ ID NO: 238 - protein, 113 aa, synthetic construct
DVVMTQSPLSLPVTLGQPASISCRSSQSLVHSDGVTYLNWFQQRPGQSPRRLIYKVSNRDSGVPDRFSGSGSGTDFTLEISRVEAEDVGIYYCMQGTHWPPWTFGQGTKVEIK

SEQ ID NO: 239 - protein, 108 aa, synthetic construct
EIVLTQSPGTLSLSPGERATLSCRASQAVTNNYLAWYQQKPGQAPRLLVYAASSRATGIPDRFYGSGSGADFTLTISRLEPEDFAVYYCQQYGTSPITFGQGTRLEIK

SEQ ID NO: 240 - protein, 108 aa, synthetic construct
EIVLTQSPGTLSLSPGERATLSCRASQSVSSSYLAWYQQKPGQAPRLLIYGASSRATGIPDRFSGSGSGTDFTLTIIRLEPEDFAVYYCQQYGSSPFTFGQGTKVEIK

SEQ ID NO: 241 - protein, 112 aa, synthetic construct
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPVTFGGGTKVEIK

SEQ ID NO: 242 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPGTFGGGTKLEIK

SEQ ID NO: 243 - protein, 112 aa, synthetic construct
DVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTFGGGTKLEIK

SEQ ID NO: 244 - protein, 113 aa, synthetic construct
DIVMTQSPSSLAMSVGQKVTMSCKSSQSLLNSGDQKNYLAWYQQKPGQSPELLLYFASTRASGVPDRFIGSGSGTDFSLTISSVQAEDLADYFCQQHYSIPLTFGAGTKLELK

SEQ ID NO: 245 - protein, 108 aa, synthetic construct
QIVLTQSPAIMSASPGEKVTLTCSASSSVSSSHLYWYQQKPGSSPKLWIYSTSNLASGVPARFSGSGSGTSYSLTISSMEAEDAASYFCHQWSSFPFTFGSGTKLEIK

SEQ ID NO: 246 - protein, 110 aa, synthetic construct
QSVLTQPASVSGSPGQSITISCAGTSSDVGGYNYVSWYQQHPGKAPKLMIYEDSKRPSGVSNRFSGSKSGNTASLTISGLRAEDEADYYCSYCASKGHWLFGGGTKLAVL

SEQ ID NO: 247 - protein, 112 aa, synthetic construct
DVVMTQTPLTLSVTIGQPASISCKSSQSLLNSDGKTYLNWLLQRPGQSPKRLIYLVSKLDSRIPDRFTGSGSGTDFTLKISRVEAEDLGVYYCWQGTHFPHTFGSGTKLELK

SEQ ID NO: 248 - protein, 112 aa, synthetic construct
DVVMTQTPLTLSVTIGQPASISCKSSQSLLHSDGKTYLNWLLQRPGQSPKRLIYLVSKLDSRIPDRFTGSGSGTDFTLKISRVEAEDLGVYYCWQGTHFPHTFGAGTKLELK

SEQ ID NO: 249 - protein, 107 aa, synthetic construct
DIKMTQSPSSMYASLGERVTITCKASQDIKSYLSWYQHKPWKSPKALIYYATSLADGVPSRFSGSGSGQDYSLTISSLESDDTATYYCLQQGESPYTFGGGTKLEIK

SEQ ID NO: 250 - protein, 115 aa, synthetic construct
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVL

SEQ ID NO: 251 - protein, 112 aa, synthetic construct
DVLMTQTPLSLPVSLGDQASISCRSSQSIVHTIGNTYLEWYLQKPGQSPKLLIYKVSNRFSGVPDRFSGSGSGTDFTLKISRVEAEDLGVYYCFQGSHVPFTFGSGTKLEIK

SEQ ID NO: 252 - protein, 112 aa, synthetic construct
DVVMTQTPLTLSVTIGQPASISCKSSQSLFDRDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 253 - protein, 107 aa, synthetic construct
DIKMTQSPSSMYASLGERVTITCKASQDIKRYLSWYQQKPWKSPKILIYYATSLADGVPSRFSGTGSGQDYSLTISSLESDDVATYYCLQQGESPYTFGGGTKLEIK

SEQ ID NO: 254 - protein, 112 aa, synthetic construct
DVLMTQTPLSLPVSLGDQASISCRSSQSIVHAIGNTYLEWYLQKPGQSPKLLIYKVSNRFSGVPDRFSGSGSGTDFTLKISRVEAEDLGVYYCFQGSHVPFTFGSGTKLEIK

SEQ ID NO: 255 - protein, 112 aa, synthetic construct
DVVMTQIPLTLSITIGQPASISCKSSQSLLPSDGKTYLNWLLQRPGQSPKRLIYLVSKLDSGVPDRFTGSGSGTDFTLKISRVEADDLGVYYCWQGTHFPPTFGAGTKLELK

SEQ ID NO: 256 - protein, 112 aa, synthetic construct
DIVMTQAAPSIPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 257 - protein, 112 aa, synthetic construct
DVLMTQTPLSLPVSLGDQASISCRSSQNIVHSIGNTYLEWYLQKPGQSPKLLIYKVSNRFSGVPDRFSGSGSGTDFTLKISRVEAEDLGVYYCFQGSHVPYTFGGGTKLEIR

SEQ ID NO: 258 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 259 - protein, 725 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 260 - protein, 763 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNASLEEEWAQVECEVYGRGCPSGSLDESFYDWFERQLGATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 261 - protein, 763 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSASLEEEWAQVECEVYGRGCPSGSLDESFYDWFERQLGATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 262 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNAPLTNTVKATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 263 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSAPLTNTVKATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 264 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNDDTRHWGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 265 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSDDTRHWGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 266 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNEYHHYNKTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 267 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSEYHHYNKTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 268 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNQLFPLFRTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 269 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSQLFPLFRTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 270 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFNPVTGEVPPRYPLDARTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 271 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFNPVTGEVPPRYPLDARTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 272 - protein, 739 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNSVTEQGAELSNEERTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 273 - protein, 739 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSSVTEQGAELSNEERTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 274 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGQSGRGDLGLTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 275 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGQSGRGDLGLTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 276 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGASSLNIAGLSTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 277 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTGASSLNIAGLSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 278 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHKAKGPRKLGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 279 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHKAKGPRKLGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 280 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHAIYPRHTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 281 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHAIYPRHTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 282 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNRTIGPSVTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 283 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSRTIGPSVTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 284 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNLSSRLDATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 285 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSLSSRLDATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 286 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTHRPPMWSPVWPTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 287 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTHRPPMWSPVWPTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 288 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFGQSSLPRDGPNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 289 - protein, 739 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFGQSSLPRDGPNGGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 290 - protein, 414 aa, synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 291 - protein, 3142 aa, synthetic construct
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 292 - protein, 5 aa, synthetic construct
SYSMS

SEQ ID NO: 293 - protein, 17 aa, synthetic construct
VISYDGSNKYYADSVKG

SEQ ID NO: 294 - protein, 6 aa, synthetic construct
DRYFDL

SEQ ID NO: 295 - protein, 14 aa, synthetic construct
TGTSSDIGAYNYVS

SEQ ID NO: 296 - protein, 7 aa, synthetic construct
DVSNRPS

SEQ ID NO: 297 - protein, 9 aa, synthetic construct
SSFANSGPL

SEQ ID NO: 298 - protein, 13 aa, synthetic construct
SGSNSNIGSNTVN

SEQ ID NO: 299 - protein, 7 aa, synthetic construct
DDDLLAP

SEQ ID NO: 300 - protein, 11 aa, synthetic construct
ATWDDSLNGWV

SEQ ID NO: 301 - protein, 5 aa, synthetic construct
SYSMN

SEQ ID NO: 302 - protein, 18 aa, synthetic construct
YTSSSRSNTKKYADSVKG

SEQ ID NO: 303 - protein, 18 aa, synthetic construct
AGDFGELLTGEGYYGMDV

SEQ ID NO: 304 - protein, 11 aa, synthetic construct
SGDELGDKYVG

SEQ ID NO: 305 - protein, 7 aa, synthetic construct
QDAKRPS

SEQ ID NO: 306 - protein, 9 aa, synthetic construct
QAWDSGTMV

SEQ ID NO: 307 - protein, 5 aa, synthetic construct
DYYMY

SEQ ID NO: 308 - protein, 17 aa, synthetic construct
FISNGGGSTYYPDTVKG

SEQ ID NO: 309 - protein, 9 aa, synthetic construct
GRGYVWFAY

SEQ ID NO: 310 - protein, 12 aa, synthetic construct
TLSSQHSTYTIE

SEQ ID NO: 311 - protein, 11 aa, synthetic construct
LKKDGSHSTGD

SEQ ID NO: 312 - protein, 13 aa, synthetic construct
GVGDTIKEQFVYV

SEQ ID NO: 313 - protein, 5 aa, synthetic construct
NYGMN

SEQ ID NO: 314 - protein, 17 aa, synthetic construct
WINTYTGEPTYADDSKG

SEQ ID NO: 315 - protein, 7 aa, synthetic construct
RGLLFAY

SEQ ID NO: 316 - protein, 12 aa, synthetic construct
TLSSQHSTYTIE

SEQ ID NO: 317 - protein, 11 aa, synthetic construct
LKKDGSHSTGD

SEQ ID NO: 318 - protein, 13 aa, synthetic construct
GVGDTIKEQFVYV

SEQ ID NO: 319 - protein, 5 aa, synthetic construct
TYGMN

SEQ ID NO: 320 - protein, 17 aa, synthetic construct
WINTYSGVPTYADDFKG

SEQ ID NO: 321 - protein, 12 aa, synthetic construct
RRSDGYSNYFDY

SEQ ID NO: 322 - protein, 12 aa, synthetic construct
TLSRQHSTYTIE

SEQ ID NO: 323 - protein, 11 aa, synthetic construct
LKKDGSHSTGD

SEQ ID NO: 324 - protein, 13 aa, synthetic construct
GVGDTIKEQFVYV

SEQ ID NO: 325 - protein, 5 aa, synthetic construct
TYGMS

SEQ ID NO: 326 - protein, 17 aa, synthetic construct
WINTYSGVPTYVDDFKG

SEQ ID NO: 327 - protein, 10 aa, synthetic construct
GGDNYLWFAY

SEQ ID NO: 328 - protein, 12 aa, synthetic construct
TLNSQHSTYTIE

SEQ ID NO: 329 - protein, 11 aa, synthetic construct
LKKDGSHSTGD

SEQ ID NO: 330 - protein, 13 aa, synthetic construct
GVGDTIKEQFVYV

SEQ ID NO: 331 - protein, 5 aa, synthetic construct
DAWMD

SEQ ID NO: 332 - protein, 19 aa, synthetic construct
EIRSKANNHATYYAESVKG

SEQ ID NO: 333 - protein, 5 aa, synthetic construct
AGFAY

SEQ ID NO: 334 - protein, 17 aa, synthetic construct
KSSQSLLNSSNQKNYLA

SEQ ID NO: 335 - protein, 7 aa, synthetic construct
FASTRES

SEQ ID NO: 336 - protein, 9 aa, synthetic construct
QQHYSTPWT

SEQ ID NO: 337 - protein, 5 aa, synthetic construct
YYAIG

SEQ ID NO: 338 - protein, 17 aa, synthetic construct
CISATDGSTYYADSVKG

SEQ ID NO: 339 - protein, 15 aa, synthetic construct
VRAPYSDYCNGYYDY

SEQ ID NO: 340 - protein, 5 aa, synthetic construct
YYAIG

SEQ ID NO: 341 - protein, 17 aa, synthetic construct
CISSSDGSTYYADSVKG

SEQ ID NO: 342 - protein, 15 aa, synthetic construct
VRAPYSDYCNGYYDY

SEQ ID NO: 343 - protein, 5 aa, synthetic construct
DFGMH

SEQ ID NO: 344 - protein, 17 aa, synthetic construct
LIWYDGGYKYYADSVKG

SEQ ID NO: 345 - protein, 14 aa, synthetic construct
HLEYCSRTTCYLGH

SEQ ID NO: 346 - protein, 11 aa, synthetic construct
RASQGISDYLA

SEQ ID NO: 347 - protein, 7 aa, synthetic construct
AASTLQT

SEQ ID NO: 348 - protein, 9 aa, synthetic construct
QQLKTYPYT

SEQ ID NO: 349 - protein, 13 aa, synthetic construct
SGSSSNIGSNYVY

SEQ ID NO: 350 - protein, 7 aa, synthetic construct
RNNQRPS

SEQ ID NO: 351 - protein, 12 aa, synthetic construct
AAWDDSLCVALV

SEQ ID NO: 352 - protein, 5 aa, synthetic construct
NYSMN

SEQ ID NO: 353 - protein, 17 aa, synthetic construct
SISSSSEYIYYADFVKG

SEQ ID NO: 354 - protein, 6 aa, synthetic construct
PGYRKA

SEQ ID NO: 355 - protein, 14 aa, synthetic construct
AGTSSDVGGYNYVS

SEQ ID NO: 356 - protein, 7 aa, synthetic construct
EDSKRPS

SEQ ID NO: 357 - protein, 10 aa, synthetic construct
SYCASKGHWL

SEQ ID NO: 358 - protein, 5 aa, synthetic construct
TRSMN

SEQ ID NO: 359 - protein, 17 aa, synthetic construct
WINTNTGNRTYVQAFRG

SEQ ID NO: 360 - protein, 10 aa, synthetic construct
GAGGGYWFDS

SEQ ID NO: 361 - protein, 17 aa, synthetic construct
KSNQSLFYSSNNNNYLA

SEQ ID NO: 362 - protein, 7 aa, synthetic construct
WGSTRES

SEQ ID NO: 363 - protein, 9 aa, synthetic construct
HQYYHNPYT

SEQ ID NO: 364 - protein, 5 aa, synthetic construct
DFYMK

SEQ ID NO: 365 - protein, 17 aa, synthetic construct
DIDPKNGDTFYNQKFKG

SEQ ID NO: 366 - protein, 8 aa, synthetic construct
YYGYTMDY

SEQ ID NO: 367 - protein, 12 aa, synthetic construct
TASSSVTSSYLH

SEQ ID NO: 368 - protein, 7 aa, synthetic construct
STSNLAS

SEQ ID NO: 369 - protein, 9 aa, synthetic construct
HQYRRPPRT

SEQ ID NO: 370 - protein, 5 aa, synthetic construct
DYYMY

SEQ ID NO: 371 - protein, 17 aa, synthetic construct
TISDGGSYTYYPDNMKG

SEQ ID NO: 372 - protein, 4 aa, synthetic construct
DLGK

SEQ ID NO: 373 - protein, 12 aa, synthetic construct
TLSSQHSTYTIE

SEQ ID NO: 374 - protein, 11 aa, synthetic construct
LKKDGSHSTGD

SEQ ID NO: 375 - protein, 13 aa, synthetic construct
GVGDTIKEQFVYV

SEQ ID NO: 376 - protein, 5 aa, synthetic construct
ITALS

SEQ ID NO: 377 - protein, 17 aa, synthetic construct
AITGNAYGTYYADSVKG

SEQ ID NO: 378 - protein, 12 aa, synthetic construct
GIASDSSGYSAF

SEQ ID NO: 379 - protein, 11 aa, synthetic construct
RASQSVDNQFA

SEQ ID NO: 380 - protein, 7 aa, synthetic construct
DASRRAP

SEQ ID NO: 381 - protein, 9 aa, synthetic construct
QHRYTWLYT

SEQ ID NO: 382 - protein, 5 aa, synthetic construct
EYTMH

SEQ ID NO: 383 - protein, 17 aa, synthetic construct
GINPNNGGTRYNQKFKG

SEQ ID NO: 384 - protein, 6 aa, synthetic construct
LDGRDY

SEQ ID NO: 385 - protein, 17 aa, synthetic construct
KSSQSLLNSRTRKNYLA

SEQ ID NO: 386 - protein, 7 aa, synthetic construct
WASTRES

SEQ ID NO: 387 - protein, 8 aa, synthetic construct
KQSYNLLT

SEQ ID NO: 388 - protein, 115 aa, synthetic construct
QVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSS

SEQ ID NO: 389 - protein, 128 aa, synthetic construct
EVQLVESGGGLIQPGGSLRLSCAVSGFTVSSYSMNWVRQAPGKGLEWVSYTSSSRSNTKKYADSVKGRFTISRDNARNSLYLQMNSLRDEDTAVYYCARAGDFGELLTGEGYYGMDVWGQGTTVTVSS

SEQ ID NO: 390 - protein, 118 aa, synthetic construct
QVKLQESGGGLVQPGGSLKLSCAASGFTFRDYYMYWVRQTPEKRLEWVAFISNGGGSTYYPDTVKGRFTISRDNAKNTLYLQMSRLKSEDTAMYYCARGRGYVWFAYWGQGTTVTVFS

SEQ ID NO: 391 - protein, 116 aa, synthetic construct
QVKLQESGPELKKPGETVKISCKASGYTFTNYGMNWVKQAPGKGLKWMGWINTYTGEPTYADDSKGRFAFSLETSASTAYLQINNLKNEDMATYFCARRGLLFAYWGQGTTVTVSS

SEQ ID NO: 392 - protein, 121 aa, synthetic construct
SIQLVQSGPELKKPGETVRISCKASGYSFTTYGMNWVKQAPGKGLKWMGWINTYSGVPTYADDFKGRFAFSLETSASTAYLQINILKNEDTATYFCARRRSDGYSNYFDYWGQGSTLTVSS

SEQ ID NO: 393 - protein, 119 aa, synthetic construct
QIQLVQSGPELKKPGETVKISCKASGYTFTTYGMSWVKQAPGKGFEWMGWINTYSGVPTYVDDFKGRFAFSLETSASTAYLQINNLKNEDTAVYFCARGGDNYLWFAYWGQGTLVTVSS

SEQ ID NO: 394 - protein, 116 aa, synthetic construct
QVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSS

SEQ ID NO: 395 - protein, 124 aa, synthetic construct
EVQLVESGGGLVQPGGSLRLSCAASGFSLDYYAIGWFRQAPGKEREGVSCISATDGSTYYADSVKGRFTISRDNAKNTVYLQMNSLKPEDTAVYYCATVRAPYSDYCNGYYDYWGQGTQVTVSS

SEQ ID NO: 396 - protein, 124 aa, synthetic construct
EVQLVESGGGLVQPGGSLRLSCAASGFTLDYYAIGWFRQAPGKEREGVSCISSSDGSTYYADSVKGRFTISRDNAKNTVYLQMNSLKPEDTAVYYCATVRAPYSDYCNGYYDYWGQGTQVTVSS

SEQ ID NO: 397 - protein, 123 aa, synthetic construct
EVQLVESGGGVVQPGNSLRLSCAASGFRFSDFGMHWVRQAPGKGLEWLALIWYDGGYKYYADSVKGRFTISRDNSKNTMFLQMNSLRAEDTAVYYCATHLEYCSRTTCYLGHWGQGTLVTVSS

SEQ ID NO: 398 - protein, 115 aa, synthetic construct; identical to SEQ ID NO: 218

SEQ ID NO: 399 - protein, 119 aa, synthetic construct
QVQLVQSGSAFKKPGTSVKVSCKASGYTFETRSMNWVRQAPGQGLEYMGWINTNTGNRTYVQAFRGRFVFSLDTSVSTAYLQISNLKTEDTAVYYCARGAGGGYWFDSWGQGTLVTVSS

SEQ ID NO: 400 - protein, 117 aa, synthetic construct
EVQLVESGPEVKKPGATVKISCKVSGYTFTDFYMKWVQQAPGRGLEWMGDIDPKNGDTFYNQKFKGRVTMTADTSTGTAYMQLSSLTSEDTAVYFCASYYGYTMDYWGQGTTVTVAS

SEQ ID NO: 401 - protein, 113 aa, synthetic construct
QVQLQESGGGLVKPGGSLKLSCAASGFTFSDYYMYWVRQTPEKRLEWVATISDGGSYTYYPDNMKGRFTISRDNAKNNLYLQMSSLKSEDTAMYFCARDLGKWGQGTTVTVSS

SEQ ID NO: 402 - protein, 121 aa, synthetic construct
EVQLVESGGNLVQPGGSLRLSCTASGFTFSITALSWVRQAPEKGPQWVSAITGNAYGTYYADSVKGRFTISRDNAKNTLYLQMNGLRAEDTAIYYCVKGIASDSSGYSAFWGPGTLVTVSS

SEQ ID NO: 403 - protein, 115 aa, synthetic construct
QVQLVQSGAEVKKPGASVKVSCKASGYTFTEYTMHWVRQAPGRGLEWMGGINPNNGGTRYNQKFKGRVTMTRDTSIRTAYVELSRLTSDDTAVYYCASLDGRDYWGQGTLVTVSS

SEQ ID NO: 404 - protein, 110 aa, synthetic construct
QSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVLG

SEQ ID NO: 405 - protein, 111 aa, synthetic construct
QPVLTQSPSVSAAPRQRVTISVSGSNSNIGSNTVNWIQQLPGRAPELLMYDDDLLAPGVSDRFSGSRSGTSASLTISGLQSEDEADYYAATWDDSLNGWVFGGGTKVTVLS

SEQ ID NO: 406 - protein, 106 aa, synthetic construct
SYELTQPPSVSVSPGQTATITCSGDELGDKYVGWYQQKPGQSPLLVIYQDAKRPSGIPERFSGSNSGNTATLTISGTQAMDEADYYCQAWDSGTMVFGGGTRLTVL

SEQ ID NO: 407 - protein, 116 aa, synthetic construct
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 408 - protein, 116 aa, synthetic construct; identical to SEQ ID NO: 407

SEQ ID NO: 409 - protein, 116 aa, synthetic construct
QLVLTQSSSASFSLGASAKLTCTLSRQHSTYTIEWYQQQPLKPPKFVMELKKDGSHSTGDGIPDRFSGSSSGAHRYLSISNIQPEDEAIYICGVGDTIKEQFVYVFGGGTKVTVLG

SEQ ID NO: 410 - protein, 116 aa, synthetic construct
QLVLTQSSSASFSLGASAKLTCTLNSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 411 - protein, 114 aa, synthetic construct
DIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEIKR

SEQ ID NO: 412 - protein, 108 aa, synthetic construct
DIQLTQSPSFLSASVGDTVTFTCRASQGISDYLAWFQQKPGIAPKLLIYAASTLQTGVPSRFSGSGSGTEFTLTIRSLQSEDFGTYYCQQLKTYPYTFGQGTKVEIKS

SEQ ID NO: 413 - protein, 117 aa, synthetic construct
QSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 414 - protein, 120 aa, synthetic construct
QSVLTQPASVSGSPGQSITISCAGTSSDVGGYNYVSWYQQHPGKAPKLMIYEDSKRPSGVSNRFSGSKSGNTASLTISGLRAEDEADYYCSYCASKGHWLFGGGTKLAVLGAAAEQKLIS

SEQ ID NO: 415 - protein, 114 aa, synthetic construct
DIQMTQSPDSLAVSLGERATINCKSNQSLFYSSNNNNYLAWYQHKSGQPPKLLVYWGSTRESGVPDRFSGSGSGTDFTLTISSLQAEDVAIYYCHQYYHNPYTFGQGTKLEIKR

SEQ ID NO: 416 - protein, 109 aa, synthetic construct
EIVLTQSPSSLSASVGDRVTITCTASSSVTSSYLHWYQQKPGKAPKLLIYSTSNLASGVPSRFSGSGSGTDFTFTISSLQPEDIATYYCHQYRRPPRTFGGGTKLEIKR

SEQ ID NO: 417 - protein, 116 aa, synthetic construct; identical to SEQ ID NO: 407

SEQ ID NO: 418 - protein, 108 aa, synthetic construct
EIVLTQSPATLSLSPGERATLSCRASQSVDNQFAWYQQKPGQAPRLLIYDASRRAPGIPDRFSGSGSGTDFTLTISSLEPEDFAIYYCQHRYTWLYTFGQGTRLEIKR

SEQ ID NO: 419 - protein, 108 aa, synthetic construct
QSPDSLAVSLGERATINCKSSQSLLNSRTRKNYLAWYQQKPGQPPKLLIYWASTRESGVPDRFSGSGSGTDFTLTISSLQAEDVAVYYCKQSYNLLTFGGGTKLEIKR

SEQ ID NO: 420 - protein, 725 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 421 - protein, 763 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNASLEEEWAQVECEVYGRGCPSGSLDESFYDWFERQLGATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 422 - protein, 763 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSASLEEEWAQVECEVYGRGCPSGSLDESFYDWFERQLGATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 423 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNAPLTNTVKATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 424 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSAPLTNTVKATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 425 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNDDTRHWGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 426 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSDDTRHWGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 427 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNEYHHYNKTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 428 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSEYHHYNKTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 429 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNQLFPLFRTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 430 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSQLFPLFRTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 431 - protein, 742 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFNPVTGEVPPRYPLDARTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 432 - protein, 742 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFNPVTGEVPPRYPLDARTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 433 - protein, 739 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNSVTEQGAELSNEERTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 434 - protein, 739 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSSVTEQGAELSNEERTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 435 - protein, 735 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGQSGRGDLGLTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 436 - protein, 735 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGQSGRGDLGLTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 437 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGASSLNIAGLSTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 438 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTGASSLNIAGLSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 439 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHKAKGPRKLGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 440 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHKAKGPRKLGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 441 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHAIYPRHTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 442 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHAIYPRHTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 443 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNRTIGPSVTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 444 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSRTIGPSVTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 445 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNLSSRLDATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 446 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSLSSRLDATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 447 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTHRPPMWSPVWPTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 448 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTHRPPMWSPVWPTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 449 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFGQSSLPRDGPNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 450 - protein, 739 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFGQSSLPRDGPNGGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

(19) United States
(12) Patent Application Publication
(10) Pub. No.: US 2025/0235558 A1
(43) Pub. Date: Jul. 24, 2025

(54) DEGRON FUSION CONSTRUCTS

(71) Applicant: VectorY B.V., Amsterdam (NL)

(72) Inventors: Barbara Sanders, Amsterdam (NL); Wouter Pos, Amsterdam (NL); Pavlina Konstantinova, Amsterdam (NL); Sander van Deventer, Amsterdam (NL); Menno Spits, Amsterdam (NL)

(21) Appl. No.: 18/990,589

(22) Filed: Dec. 20, 2024

Related U.S. Application Data
(63) Continuation of application No. PCT/IB2023/000371, filed on Jun. 20, 2023.
(60) Provisional application No. 63/366,665, filed on Jun. 20, 2022.

(51) Int. Cl. A61K 48/00; A61K 47/68; A61P 25/28; C07K 14/005; C07K 16/18; C12N 15/86

(52) U.S. Cl. CPC A61K 48/005; A61K 47/6811; A61K 47/6843; A61K 47/6889; A61P 25/28; C07K 14/005; C07K 16/18; C12N 15/86; C07K 2317/565; C07K 2317/622; C12N 2750/14122; C12N 2750/14143

(57) ABSTRACT

Provided herein are degrons, compositions, and chimeric molecules comprising the degrons, and methods of using the degrons and chimeric molecules.

### RELATED APPLICATION

This application claims benefit to U.S. Provisional Application No. 63/366,665, filed on Jun. 20, 2022, the content of which is incorporated by reference herein in its entirety.

### SEQUENCE LISTING

The content of the electronically submitted Sequence Listing XML (Name: 200720_SL.xml; Size: 448,112 bytes; Created on Jun. 17, 2023) is incorporated by reference herein in its entirety.

### BACKGROUND

Neurological diseases, such as ALS, frontotemporal dementia, Huntington's disease, are characterized by misfolding and aggregation of an endogenous protein. These misfolded and aggregated proteins are not efficiently degraded by the protein degradation systems of the cell, leading to their accumulation and resulting in toxic gain of function and/or loss of physiological functions. Promoting the degradation of disease-causing proteins and aggregates by targeting the protein for degradation is an attractive therapeutic strategy for these proteinopathies. Traditional small molecule inhibitors face significant challenges such as poor drug selectivity, therapy resistance and most proteins remain undruggable. Therefore, there is a lack of therapies that directly control protein degradation of disease-causing proteins and/or aggregates.

RNA interference has been a popular approach for reducing the gene expression of these proteins, which can lead to a reduction in protein expression and prevention of aggregate formation. These therapies are not always efficient for diseases with accumulation of protein aggregates because RNA and protein levels are not necessarily directly correlated. Furthermore, it remains challenging to efficiently target abnormal repetitive GC-rich sequences which is commonly found in many neurodegenerative disorders. Thus, patients that have diseases associated with accumulated disease-causing proteins could benefit from a treatment that directly reduces the protein and/or aggregate levels.

Accordingly, there is a need in the art for treatments that promote the clearance of disease-causing proteins by targeted protein degradation.

### SUMMARY

Provided herein are degrons, compositions and chimeric molecules comprising the degrons, and methods of using the same. In an aspect, the chimeric molecules provided comprise a degron and a binding moiety. The degrons and chimeric molecules provided herein effectively target proteins and/or aggregates (e.g., TDP-43, huntingtin) for degradation. The degrons and chimeric molecules provided herein overcome shortcomings in the field of treating diseases caused by protein accumulation and/or aggregation (e.g., ALS, Huntington's disease).

In an aspect, provided herein is a chimeric molecule comprising a degron and a binding moiety, wherein the degron comprises an amino acid sequence selected from the group consisting of SEQ ID NOs: 1-21. In an embodiment, the amino acid sequence of the degron consists of an amino acid sequence selected from the group consisting of SEQ ID NOs: 1-21. In an embodiment, the degron consists of an amino acid sequence selected from the group consisting of SEQ ID NOs: 1-21.

In an embodiment, the degron is no more than 215 amino acids in length. In an embodiment, the degron is no more than 50 amino acids in length.

In an embodiment, the binding moiety is a small molecule, a nucleic acid, a lipid, or a polypeptide. In an embodiment, the binding moiety is a polypeptide.

In an embodiment, the polypeptide is at least 20 amino acids in length. In an embodiment, the polypeptide is at least 50 amino acids in length.

In an embodiment, the chimeric molecule comprises more than one degron.

In an embodiment, the binding moiety is an antibody, a binding entity, a scaffold protein, or an antibody mimetic. In an embodiment, the binding moiety is an antibody. In an embodiment, the antibody is an scFv, heavy chain only antibody, variable domain of new antigen receptor (VNAR), antibody fragment, antigen binding (Fab) fragment, monobody, DARPin, VHH antibody, or nanobody.

In an embodiment, the degron is linked to the N-terminus and the C-terminus of the polypeptide. In an embodiment, the degron is linked to the N-terminus of the polypeptide. In an embodiment, the degron is linked to the C-terminus of the polypeptide.

In an embodiment, the binding moiety is linked to the degron via a linker. In an embodiment, the degron is linked to the variable heavy chain region (VH) and/or variable light chain region (VL) of the antibody. In an embodiment, the degron is linked to the CDRH1, CDRH2 CDRH3, CDRL1, CDRL2, or CDRL3 of the antibody. In an embodiment, the linker is a peptide or chemical linker. In an embodiment, the linker is a peptide linker.

In an embodiment, the chimeric molecule comprises from N-terminus to C-terminus: a degron; a peptide linker; and an antibody. In an embodiment, the chimeric molecule comprises from N-terminus to C-terminus: an antibody; a peptide linker; and a degron. In an embodiment, the chimeric molecule comprises from N-terminus to C-terminus: a degron comprising an amino acid sequence selected from the group consisting of SEQ ID NOs: 1-8; a peptide linker; and an antibody. In an embodiment, the chimeric molecule comprises from N-terminus to C-terminus: an antibody; a peptide linker; and a degron comprising an amino acid sequence selected from the group consisting of SEQ ID NOs: 1-8.

In an embodiment, the peptide linker is 1-25 amino acids in length. In an embodiment, the peptide linker comprises an amino acid sequence selected from the group consisting of SEQ ID NOs: 22-33. In an embodiment, the peptide linker comprises the amino acid sequence of SEQ ID NO: 22. In an embodiment, the peptide linker comprises the amino acid sequence of SEQ ID NO: 23.

In an embodiment, the antibody is an scFv that specifically binds to human TDP-43. In an embodiment, the scFv specifically binds to human TDP-43 at an epitope comprising amino acids 65-71, 104-176, 115-118, 247, 201-211, 121-127, 213-223, 381-391, 133-139, 9-15, 409-410, 317-343, 215-222, 140-200, 181-195, 199-213, 307-321, 352-366, 389-411 or 397-411 of SEQ ID NO: 290. In an embodiment, the degron comprises an amino acid sequence selected from the group consisting of SEQ ID NOs: 1-19. In an embodiment, the scFv comprises a heavy chain variable domain and a light chain variable domain comprising the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively. In an embodiment, the heavy chain variable domain and light chain variable domain comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the polypeptide is an scFv or a nanobody that specifically binds to human huntingtin. In an embodiment, the scFv or nanobody specifically binds to human huntingtin at an epitope comprising amino acids 1-17, 18-38, 39-49, 50-64, 62-65, 67-76, 73-87, 80-89, 575-584, 585-516 of SEQ ID NO: 291. In an embodiment, the scFv comprises a heavy chain variable domain and a light chain variable domain comprising the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387. In an embodiment, the heavy chain variable domain and light chain variable domain comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415. In an embodiment, the nanobody comprises a heavy chain variable domain comprising the CDRH1, CDRH2, and CDRH3 amino acid sequences of SEQ ID NOs: 337, 338, and 339; or 340, 341, and 342; or a light chain variable domain comprising the CDRL1, CDRL2, and CDRL3 amino acid sequences of SEQ ID NOs: 298, 299, and 300; or 349, 350, and 351. In an embodiment, the heavy chain variable domain comprises the amino acid sequences of SEQ ID NOs: 395 or 396; or the light chain variable domain comprises the amino acid sequences of SEQ ID NOs: 405 or 413. In an embodiment, the degron comprises an amino acid sequence selected from the group consisting of SEQ ID NOs: 1-17 and 20.

In an embodiment, the polypeptide is an scFv that specifically binds to human polyglutamine (polyQ) protein. In an embodiment, the polypeptide is an scFv that specifically binds to human alpha-synuclein. In an embodiment, the polypeptide is an scFv that specifically binds to human tau protein. In an embodiment, the polypeptide is an scFv that specifically binds to human amyloid beta. In an embodiment, the polypeptide is an scFv that specifically binds to a human lamin protein (e.g., lamin A/C). In an embodiment, the polypeptide is an scFv that specifically binds to human phospholamban (PLN) protein.

In an aspect, provided herein is a pharmaceutical composition comprising a chimeric molecule disclosed herein and a pharmaceutically acceptable carrier.

In an aspect, provided herein is a polynucleotide encoding a chimeric molecule disclosed herein. In an embodiment, the polynucleotide is an mRNA. In an embodiment, the polynucleotide is a viral genome. In an embodiment, the polynucleotide is an AAV genome.

In an aspect, provided herein is an expression vector comprising a polynucleotide disclosed herein. In an aspect, provided herein is a host cell comprising a polynucleotide disclosed herein.

In an aspect, provided herein is a method of producing a chimeric molecule, the method comprising culturing a host cell disclosed herein under conditions such that the polynucleotide is expressed and the chimeric molecule is produced.

In an aspect, provided herein is a recombinant adeno associated virus (rAAV) comprising a capsid and a viral genome, wherein the viral genome comprises at least one inverted terminal repeat (ITR) region and a polynucleotide encoding a chimeric molecule disclosed herein.

In an embodiment, the capsid comprises a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof.

In an embodiment, the capsid protein comprises the amino acid sequence of any one of SEQ ID NOs: 258-289.

In an aspect, provided herein is a pharmaceutical composition comprising an rAAV disclosed herein, and a pharmaceutically acceptable carrier.

In an aspect, provided herein is a method for reducing the level of human TDP-43 or human huntingtin in a cell, the method comprising introducing into the cell a chimeric molecule disclosed herein, a pharmaceutical composition disclosed herein, a polynucleotide disclosed herein, an expression vector disclosed herein, or an rAAV disclosed herein.

In an aspect, provided herein is a method for reducing the level of aggregates of a protein in a cell, the method comprising introducing into the cell a chimeric molecule disclosed herein, a pharmaceutical composition disclosed herein, a polynucleotide disclosed herein, an expression vector disclosed herein, or an rAAV disclosed herein. In an embodiment, the protein is intracellular. In an embodiment, the protein is extracellular.

In an aspect, provided herein is a method for reducing the level of human TDP-43 or human huntingtin in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of aggregates of a protein in a cell, the method comprising expressing in the cell a chimeric molecule disclosed herein, a polynucleotide disclosed herein, an expression vector disclosed herein, or an rAAV disclosed herein.

In an embodiment, the cell is a muscle cell, microglia, astrocyte, neuron, or cardiomyocyte.

In an embodiment, the protein is human TDP-43, human huntingtin polyglutamine (PolyQ) protein, alpha-synuclein, tau protein, amyloid beta, lamin, phospholamban (PLN) protein.

In an aspect, provided herein is a method of treating a neurodegenerative or a neuromuscular disease or disorder, the method comprising administering to a subject in need thereof an effective amount of a chimeric molecule disclosed herein, a pharmaceutical composition disclosed herein, a polynucleotide disclosed herein, an expression vector disclosed herein, or an rAAV disclosed herein.

In an aspect, provided herein is a method of treating a neurodegenerative or a neuromuscular disease or disorder, the method comprising administering to a subject in need thereof an effective amount of a polynucleotide disclosed herein, or an expression vector disclosed herein in a delivery vehicle. In an embodiment, the delivery vehicle is a lipid nanoparticle (LNP), a vesicle, an exosome, a liposome, or a polymer.

In an embodiment, the neurodegenerative disease or disorder is selected from the group consisting of motor neuron disease (MND), amyotrophic lateral sclerosis (ALS), parkinsonism syndrome, Alzheimer's dementia, progressive supranuclear palsy (PSP), Huntington's disease, multiple system atrophy (MSA), spinocerebellar ataxia (SCA1, 2, 3, 6, 7, 17), spinal-bulbar muscular atrophy (SBMA), Dentatorubral-pallidoluysian atrophy (DRPLA), Lewy body disease, prion disease, and Friedreich's ataxia.

In an embodiment, the neuromuscular disease or disorder is selected from the group consisting of myopathy, hereditary cardiomyopathy, metabolic myopathy, distal myopathy, muscular dystrophy, congenital myopathy, spinal muscular atrophy (SMA), motor neuron disease, congenital myopathy, congenital muscular dystrophy, motor neuron disease, Duchenne muscular dystrophy, Becker muscular dystrophy, limb-girdle muscular dystrophies, myotonic dystrophy, myotubular myopathy, centronuclear myopathy, nemaline myopathy, selenoprotein N-related myopathy, Pompe disease, glycogen storage disease III, spinal muscular atrophy, amyotrophic lateral sclerosis, Charcot-Marie-Tooth disease, multiple sclerosis, myositis, polymyositis, and dermatomyositis.

In an embodiment, the rAAV is administered to the subject intravenously, intraperitoneally, subcutaneously, intramuscularly, intrathecally, intranasally, intracisternal, intracranially, or intradermally.

In an aspect, provided herein is a degron comprising an amino acid sequence selected from the group consisting of SEQ ID NOs: 1-5.

In an aspect, provided herein is a chimeric molecule comprising a degron that comprises an amino acid sequence selected from the group consisting of SEQ ID NOs: 1-5. In an embodiment, the amino acid sequence of the degron consists of an amino acid sequence selected from the group consisting of SEQ ID NOs: 1-5.

In aspect, provided herein is a polynucleotide encoding a degron or a chimeric disclosed herein. In an embodiment, the polynucleotide is an mRNA. In an embodiment, the polynucleotide is a viral genome. In an embodiment, the polynucleotide is an AAV genome. In an aspect, provided herein is an expression vector comprising the polynucleotide. In an aspect, provided herein is a host cell comprising the polynucleotide.

In an aspect, provided herein is a method of producing a degron or a chimeric molecule, the method comprising culturing a host cell disclosed herein under conditions such that the polynucleotide is expressed and the degron or chimeric molecule is produced.

In an aspect, provided herein is a recombinant adeno associated virus (rAAV) comprising a capsid and a viral genome, wherein the viral genome comprises at least one inverted terminal repeat (ITR) region and a polynucleotide encoding a degron or a chimeric disclosed herein. In an embodiment, the capsid comprises a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof. In an embodiment, the capsid protein comprises the amino acid sequence of SEQ ID NOs: 258-289.

In an aspect, provided herein is a pharmaceutical composition comprising a degron, a chimeric molecule, or an rAAV disclosed herein, and a pharmaceutically acceptable carrier.

### BRIEF DESCRIPTION OF THE DRAWINGS

FIG. 1 is a graph showing the mean level of TDP-43 aggregates in the cytoplasm of U2OS cells following transfection with TDP43-ANLS2KQ-GFP and either a TDP-43 scFv or a non-TDP-43 scFv control. Graph shows results from three independent experiments. Statistical evaluations were calculated by a student's t-test.

FIG. 2A and FIG. 2B are graphs showing the level of TDP-43 aggregates quantified from cell imaging (FIG. 2A) or phosphorylated TDP-43 aggregates analyzed by densitometry analysis of western blots (FIG. 2B) in the cytoplasm of U2OS cells following transfection with a panel of degron-TDP-43 scFv constructs (#104-124) compared to an scFv construct with no degron (#51). Graphs show results from three independent experiments. Statistical evaluations were calculated by a student's t-test.

FIG. 3A is a western blot showing the level of the chimeric degron-TDP-43 scFv construct #104 in U2OS cells with or without MG132 treatment, compared with the scFv construct with no degron, #51. FIG. 3B is a graph showing densitometry analysis of the western blot in FIG. 3A.

FIG. 4A is a graph showing the percent of cells that show expression of the TDP-43 scFv following transfection with degron-scFv constructs #104 or 122 and scFv construct #51 (with no degron) in U2OS cells treated with a proteasome inhibitor (MG132) or an autophagy inhibitor (CQ). FIG. 4B is a graph showing ubiquitin levels following transfection with degron-scFv constructs #104 or #122 and scFv construct #51 (with no degron) in U2OS cells treated with a proteasome inhibitor (MG132).

FIG. 5A and FIG. 5B are graphs showing the level of TDP-43 scFv in the cytoplasm of U2OS cells following transfection with degron-scFv construct #121 (FIG. 5A) or #122 (FIG. 5B) in U2OS cells treated with CQ, LY294002, or Wortmannin compared to untreated controls, as determined by imaging quantification (FIG. 5A) and densitometry analysis of a western blot (FIG. 5B).

FIG. 6A is a graph showing the level of Q97 HTT aggregates quantified from cell imaging in cells treated with the HTT scFv+degron constructs (#346-366) compared to the HTT scFv backbone construct without a degron (#252). FIG. 6B is a graph showing the quantification of soluble HTT exon 1 Q97 protein obtained via densitometric analyses of western blots. The membranes contained lysates from cells treated with the HTT scFv+degron constructs (#346-366) and are compared to the HTT scFv backbone construct without a degron (#252).

FIG. 7A is a graph showing the level of Q97 HTT aggregates quantified from cell imaging in cells treated with the HTT scFv+degron constructs (#391-528) compared to the HTT scFv backbone construct without a degron (#240). FIG. 7B is a graph showing the quantification of soluble HTT exon 1 Q97 protein obtained via densitometric analyses of western blots. The membranes contained lysates from cells treated with the HTT scFv+degron constructs (#391-528) and are compared to the HTT scFv backbone construct without a degron (#240).

### DETAILED DESCRIPTION

Provided herein are degrons, compositions and chimeric molecules comprising the degrons, and methods of using the same. In an aspect, the chimeric molecules provided comprise a degron and a binding moiety. The degrons and chimeric molecules provided herein effectively promote the degradation of proteins (e.g., TDP-43, huntingtin). The degrons and chimeric molecules provided herein overcome shortcomings in the field of treating diseases caused by protein accumulation and/or aggregation (e.g., ALS, Huntington's disease).

### Definitions

As used herein, the term “degron” refers to an amino acid sequence capable of causing intracellular degradation of a molecule to which it is attached (e.g., via the ubiquitin-proteasome system (UPS) or via autophagy (e.g., by chaperone-mediated autophagy (CMA)). The term “degron” does not encompass a full-length, naturally occurring polypeptide. In an embodiment, a degron is no more than 215 amino acids in length. In an embodiment, a degron is no more than 50 amino acids in length.

As used herein, the term “chimeric molecule” refers to any molecule comprising a degron and a second molecular entity that are not linked in nature. In certain embodiments, the second molecular entity is a small molecule, a nucleic acid, a carbohydrate, a lipid, or a polypeptide.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, F(ab′)2 fragments, disulfide-linked Fvs (sdFv), anti-idiotypic (anti-Id) antibodies (including, e.g., anti-anti-Id antibodies), variable domain of new antigen receptors (VNARs), antigen binding (Fab) fragments, monobodies, DARPins, VHH antibodies, and antigen-binding fragments of any of the above. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of proteins of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In an embodiment, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In an embodiment, the term “CDR” is a CDR as defined by Kabat et al., J. Biol. Chem. 252, 6609-6616 (1977) and Kabat et al., Sequences of proteins of immunological interest. (1991). In an embodiment, heavy chain CDRs and light chain CDRs of an antibody are defined using different conventions. In an embodiment, heavy chain CDRs and/or light chain CDRs are defined by performing structural analysis of an antibody and identifying residues in the variable region(s) predicted to make contact with an epitope region of a target molecule. CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FRs). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of proteins of immunological interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (a), delta (8), epsilon (8), gamma (γ), and mu (u), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an antibody), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, a “vector” refers to a nucleic acid molecule that is a vehicle for introducing a nucleic acid molecule (e.g., a polynucleotide disclosed herein) into a cell.

As used herein, an “expression vector” refers to a vector comprising transcriptional regulatory elements operably linked to a gene of interest (e.g., a polynucleotide disclosed herein) that facilitate the expression of the gene of interest in a cell and/or a cell free expression system.

As used herein, the term “transgene” refers to a non-AAV nucleic acid sequence that encodes a polypeptide (e.g., an antibody or scFv) or non-coding RNA (e.g., an miRNA, shRNA, siRNA, antisense RNA, gRNA, antagomir, miRNA sponge, RNA aptazyme, RNA ribozyme, or RNA aptamer).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a polynucleotide sequence (e.g., a transgene disclosed herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In an embodiment, the TRE is upstream from the transgene.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. When a sequence is described herein as being a certain percentage identical to a reference sequence, the percentage identity to the reference sequence is determined across the full length of the reference sequence.

As used herein, the term “effective amount” in the context of the administration of an AAV to a subject refers to the amount of the AAV that achieves a desired prophylactic or therapeutic effect.

### Degrons

In an aspect, provided herein is a degron. The amino acid sequences of exemplary degrons disclosed herein are provided in Table 1 below.

TABLE 1
Amino acid sequences of exemplary degrons

| SEQ ID NO | Degron ID | Sequence |
|---|---|---|
| 1 | 17 | KFERQKILDVKKDQRFFE |
| 2 | 18 | MGGDDDWTHLSSKEVD |
| 3 | 19 | GGDDDWTHLSSKEVD |
| 4 | 20 | MASSEDYIIILPECFD |
| 5 | 21 | ASSEDYIIILPECFD |
| 6 | 9 | LDPETGEYL |
| 7 | 10 | TSFAEYWNLLSP |
| 8 | 15 | VKKDQAEPLHRKFERQ |
| 9 | 2 | SHGFPPEVEEQDDGTLPMSAAQESGMDRHPAACASARINV |
| 10 | 4 | RLNFGDDIPSALRIAKKKRWNSIEERRIHQESELHSYLSRLI |
| | | AAERERELEECQRNHEGDEDDSHVRAQQACIEAKHDKYM |
| | | ADMDELFSQVDEKRKKRDIPDYLCGKISFELMREPCITPSGI |
| | | TYDRKDIEEHLQRVGHFDPVTRSPLTQEQLIPNLAMKEVID |
| | | AFISENGWVEDY |
| 11 | 5 | MPRRAENWDEAEVGAEEAGVEEYGPEEDGGEESGAEESG |
| | | PEESGPEELGAEEEMEAGRPRPVLRSVNSREPSQVIFCNRSP |
| | | RVVLPVWLNFDGEPQPYPTLPPGTGRRIHSYRGHLWLFRD |
| | | AGTHDGLLVNQTELFVPSLNVDGQPIFANITLPVYTLKERC |
| | | LQVVRSLVKPENYRRLDIVRSLYEDLEDHPNVQKDLERLT |
| | | QERIAHQRMGD |
| 12 | 6 | GSVNISGQNTMNMVKVPECRLADELGGLWENSRFTDCCL |
| | | CVAGQEFQAHKAILAARSPVFSAMFEHEMEESKKNRVEIN |
| | | DVEPEVFKEMMCFIYTGKAPNLDKMADDLLAAADKYALE |
| | | RLKVMCEDALCSNLSVENAAEILILADLHSADQLKTQAVD |
| | | FINYHASDVLETSGWKSMVVSHPHLVAEAYRSLASAQCPF |
| | | LGPPRKRLKQS |
| 13 | 7 | ALAPYIP |
| 14 | 8 | DRHDSGLDSM |
| 15 | 12 | SGTVSCPICMDGYSEIVQNGRLIVSTECGHVFCSQCLRDSL |
| | | KNANTCPTCRKKINHKRYHPIYI |
| 16 | 13 | MQIFVKTLTGKTITLEVEPSDTIENVKAKIQDKEGIPPDQQR |
| | | LIFAGKQLEDGRTLSDYNIQRESTLHLVLRLRGG |
| 17 | 16 | KFERQKILDQRFFE |
| 18 | 11 | MASAARLTMMWEEVTCPICLDPFVEPVSIECGHSFCQECIS |
| | | QVGKGGGSVCPVCRQRFLLKNLRPNRQLANMVNNL |
| 19 | 1 | SHGFPPEVEEQDDGTLPMSCAQESGMDRHPAACASARINV |
| 20 | 3 | ACKNWFSSLSHFVIHL |
| 21 | 14 | KFERQ |

In an embodiment, the degron comprises an amino acid sequence selected from the group consisting of SEQ ID NOs: 1-21. In an embodiment, the amino acid sequence of the degron consists of an amino acid sequence selected from the group consisting of SEQ ID NOs: 1-21. In an embodiment, the degron comprises the amino acid sequence of SEQ ID NO: 1. In an embodiment, the amino acid sequence of the degron consists of the amino acid sequence of SEQ ID NO: 1. In an embodiment, the degron comprises the amino acid sequence of SEQ ID NO: 2. In an embodiment, the amino acid sequence of the degron consists of the amino acid sequence of SEQ ID NO: 2. In an embodiment, the degron comprises the amino acid sequence of SEQ ID NO: 3. In an embodiment, the amino acid sequence of the degron consists of the amino acid sequence of SEQ ID NO: 3. In an embodiment, the degron comprises the amino acid sequence of SEQ ID NO: 4. In an embodiment, the amino acid sequence of the degron consists of the amino acid sequence of SEQ ID NO: 4. In an embodiment, the degron comprises the amino acid sequence of SEQ ID NO: 5. In an embodiment, the amino acid sequence of the degron consists of the amino acid sequence of SEQ ID NO: 5.

In an aspect, provided herein is a composition comprising a degron comprising an amino acid sequence selected from the group consisting of SEQ ID NOs: 1-21. In an embodiment, the amino acid sequence of the degron consists of an amino acid sequence selected from the group consisting of SEQ ID NOs: 1-21.

In an aspect, provided herein is a composition comprising a degron comprising the amino acid sequence of SEQ ID NO: 1. In an embodiment, the amino acid sequence of the degron consists of the amino acid sequence of SEQ ID NO: 1.

In an aspect, provided herein is a composition comprising a degron comprising the amino acid sequence of SEQ ID NO: 2. In an embodiment, the amino acid sequence of the degron consists of the amino acid sequence of SEQ ID NO: 2.

In an aspect, provided herein is a composition comprising a degron comprising the amino acid sequence of SEQ ID NO: 3. In an embodiment, the amino acid sequence of the degron consists of the amino acid sequence of SEQ ID NO: 3.

In an aspect, provided herein is a composition comprising a degron comprising the amino acid sequence of SEQ ID NO: 4. In an embodiment, the amino acid sequence of the degron consists of the amino acid sequence of SEQ ID NO: 4.

In an aspect, provided herein is a composition comprising a degron comprising the amino acid sequence of SEQ ID NO: 5. In an embodiment, the amino acid sequence of the degron consists of the amino acid sequence of SEQ ID NO: 5.

In an embodiment, the degron causes protein degradation of an intracellular protein via the ubiquitin-proteasome system (UPS). In an embodiment, the degron causes protein degradation of an intracellular protein or via autophagy (e.g., by chaperone-mediated autophagy (CMA)). In an embodiment, the degron causes protein degradation of an extracellular protein via the ubiquitin-proteasome system (UPS). In an embodiment, the degron causes protein degradation of an extracellular protein or via autophagy (e.g., by chaperone-mediated autophagy (CMA)).

### Chimeric Molecules

In an aspect, provided herein is a chimeric molecule comprising a degron and a binding moiety, wherein the degron comprises an amino acid sequence selected from the group consisting of SEQ ID NOs: 1-21. In an embodiment, the amino acid sequence of the degron consists of an amino acid sequence selected from the group consisting of SEQ ID NOs: 1-21. In an embodiment, the degron consists of an amino acid sequence selected from the group consisting of SEQ ID NOs: 1-21.

In an embodiment, the degron is no more than 215 amino acids in length, e.g., 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, or 51 amino acids in length.

In an embodiment, the degron is no more than 50 amino acids in length, e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 amino acids in length.

In an embodiment, the binding moiety is a small molecule, a nucleic acid, a lipid, or a polypeptide. In an embodiment, the binding moiety is a non-coding RNA (e.g., an miRNA, shRNA, siRNA, antisense RNA, gRNA, antagomir, miRNA sponge, RNA aptazyme, RNA ribozyme, or RNA aptamer). In an embodiment, the binding moiety is an oligonucleotide.

In an embodiment, the chimeric molecule comprises a degron comprising the amino acid sequence of SEQ ID NO: 1 and a small molecule, a nucleic acid, a lipid, or a polypeptide. In an embodiment, the chimeric molecule comprises a degron comprising the amino acid sequence of SEQ ID NO: 2 and a small molecule, a nucleic acid, a lipid, or a polypeptide. In an embodiment, the chimeric molecule comprises a degron comprising the amino acid sequence of SEQ ID NO: 3 and a small molecule, a nucleic acid, a lipid, or a polypeptide. In an embodiment, the chimeric molecule comprises a degron comprising the amino acid sequence of SEQ ID NO: 4 and a small molecule, a nucleic acid, a lipid, or a polypeptide. In an embodiment, the chimeric molecule comprises a degron comprising the amino acid sequence of SEQ ID NO: 5 and a small molecule, a nucleic acid, a lipid, or a polypeptide. In an embodiment, the chimeric molecule comprises a degron comprising the amino acid sequence of SEQ ID NO: 6 and a small molecule, a nucleic acid, a lipid, or a polypeptide. In an embodiment, the chimeric molecule comprises a degron comprising the amino acid sequence of SEQ ID NO: 7 and a small molecule, a nucleic acid, a lipid, or a polypeptide. In an embodiment, the chimeric molecule comprises a degron comprising the amino acid sequence of SEQ ID NO: 8 and a small molecule, a nucleic acid, a lipid, or a polypeptide. In an embodiment, the chimeric molecule comprises a degron comprising the amino acid sequence of SEQ ID NO: 9 and a small molecule, a nucleic acid, a lipid, or a polypeptide. In an embodiment, the chimeric molecule comprises a degron comprising the amino acid sequence of SEQ ID NO: 10 and a small molecule, a nucleic acid, a lipid, or a polypeptide. In an embodiment, the chimeric molecule comprises a degron comprising the amino acid sequence of SEQ ID NO: 11 and a small molecule, a nucleic acid, a lipid, or a polypeptide. In an embodiment, the chimeric molecule comprises a degron comprising the amino acid sequence of SEQ ID NO: 12 and a small molecule, a nucleic acid, a lipid, or a polypeptide. In an embodiment, the chimeric molecule comprises a degron comprising the amino acid sequence of SEQ ID NO: 13 and a small molecule, a nucleic acid, a lipid, or a polypeptide. In an embodiment, the chimeric molecule comprises a degron comprising the amino acid sequence of SEQ ID NO: 14 and a small molecule, a nucleic acid, a lipid, or a polypeptide. In an embodiment, the chimeric molecule comprises a degron comprising the amino acid sequence of SEQ ID NO: 15 and a small molecule, a nucleic acid, a lipid, or a polypeptide. In an embodiment, the chimeric molecule comprises a degron comprising the amino acid sequence of SEQ ID NO: 16 and a small molecule, a nucleic acid, a lipid, or a polypeptide. In an embodiment, the chimeric molecule comprises a degron comprising the amino acid sequence of SEQ ID NO: 17 and a small molecule, a nucleic acid, a lipid, or a polypeptide. In an embodiment, the chimeric molecule comprises a degron comprising the amino acid sequence of SEQ ID NO: 18 and a small molecule, a nucleic acid, a lipid, or a polypeptide. In an embodiment, the chimeric molecule comprises a degron comprising the amino acid sequence of SEQ ID NO: 19 and a small molecule, a nucleic acid, a lipid, or a polypeptide. In an embodiment, the chimeric molecule comprises a degron comprising the amino acid sequence of SEQ ID NO: 20 and a small molecule, a nucleic acid, a lipid, or a polypeptide. In an embodiment, the chimeric molecule comprises a degron comprising the amino acid sequence of SEQ ID NO: 21 and a small molecule, a nucleic acid, a lipid, or a polypeptide.

In an embodiment, the binding moiety is a polypeptide. In an embodiment, the polypeptide is at least 20 amino acids in length. In an embodiment, the polypeptide is at least 50 amino acids in length, e.g., at least 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length.

In an embodiment, the polypeptide is between 20-100, 20-200, 20-300, 20-400, 20-500, 20-600, 20-700, 20-800, 20-900, 20-1000, 20-1100, 20-1200, 20-1300, 20-1400, 20-1500, 20-1600, 20-1700, 20-1800, 20-1900, or 20-2000 amino acids in length. In an embodiment, the polypeptide is between 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, or 50-2000 amino acids in length. In an embodiment, the polypeptide is between 75-100, 75-200, 75-300, 75-400, 75-500, 75-600, 75-700, 75-800, 75-900, 75-1000, 75-1100, 75-1200, 75-1300, 75-1400, 75-1500, 75-1600, 75-1700, 75-1800, 75-1900, or 75-2000 amino acids in length. In an embodiment, the polypeptide is between 100-200, 100-300, 100-400, 100-500, 100-600, 100-700, 100-800, 100-900, 100-1000, 100-1100, 100-1200, 100-1300, 100-1400, 100-1500, 100-1600, 100-1700, 100-1800, 100-1900, or 100-2000 amino acids in length.

In an embodiment, the chimeric molecule comprises more than one degron. In an embodiment, the chimeric molecule comprises 2, 3, 4, or 5 degrons. In an embodiment, each of the degrons comprises the same amino acid sequence selected from the group consisting of SEQ

ID NOs: 1-21. In an embodiment, each of the degrons comprises a different amino acid sequence selected from the group consisting of SEQ ID NOs: 1-21.

In an embodiment, the chimeric molecule comprises a first degron and a second degron. In an embodiment, the first degron and the second degron comprise the same amino acid sequence selected from the group consisting of SEQ ID NOs: 1-21. In an embodiment, the first degron and the second degron comprise the same amino acid sequence selected from the group consisting of SEQ ID NOs: 1-21. In an embodiment, the first degron and the second degron comprise a different amino acid sequence selected from the group consisting of SEQ ID NOs: 1-21. In an embodiment, the first degron and the second degron are both linked to the N-terminus of the polypeptide. In an embodiment, the first degron and the second degron are both linked to the C-terminus of the polypeptide. In an embodiment, the first degron is linked to the N-terminus of the polypeptide and the second degron is linked to the C-terminus of the polypeptide.

In an embodiment, the binding moiety is an antibody, a binding entity, a scaffold protein, or an antibody mimetic. In an embodiment, the binding moiety is an antibody. In an embodiment, the antibody is an scFv, heavy chain only antibody, variable domain of new antigen receptor (VNAR), antibody fragment, antigen binding (Fab) fragment, monobody, DARPin, VHH antibody, or nanobody. In an embodiment, the antibody mimetic is an affibody, an adectin, an aptamer, an affimer, an affitin, an anticalin, an avimer, a fynomer, an armadillo repeat protein, or a knottin.

In an embodiment, the degron is linked to the N-terminus and the C-terminus of the polypeptide. In an embodiment, the degron is linked to the N-terminus of the polypeptide. In an embodiment, the degron is linked to the C-terminus of the polypeptide.

In an embodiment, the degron is linked to the variable heavy chain region (VH) and/or the variable light chain region (VL) of the antibody. In an embodiment, the degron is linked to the HCDR1, HCDR2 HCDR3, LCDR1, LCDR2, or LCDR3 of the antibody.

In an embodiment, the binding moiety is linked to the degron via a linker. In an embodiment, the linker is a peptide disulfide, or chemical linker. In an embodiment, the chemical linker is an imine, oxime, hydrazone, phosphoramidate, acetal-based, N-ethoxybenzylimidazole (NEBI), or maleic acid-derived linker. In an embodiment, the linker is a peptide linker. The amino acid sequences of exemplary peptide linkers are listed in Table 2 below.

TABLE 2
Amino acid sequences of exemplary peptide
linkers

| SEQ ID NO: | Amino acid sequence |
|---|---|
| 22 | GSGSGSS |
| 23 | GSGSS |
| 24 | NS |
| 25 | AAAYPYDVPDYA |
| 26 | GSGSG |
| 27 | GSGSGGSGSG |
| 28 | GSGSGGSGSGGSGSGGSGSG |
| 29 | LEGGGGSAAA |
| 30 | SGS |
| 31 | GGSGS |
| 32 | GGGGS |
| 33 | EQKLISEEDLS |

In an embodiment, the chimeric molecule comprises from N-terminus to C-terminus: a degron; a peptide linker; and an antibody.

In an embodiment, the chimeric molecule comprises from N-terminus to C-terminus: a degron comprising the amino acid sequence of SEQ ID NO: 1; a peptide linker; and an antibody that specifically binds to TDP-43, huntingtin, polyglutamine (polyQ) protein, alpha-synuclein, tau protein, amyloid beta, lamin, or phospholamban (PLN) protein. In an embodiment, the chimeric molecule comprises from N-terminus to C-terminus: a degron comprising the amino acid sequence of SEQ ID NO: 2; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 3; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 4; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 5; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 6; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 7; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 8; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 9; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 10; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 11; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 12; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 13; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 14; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 15; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 16; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 17; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 18; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 19; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 20; a peptide linker; and an antibody that specifically binds to TDP-43, huntingtin, polyQ protein, alpha-synuclein, tau protein, amyloid beta, lamin, or PLN protein. In an embodiment, the chimeric molecule comprises from N-terminus to C-terminus: a degron comprising the amino acid sequence of SEQ ID NO: 21; a peptide linker; and an antibody that specifically binds to TDP-43, huntingtin, polyQ protein, alpha-synuclein, tau protein, amyloid beta, lamin, or PLN protein.

In an embodiment, the chimeric molecule comprises from N-terminus to C-terminus: a degron comprising the amino acid sequence of SEQ ID NO: 1; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 2; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 3; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 4; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 5; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 6; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 7; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 8 a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 9; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 10; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 11; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 12; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 13; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 14; a peptide linker comprising an amino acid sequence selected from the group consisting of SEQ ID NOs: 22-33;

and an antibody. In an embodiment, the chimeric molecule comprises from N-terminus to C-terminus: a degron comprising the amino acid sequence of SEQ ID NO: 15; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 16; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 17; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 18; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 19; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 20; a peptide linker comprising an amino acid sequence selected from the group consisting of SEQ ID NOs: 22-33; and an antibody. In an embodiment, the chimeric molecule comprises from N-terminus to C-terminus: a degron comprising the amino acid sequence of SEQ ID NO: 21; a peptide linker comprising an amino acid sequence selected from the group consisting of SEQ ID NOs: 22-33; and an antibody.

In an embodiment, the chimeric molecule comprises from N-terminus to C-terminus: a degron comprising the amino acid sequence of SEQ ID NO: 1; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 2; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 3; a peptide linker comprising an amino acid sequence selected from the group consisting of SEQ ID NOs: 22-33; and an antibody hat specifically binds to TDP-43. In an embodiment, the chimeric molecule comprises from N-terminus to C-terminus: a degron comprising the amino acid sequence of SEQ ID NO: 4; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 5; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 6; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 7; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 8 a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 9; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 10; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 11; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 12; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 13; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 14; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 15; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 16; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 17; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 18; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 19; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 20; a peptide linker comprising an amino acid sequence selected from the group consisting of SEQ ID NOs: 22-33; and an antibody that specifically binds to TDP-43. In an embodiment, the chimeric molecule comprises from N-terminus to C-terminus: a degron comprising the amino acid sequence of SEQ ID NO: 21; a peptide linker comprising an amino acid sequence selected from the group consisting of SEQ ID NOs: 22-33; and an antibody that specifically binds to TDP-43.

In an embodiment, the chimeric molecule comprises from N-terminus to C-terminus: a degron comprising the amino acid sequence of SEQ ID NO: 1; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 2; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 3; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 4; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 5; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 6; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 7; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 8 a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 9; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 10; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 11; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 12; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 13; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 14; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 15; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 16; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 17; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 18; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 19; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 20; a peptide linker comprising an amino acid sequence selected from the group consisting of SEQ ID NOs: 22-33; and an antibody that specifically binds to huntingtin. In an embodiment, the chimeric molecule comprises from N-terminus to C-terminus: a degron comprising the amino acid sequence of SEQ ID NO: 21; a peptide linker comprising an amino acid sequence selected from the group consisting of SEQ ID NOs: 22-33; and an antibody that specifically binds to huntingtin.

In an embodiment, the chimeric molecule comprises from N-terminus to C-terminus: an antibody; a peptide linker; and a degron. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 1. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 2. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 3. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 4. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 5. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 6. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 7. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 8. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 9. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 10. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 11. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 12. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 13. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 14. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 15. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 16. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 17. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 18. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 19. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 20. In an embodiment, the chimeric molecule comprises from N-terminus to C-terminus: an antibody that specifically binds to TDP-43, huntingtin, polyQ protein, alpha-synuclein, tau protein, amyloid beta, lamin, or PLN protein, a peptide linker, and a degron comprising the amino acid sequence of SEQ ID NO: 21.

In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 1. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 2. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 3. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 4. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 5. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 6. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 7. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 8. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 9. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 10. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 11. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 12. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 13. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 14. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 15. In an embodiment, the chimeric molecule comprises from N-terminus to C-terminus: an antibody, a peptide linker comprising an amino acid sequence selected from the group consisting of SEQ ID

NOs: 22-33, and a degron comprising the amino acid sequence of SEQ ID NO: 16. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 17. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 18. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 19. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 20. In an embodiment, the chimeric molecule comprises from N-terminus to C-terminus: an antibody, a peptide linker comprising an amino acid sequence selected from the group consisting of SEQ ID NOs: 22-33, and a degron comprising the amino acid sequence of SEQ ID NO: 21.

In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 1. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 2. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 3. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 4. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 5. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 6. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 7. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 8. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 9. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 10. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 11. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 12. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 13. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 14. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 15. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 16. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 17. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 18. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 19. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 20. In an embodiment, the chimeric molecule comprises from N-terminus to C-terminus: an antibody that specifically binds to TDP-43, a peptide linker comprising an amino acid sequence selected from the group consisting of SEQ ID NOs: 22-33, and a degron comprising the amino acid sequence of SEQ ID NO: 21.

In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 1. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 2. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 3. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 4. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 5. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 6. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 7. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 8. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 9. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 10. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 11. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 12. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 13. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 14. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 15. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 16. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 17. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 18. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 19. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 20. In an embodiment, the chimeric molecule comprises from N-terminus to C-terminus: an antibody that specifically binds to huntingtin, a peptide linker comprising an amino acid sequence selected from the group consisting of SEQ ID NOs: 22-33, and a degron comprising the amino acid sequence of SEQ ID NO: 21.

In an embodiment, the chimeric molecule comprises from N-terminus to C-terminus: a degron comprising an amino acid sequence selected from the group consisting of SEQ ID NOs: 1-8; a peptide linker; and an antibody. In an embodiment, the chimeric molecule comprises from N-terminus to C-terminus: an antibody; a peptide linker; and a degron comprising an amino acid sequence selected from the group consisting of SEQ ID NOs: 1-8.

In an embodiment, the peptide linker is 1-25 amino acids in length. In an embodiment, the peptide linker comprises an amino acid sequence selected from the group consisting of SEQ ID NOs: 22-32. In an embodiment, the peptide linker comprises the amino acid sequence of SEQ ID NO: 22. In an embodiment, the peptide linker comprises the amino acid sequence of SEQ ID NO: 23.

In an embodiment, the antibody is an scFv that specifically binds to human TDP-43. In an embodiment, the scFv specifically binds to phosphorylated human TDP-43. In an embodiment, the scFv specifically binds to human TDP-43 at an epitope comprising amino acids 65-71, 104-176, 115-118, 247, 201-211, 121-127, 213-223, 381-391, 133-139, 9-15, 409-410, 317-343, 215-222, 140-200, 181-195, 199-213, 307-321, 352-366, 389-411, or 397-411 of SEQ ID NO: 290.

Amino acid sequence of TDP-43
(SEQ ID NO: 290)
MSEYIRVTEDENDEPIEIPSEDDGTVLLSTVTAQFPGACGLRYRNPVSQC
MRGVRLVEGILHAPDAGWGNLVYVVNYPKDNKRKMDETDASSAVKVKRAV
QKTSDLIVLGLPWKTTEQDLKEYFSTFGEVLMVQVKKDLKTGHSKGFGFV
RFTEYETQVKVMSQRHMIDGRWCDCKLPNSKQSQDEPLRSRKVFVGRCTE
DMTEDELREFFSQYGDVMDVFIPKPFRAFAFVTFADDQIAQSLCGEDLII
KGISVHISNAEPKHNSNRQLERSGRFGGNPGGFGNQGGFGNSRGGGAGLG
NNQGSNMGGGMNFGAFSINPAMMAAAQAALQSSWGMMGMLASQQNQSGPS
GNNQNQGNMQREPNQAFGSGNNSYSGSNSGAAIGWGSASNAGSGSGFNGG
FGSSMDSKSSGWGM

Amino acid sequences of exemplary TDP-43 scFvs are listed in Tables 3-5 below.

TABLE 3
Amino acid sequences of the CDRs of exemplary TDP-43 scFvs

| Construct ID | SEQ ID NO: | TDP-43 scFv | CDR | Amino acid sequence |
|---|---|---|---|---|
| 50 | 34 | E6-VH7VK9 | CDRH1 | NYWLN |
| | 35 | E6-VH7VK9 | CDRH2 | EIRLKSNNYATNYAESVKG |
| | 36 | E6-VH7VK9 | CDRH3 | STARATPYYFDY |
| | 37 | E6-VH7VK9 | CDRL1 | KSSQSLLNSRARKNFLT |
| | 38 | E6-VH7VK9 | CDRL2 | WASTRES |
| | 39 | E6-VH7VK9 | CDRL3 | KQSYNLYT |
| 51 | 40 | 3B12A | CDRH1 | DYYMH |
| | 41 | 3B12A | CDRH2 | RIDPEDGETKYAPKFQG |
| | 42 | 3B12A | CDRH3 | IYYYGSRYVDY |
| | 43 | 3B12A | CDRL1 | SASSSISSSYLH |
| | 44 | 3B12A | CDRL2 | RTSNLAS |
| | 45 | 3B12A | CDRL3 | QQGSSIPLT |
| 52 | 46 | NI-205-51C1 | CDRH1 | DYWMH |
| | 47 | NI-205-51C1 | CDRH2 | RINLDGSDTIYADSVKG |
| | 48 | NI-205-51C1 | CDRH3 | SRKSV |
| | 49 | NI-205-51C1 | CDRL1 | TGSNTDVGAYDYVS |
| | 50 | NI-205-51C1 | CDRL2 | DVDVRPS |
| | 51 | NI-205-51C1 | CDRL3 | SSYTKSGTLV |
| 53 | 52 | NI-205-21G2 | CDRH1 | SYTLH |
| | 53 | NI-205-21G2 | CDRH2 | WINAAFINTKYSQKFQG |
| | 54 | NI-205-21G2 | CDRH3 | RASGSNGLDV |
| | 55 | NI-205-21G2 | CDRL1 | QASRDITNYLN |
| | 56 | NI-205-21G2 | CDRL2 | DASYLET |
| | 57 | NI-205-21G2 | CDRL3 | QQYDSVPLT |
| 54 | 58 | NI-205-3F10 | CDRH1 | SQAMS |
| | 59 | NI-205-3F10 | CDRH2 | ALSRTGDYTWYADSVRG |
| | 60 | NI-205-3F10 | CDRH3 | NYYSSFGYNWAAFHI |
| | 61 | NI-205-3F10 | CDRL1 | RASQDVNNNYLA |
| | 62 | NI-205-3F10 | CDRL2 | GASRRAT |
| | 63 | NI-205-3F10 | CDRL3 | QQYGGSPPYT |
| 55 | 64 | NI-205-8A2 | CDRH1 | DHGMH |
| | 65 | NI-205-8A2 | CDRH2 | VIWLDGSSRFYADSVEG |
| | 66 | NI-205-8A2 | CDRH3 | DRVASEGTAFDV |
| | 67 | NI-205-8A2 | CDRL1 | WASQNVNHYLV |
| | 68 | NI-205-8A2 | CDRL2 | DTSVRAA |
| | 69 | NI-205-8A2 | CDRL3 | QHRSDWT |
| 56 | 70 | NI-205-113C4 | CDRH1 | NYYMH |
| | 71 | NI-205-113C4 | CDRH2 | IINPSGGRTSYAQKFQG |
| | 72 | NI-205-113C4 | CDRH3 | QRPSGYSGYGPSESYGNPTDDAFDV |
| | 73 | NI-205-113C4 | CDRL1 | GGNNIGSRGVH |
| | 74 | NI-205-113C4 | CDRL2 | DDSDRPS |
| | 75 | NI-205-113C4 | CDRL3 | QVWDNSSDHLVV |
| 57 | 76 | NI-205-87E7 | CDRH1 | SYAMS |
| | 77 | NI-205-87E7 | CDRH2 | AISGGGDRTYSADSVKG |
| | 78 | NI-205-87E7 | CDRH3 | GGGGEMTAVTMDGTYYGMDV |
| | 79 | NI-205-87E7 | CDRL1 | TGTSSNVGTYKFVS |
| | 80 | NI-205-87E7 | CDRL2 | DVTKRPS |
| | 81 | NI-205-87E7 | CDRL3 | CSYAGSYTYV |
| 58 | 82 | NI-205-21G1 | CDRH1 | SHGMH |
| | 83 | NI-205-21G1 | CDRH2 | VISYDASNKSYADSVKG |
| | 84 | NI-205-21G1 | CDRH3 | AFSSSASGGY |
| | 85 | NI-205-21G1 | CDRL1 | RSSQSLVHSDGVTYLN |
| | 86 | NI-205-21G1 | CDRL2 | KVSNRDS |
| | 87 | NI-205-21G1 | CDRL3 | MQGTHWPPWT |
| 59 | 88 | NI-205-68G5 | CDRH1 | SYGMH |
| | 89 | NI-205-68G5 | CDRH2 | IIYYDSSQRYYADSVKG |
| | 90 | NI-205-68G5 | CDRH3 | DLPFHYHRSASFAPSDT |
| | 91 | NI-205-68G5 | CDRL1 | RASQAVINNYLA |
| | 92 | NI-205-68G5 | CDRL2 | AASSRAT |
| | 93 | NI-205-68G5 | CDRL3 | QQYGTSPIT |
| 60 | 94 | NI-205-20A1 | CDRHI | SYRMN |
| | 95 | NI-205-20A1 | CDRH2 | YISTSSSTIYYADSVKG |
| | 96 | NI-205-20A1 | CDRH3 | AFDY |
| | 97 | NI-205-20A1 | CDRL1 | RASQSVSSSYLA |
| | 98 | NI-205-20A1 | CDRL2 | GASSRAT |
| | 99 | NI-205-20A1 | CDRL3 | QQYGSSPFT |
| 61 | 100 | NI-205-41D1 | CDRH1 | TYYMS |
| | 101 | NI-205-41D1 | CDRH2 | NIKQDGSEKYYVDSVKG |
| | 102 | NI-205-41D1 | CDRH3 | PPGW |
| | 103 | NI-205-41D1 | CDRL1 | KSSQSLLHSDGKTYLY |
| | 104 | NI-205-41D1 | CDRL2 | EVSNRFS |
| | 105 | NI-205-41D1 | CDRL3 | MQSIQLPVT |
| 62 | 106 | CASH-9C5 | CDRHI | DYYMY |
| | 107 | CASH-9C5 | CDRH2 | TISDGGSYTSYPDSVKG |
| | 108 | CASH-9C5 | CDRH3 | DYYGSSSYTSGFAY |
| | 109 | CASH-9C5 | CDRL1 | RSSQSIVHSNGNTYLE |
| | 110 | CASH-9C5 | CDRL2 | KVSNRFS |
| | 111 | CASH-9C5 | CDRL3 | FQGSHVPGT |
| 63 | 112 | CASH-2F7 | CDRH1 | SYYMS |
| | 113 | CASH-2F7 | CDRH2 | TINSNGGSTYYPDTVKG |
| | 114 | CASH-2F7 | CDRH3 | QNYEGAY |
| | 115 | CASH-2F7 | CDRL1 | RSSQSIVHSNGNTYLE |
| | 116 | CASH-2F7 | CDRL2 | KVSNRFS |
| | 117 | CASH-2F7 | CDRL3 | FQSSHVPWT |
| 64 | 118 | 401-A2C6 | CDRH1 | RFGMH |
| | 119 | 401-A2C6 | CDRH2 | YIRSGSDIIYYADSVKG |
| | 120 | 401-A2C6 | CDRH3 | SGTTVPFDY |
| | 121 | 401-A2C6 | CDRL1 | KSSQSLLNSGDQKNYLA |
| | 122 | 401-A2C6 | CDRL2 | FASTRAS |
| | 123 | 401-A2C6 | CDRL3 | QQHYSIPLT |
| 65 | 124 | Control-6H4 | CDRHI | SGYWS |
| | 125 | Control-6H4 | CDRH2 | YISYRGSTYYNPSLKS |
| | 126 | Control-6H4 | CDRH3 | FDSDDYAMEY |
| | 127 | Control-6H4 | CDRL1 | SASSSVSSSHLY |
| | 128 | Control-6H4 | CDRL2 | STSNLAS |
| | 129 | Control-6H4 | CDRL3 | HQWSSFPFT |
| 66 | 130 | Control- | CDRH1 | NYSMN |
| | | INT41 | | |
| | 131 | Control- | CDRH2 | SISSSSEYIYYADFVKG |
| | | INT41 | | |
| | 132 | Control- | CDRH3 | PGYRKA |
| | | INT41 | | |
| | 133 | Control- | CDRL1 | AGTSSDVGGYNYVS |
| | | INT41 | | |
| | 134 | Control- | CDRL2 | EDSKRPS |
| | | INT41 | | |
| | 135 | Control- | CDRL3 | SYCASKGHWL |
| | | INT41 | | |
| 93 | 136 | 631B2A2 | CDRH1 | EYSIH |
| | 137 | 631B2A2 | CDRH2 | GINPDNGGTRYNQKFKG |
| | 138 | 631B2A2 | CDRH3 | ES |
| | 139 | 631B2A2 | CDRL1 | KSSQSLLNSDGKTYLN |
| | 140 | 631B2A2 | CDRL2 | LVSKLDS |
| | 141 | 631B2A2 | CDRL3 | WQGTHFPHT |
| 94 | 142 | 633B12C8 | CDRH1 | EYSMH |
| | 143 | 633B12C8 | CDRH2 | GINPNNGGTSYNQKFKG |
| | 144 | 633B12C8 | CDRH3 | ES |
| | 145 | 633B12C8 | CDRL1 | KSSQSLLHSDGKTYLN |
| | 146 | 633B12C8 | CDRL2 | LVSKLDS |
| | 147 | 633B12C8 | CDRL3 | WQGTHFPHT |
| 95 | 148 | 634H10H7 | CDRH1 | DTYMH |
| | 149 | 634H10H7 | CDRH2 | RIDPANSNTKFDPKFQG |
| | 150 | 634H10H7 | CDRH3 | FYGGSHWYFDV |
| | 151 | 634H10H7 | CDRL1 | KASQDIKSYLS |
| | 152 | 634H10H7 | CDRL2 | YATSLAD |
| | 153 | 634H10H7 | CDRL3 | LQQGESPYT |
| 96 | 154 | 636E5B8 | CDRH1 | NYGMS |
| | 155 | 636E5B8 | CDRH2 | TISSGGKYINYLDSLKG |
| | 156 | 636E5B8 | CDRH3 | DYGSGWAWFAY |
| | 157 | 636E5B8 | CDRL1 | TLSSQHSTYTIE |
| | 158 | 636E5B8 | CDRL2 | LKKDGSHSTGD |
| | 159 | 636E5B8 | CDRL3 | GVGDTIKEQFVYV |
| 97 | 160 | 641H1E7 | CDRH1 | NYGVH |
| | 161 | 641H1E7 | CDRH2 | LMWAGGSTNYNSALMS |
| | 162 | 641H1E7 | CDRH3 | YRTGFAY |
| | 163 | 641H1E7 | CDRL1 | RSSQSIVHTIGNTYLE |
| | 164 | 641H1E7 | CDRL2 | KVSNRFS |
| | 165 | 641H1E7 | CDRL3 | FQGSHVPFT |
| 98 | 166 | 642A10B11 | CDRH1 | KYWMH |
| | 167 | 642A10B11 | CDRH2 | EINPSNGRTNYNEKFKS |
| | 168 | 642A10B11 | CDRH3 | YMDY |
| | 169 | 642A10B11 | CDRL1 | KSSQSLFDRDGKTYLN |
| | 170 | 642A10B11 | CDRL2 | LVSKLDS |
| | 171 | 642A10B11 | CDRL3 | WQGTHFPWT |
| 99 | 172 | 642D12B4 | CDRH1 | DPYMH |
| | 173 | 642D12B4 | CDRH2 | RIDPADGNTKYDPKFQG |
| | 174 | 642D12B4 | CDRH3 | FYGSSHWYFDV |
| | 175 | 642D12B4 | CDRL1 | KASQDIKRYLS |
| | 176 | 642D12B4 | CDRL2 | YATSLAD |
| | 177 | 642D12B4 | CDRL3 | LQQGESPYT |
| 100 | 178 | 646B7F7 | CDRH1 | NFGVH |
| | 179 | 646B7F7 | CDRH2 | IMWAGGSTNYNSALMS |
| | 180 | 646B7F7 | CDRH3 | YKTGFAY |
| | 181 | 646B7F7 | CDRL1 | RSSQSIVHAIGNTYLE |
| | 182 | 646B7F7 | CDRL2 | KVSNRFS |
| | 183 | 646B7F7 | CDRL3 | FQGSHVPFT |
| 101 | 184 | 712A6B10 | CDRH1 | EYTIH |
| | 185 | 712A6B10 | CDRH2 | WFHPENDNIKYNENFKD |
| | 186 | 712A6B10 | CDRH3 | TSGYGDY |
| | 187 | 712A6B10 | CDRL1 | KSSQSLLPSDGKTYLN |
| | 188 | 712A6B10 | CDRL2 | LVSKLDS |
| | 189 | 712A6B10 | CDRL3 | WQGTHFPPT |
| 102 | 190 | 809D9C2 | CDRH1 | DYSMH |
| | 191 | 809D9C2 | CDRH2 | VISTYYGDTTYNQKFKG |
| | 192 | 809D9C2 | CDRH3 | YGNFPASFSY |
| | 193 | 809D9C2 | CDRL1 | RSSKSLLHSNGNTYLY |
| | 194 | 809D9C2 | CDRL2 | RMSNLAS |
| | 195 | 809D9C2 | CDRL3 | MQHLEYPFT |
| 103 | 196 | 809F12D8 | CDRH1 | RNGVQ |
| | 197 | 809F12D8 | CDRH2 | VIWPGGSTNCNSALMS |
| | 198 | 809F12D8 | CDRH3 | VGGNYVWDYNNYA |
| | 199 | 809F12D8 | CDRL1 | RSSQNIVHSIGNTYLE |
| | 200 | 809F12D8 | CDRL2 | KVSNRFS |
| | 201 | 809F12D8 | CDRL3 | FQGSHVPYT |

TABLE 4
Amino acid sequences of the VHs of exemplary TDP-43 scFvs

| Construct | SEQ ID | | |
|---|---|---|---|
| ID | NO: | TDP-43 scFv | VH amino acid sequence |
| 50 | 202 | E6-VH7VK9 | QVQLQQSGGGLVQPGGSMKLSCVASGFTSSNYWLNWVRQSPERGLE |
| | | | WVAEIRLKSNNYATNYAESVKGRFTISRDDSKSSVYLQVNNLRAEDTG |
| | | | IYYCTRSTARATPYYFDYWGQGTTVTVSS |
| 51 | 203 | 3B12A | EVQLQQSGAELVKPGASVKLSCTASGFNIKDYYMHWVKQRTEQGLE |
| | | | WIGRIDPEDGETKYAPKFQGKATITADTSSNTAYLQLSSLTSEDTAVYY |
| | | | CTIIYYYGSRYVDYWGQGTTLTVSS |
| 52 | 204 | NI-205-51C1 | EVQLVESGGGLVQPGGSLRISCTTSGFIFSDYWMHWVRQAPGKGLTW |
| | | | VSRINLDGSDTIYADSVKGRFTISRDNDKNTLYLQMNSLRVEDTAIYYC |
| | | | ARSRKSVWGQGTMVTVSS |
| 53 | 205 | NI-205-21G2 | QVQLVQSGAEVKKPGASVKVSCKTSGYSFTSYTLHWVRQAPGHRPE |
| | | | WMGWINAAFINTKYSQKFQGRITLTRDTSANIAYLELRSLTTEDTAVY |
| | | | YCARRASGSNGLDVWGQGTTVTVSS |
| 54 | 206 | NI-205-3F10 | EVQLLESGGDLVQPGGSLRLSCAASGFTFSSQAMSWVRQAPGKGLEW |
| | | | VSALSRTGDYTWYADSVRGRFTVSRDDSKNIFYLEMNSLRAEDTAVY |
| | | | YCAKNYYSSFGYNWAAFHIWGQGTMVTVSS |
| 55 | 207 | NI-205-8A2 | QVQLVESGGGVVQPGKSLRLSCAASGFTFRDHGMHWVRQAPGKGLE |
| | | | WVAVIWLDGSSRFYADSVEGRFTISRDNSKNTLYLQLTSLRAEDTAIY |
| | | | YCARDRVASEGTAFDVWGQGTMVTVSS |
| 56 | 208 | NI-205- | QVQLVQSGAEVKKPGASVKVSCKASGYTFTNYYMHWVRQAPGQGLE |
| | | 113C4 | WMGIINPSGGRTSYAQKFQGRASMTRDTSTSTVYMEVISLRSEDTAVY |
| | | | YCARQRPSGYSGYGPSESYGNPTDDAFDVWGQGTTVTVSS |
| 57 | 209 | NI-205-87E7 | EVQLLESGGGLVQPGGSLRLSCAASGFTFSSYAMSWVRQAPGKGLEW |
| | | | VSAISGGGDRTYSADSVKGRFTISRDNSKNTLYLQINSLRVEDTAVYYC |
| | | | AQGGGGEMTAVTMDGTYYGMDVWGQGTTVTVSS |
| 58 | 210 | NI-205-21G1 | QVQLVESGGGVVQPGMSLRLSCAASGFSFSSHGMHWVRQTPGKGLE |
| | | | WLAVISYDASNKSYADSVKGRFTISRDNSKKTLYLQMDSLRVEDTAL |
| | | | YYCANAFSSSASGGYWGQGTLVTVSS |
| 59 | 211 | NI-205-68G5 | QVQLVESGGGVVQPGRSLRLSCAASGFTFSSYGMHWVRQAPGKGLE |
| | | | WVAIIYYDSSQRYYADSVKGRFTISRDNSKNALYLQMNSLRAEDTALY |
| | | | YCARDLPFHYHRSASFAPSDTWGQGTLVTVSS |
| 60 | 212 | 60-NI-205- | EVQLVESGGGLVQPGGSLRLSCAASGFTFSSYRMNWVRQAPGKGLEW |
| | | 20A1 | VSYISTSSSTIYYADSVKGRFTISRDNAKNSLYLQMNSLRAEDTAVYYC |
| | | | ARAFDYWGQGTLVTVSS |
| 61 | 213 | NI-205-41D1 | EVQLVESGGGLVQPGGSLRLSCAASGFTFSTYYMSWVRQAPGKGLEW |
| | | | VANIKQDGSEKYYVDSVKGRFTISRDNARNSLYLQMHSLRAEDTAVY |
| | | | YCASPPGWWGQGTLVTVSS |
| 62 | 214 | CASH-9C5 | EVQLVESGGGLVKPGGSLKLSCAASGFTFSDYYMYWVRQTPEKRLEW |
| | | | VATISDGGSYTSYPDSVKGRFTISRDNAKNNLYLQMSSLRSEDTAMYY |
| | | | CARDYYGSSSYTSGFAYWGQGTLVTVSS |
| 63 | 215 | CASH-2F7 | DVKLVESGGGLVKLGGSLKLSCAASGFTFSSYYMSWVRQTPEKRLEL |
| | | | VATINSNGGSTYYPDTVKGRITISRDNAKNTLQLQMSSLRSEDTALYY |
| | | | CVRQNYEGAYWGQGTLVTVSS |
| 64 | 216 | 401-A2C6 | DVQLVESGGGLVQPGGSRRLSCAASGFTFSRFGMHWVRQAPEKGLEW |
| | | | VAYIRSGSDIIYYADSVKGRFTISRDNPENTLFLQMTSLRSEDTAMYYC |
| | | | ARSGTTVPFDYWGQGTSLTVSS |
| 65 | 217 | Control-6H4 | EVQLQESGPSLVKPSQTLSLTCSVTGDSVTSGYWSWIRQFPGNKLDYM |
| | | | GYISYRGSTYYNPSLKSRISITRDTSKNQVYLQLKSVSSEDTATYYCSYF |
| | | | DSDDYAMEYWGQGTSVTVSS |
| 66 | 218 | Control- | EVQLVVSGGGLVKPGGSMILSCAASGFTFSNYSMNWVRQAPGKGLE |
| | | INT41 | WVSSISSSSEYIYYADFVKGRFTISRDNAKNSLYLQMDSLRAEDTAVY |
| | | | YCAWPGYRKAWGRGTLVTVSS |
| 93 | 219 | 631B2A2 | EVQLQQSGPELVKPGASVKISCKTSGYTFTEYSIHWVKQSHGESLEWIG |
| | | | GINPDNGGTRYNQKFKGKATLTVDKSSSTAYMDLRSLTSEDSAVYYC |
| | | | ARESWGQGTTLTVSS |
| 94 | 220 | 633B12C8 | EVQLQQSGPELVKPGASVKISCKTSGFTFTEYSMHWVKQSHGKSLEWI |
| | | | GGINPNNGGTSYNQKFKGKATLTVDKSSSTAYMELRSLTSEDSAVYYC |
| | | | ARESWGQGTTLTVSS |
| 95 | 221 | 634H10H7 | EVQLQQSGAELVKPGASVRLSCTASGFNIKDTYMHWVKQRPEQGLEW |
| | | | IGRIDPANSNTKFDPKFQGKATITSDTSSNTAYLQLSSLTSEDTAVYYC |
| | | | ARFYGGSHWYFDVWGAGTTVTVSS |
| 96 | 222 | 636E5B8 | EVHLVESGGDLVMPGGSLKLSCAASGFTFSNYGMSWVRQTPDKRLE |
| | | | WVATISSGGKYINYLDSLKGRFTISRDNAKNTLYLQMSSLKSEDTAMY |
| | | | YCAKDYGSGWAWFAYWGQGTLVTVSA |
| 97 | 223 | 641H1E7 | QVQLKESGPGLVAPSQSLSITCTVSGFSLTNYGVHWVRQPPGKGLEWL |
| | | | GLMWAGGSTNYNSALMSRLSISKDNSKSQVFLKMNSLQTDDTAMYY |
| | | | CVIYRTGFAYWGQGTLVTVSA |
| 98 | 224 | 642A10B11 | QVQLQQPGAELVKPGASVKLSCKASGYTFTKYWMHWVKQRPGQGLE |
| | | | WIGEINPSNGRTNYNEKFKSKATLTVDKSSSTAYMQLSSLTSEDSAVY |
| | | | YCARYMDYWGQGTSVTVSS |
| 99 | 225 | 642D12B4 | EVQLQQSGAELVKPGASVRLSCTASGFNIKDPYMHWVRQRPKQGLEW |
| | | | IGRIDPADGNTKYDPKFQGKATLTADTSSNVAYLHLSSLTSEDTAVYY |
| | | | CARFYGSSHWYFDVWGAGTTVTVSS |
| 100 | 226 | 646B7F7 | QVQLKESGPGLVAPSQSLSITCTVSGFSLTNFGVHWVRQPPGKGLEWL |
| | | | GIMWAGGSTNYNSALMSRLSISKDNSKSQVFLKMNSLQTDDTAMYYC |
| | | | VIYKTGFAYWGQGTLVTVSA |
| 101 | 227 | 712A6B10 | QVQLQQSGAELVKPGTSVKLSCKASAYTFTEYTIHWIKQKSGQGLEWI |
| | | | GWFHPENDNIKYNENFKDKATLTADRSSSTVYMELSRLTSEDSAVYFC |
| | | | AGTSGYGDYWGQGTTLTVSS |
| 102 | 228 | 809D9C2 | QVQLQQSGAELVRPGVSVKISCKGSGYKFTDYSMHWVKQSHTKSLE |
| | | | WIGVISTYYGDTTYNQKFKGKATITVDKSSSTAYMELARLTSEDSAIY |
| | | | YCATYGNFPASFSYWGQGTLVTVSA |
| 103 | 229 | 809F12D8 | QVQLKESGPGLVAPSQSLSITCTVSGFSLNRNGVQWVRQPPGKGLEWL |
| | | | GVIWPGGSTNCNSALMSRLSISKDNSKSQVFLKMNSLHTDDTGIYYCA |
| | | | RVGGNYVWDYNNYAWGQGTLVTVSA |

TABLE 5
Amino acid sequences of the VLs of exemplary TDP-43 scFvs

| Construct | SEQ ID | | |
|---|---|---|---|
| ID | NO: | TDP-43 scFv | VL amino acid sequence |
| 50 | 230 | E6-VH7VK9 | DIELTQSPSSLAVSAGEKVTMSCKSSQSLLNSRARKNFLTWYQQKPGQ |
| | | | SPKLLIYWASTRESGVPDRFTGSGSGTDFTLTISSVQAEDLAVYYCKQS |
| | | | YNLYTFGGGTKLEIK |
| 51 | 231 | 3B12A | EIVLTQSPTTMAASPGEKITITCSASSSISSSYLHWYQQKPGFSPKLLIYR |
| | | | TSNLASGVPARFSGSGSGTSYSLTIGTMEAEDVATYYCQQGSSIPLTFG |
| | | | SGTKLEIK |
| 52 | 232 | NI-205-51C1 | QSALTQPASVSGSPGQSITISCTGSNTDVGAYDYVSWSQQLPGKAPKFV |
| | | | IFDVDVRPSGISDRFSGSKSGNTASLTISGLQAEDEADYYCSSYTKSGTL |
| | | | VFGGGTKVTVV |
| 53 | 233 | NI-205-21G2 | DIQMTQSPSSLSASVGDRITITCQASRDITNYLNWYQQKPGKAPKLLIY |
| | | | DASYLETGVPSTFSGSGSGTHFTLTISSLQPDDFATYYCQQYDSVPLTF |
| | | | GGGTKVEIK |
| 54 | 234 | NI-205-3F10 | EIVLTQSPGTLSLSPGERATLSCRASQDVNNNYLAWYQQKPGQAPRLLI |
| | | | YGASRRATGVPDRFSGRGSGTDFTLTINRLEPEDFAMYFCQQYGGSPP |
| | | | YTFGQGTKLEIK |
| 55 | 235 | NI-205-8A2 | EIVLTQSPATLSLSPGERATLSCWASQNVNHYLVWYQQRPGQAPRLLL |
| | | | YDTSVRAAGIPARFIGSGSGTHFTLTISSLEPEDSAVYYCQHRSDWTFG |
| | | | QGTKVEIK |
| 56 | 236 | NI-205- | SYVLTQPPSVSVAPGQTARITCGGNNIGSRGVHWYQQRPGQAPVLVV |
| | | 113C4 | YDDSDRPSGIPERFSGSNSGDTATLTISRVEVGDEADYYCQVWDNSSD |
| | | | HLVVFGGGTKLTVL |
| 57 | 237 | NI-205-87E7 | QSALTQPRSVSGSPGQSITISCTGTSSNVGTYKFVSWYQQHPGKAPKLM |
| | | | IYDVTKRPSGVPDRFSGSKSGNTASLTISGLQAEDEADYYCCSYAGSYT |
| | | | YVFGSGTKVTVL |
| 58 | 238 | NI-205-21G1 | DVVMTQSPLSLPVTLGQPASISCRSSQSLVHSDGVTYLNWFQQRPGQS |
| | | | PRRLIYKVSNRDSGVPDRFSGSGSGTDFTLEISRVEAEDVGIYYCMQGT |
| | | | HWPPWTFGQGTKVEIK |
| 59 | 239 | NI-205-68G5 | EIVLTQSPGTLSLSPGERATLSCRASQAVINNYLAWYQQKPGQAPRLL |
| | | | VYAASSRATGIPDRFYGSGSGADFTLTISRLEPEDFAVYYCQQYGTSPIT |
| | | | FGQGTRLEIK |
| 60 | 240 | 60-NI-205- | EIVLTQSPGTLSLSPGERATLSCRASQSVSSSYLAWYQQKPGQAPRLLI |
| | | 20A1 | YGASSRATGIPDRFSGSGSGTDFTLTIIRLEPEDFAVYYCQQYGSSPFTF |
| | | | GQGTKVEIK |
| 61 | 241 | NI-205-41D1 | DIVMTQTPLSLSVTPGQPASISCKSSQSLLHSDGKTYLYWYLQKPGQPP |
| | | | QLLIYEVSNRFSGVPDRFSGSGSGTDFTLKISRVEAEDVGVYYCMQSIQ |
| | | | LPVTFGGGTKVEIK |
| 62 | 242 | CASH-9C5 | DVLMTQTPLSLPVSLGDQASISCRSSQSIVHSNGNTYLEWYLQKPGQSP |
| | | | KLLIYKVSNRFSGVPDRFSGSGSGTDFTLKISRVEAEDLGVYYCFQGSH |
| | | | VPGTFGGGTKLEIK |
| 63 | 243 | CASH-2F7 | DVLMTQTPLSLPVTLGDQASISCRSSQSIVHSNGNTYLEWYLQKPGQSP |
| | | | KLLIYKVSNRFSGVPDRFSGSGSGTDFTLKISRVEAEDLGVYYCFQSSH |
| | | | VPWTFGGGTKLEIK |
| 64 | 244 | 401-A2C6 | DIVMTQSPSSLAMSVGQKVTMSCKSSQSLLNSGDQKNYLAWYQQKPG |
| | | | QSPELLLYFASTRASGVPDRFIGSGSGTDFSLTISSVQAEDLADYFCQQH |
| | | | YSIPLTFGAGTKLELK |
| 65 | 245 | Control-6H4 | QIVLTQSPAIMSASPGEKVTLTCSASSSVSSSHLYWYQQKPGSSPKLWI |
| | | | YSTSNLASGVPARFSGSGSGTSYSLTISSMEAEDAASYFCHQWSSFPFTF |
| | | | GSGTKLEIK |
| 66 | 246 | Control- | QSVLTQPASVSGSPGQSITISCAGTSSDVGGYNYVSWYQQHPGKAPKL |
| | | INT41 | MIYEDSKRPSGVSNRFSGSKSGNTASLTISGLRAEDEADYYCSYCASKG |
| | | | HWLFGGGTKLAVL |
| 93 | 247 | 631B2A2 | DVVMTQTPLTLSVTIGQPASISCKSSQSLLNSDGKTYLNWLLQRPGQSP |
| | | | KRLIYLVSKLDSRIPDRFTGSGSGTDFTLKISRVEAEDLGVYYCWQGTH |
| | | | FPHTFGSGTKLELK |
| 94 | 248 | 633B12C8 | DVVMTQTPLTLSVTIGQPASISCKSSQSLLHSDGKTYLNWLLQRPGQSP |
| | | | KRLIYLVSKLDSRIPDRFTGSGSGTDFTLKISRVEAEDLGVYYCWQGTH |
| | | | FPHTFGAGTKLELK |
| 95 | 249 | 634H10H7 | DIKMTQSPSSMYASLGERVTITCKASQDIKSYLSWYQHKPWKSPKALI |
| | | | YYATSLADGVPSRFSGSGSGQDYSLTISSLESDDTATYYCLQQGESPYT |
| | | | FGGGTKLEIK |
| 96 | 250 | 636E5B8 | QLVLTQSSSASFSLGASAKLTCTLSSQHSTYTIEWYQQQPLKPPKYVME |
| | | | LKKDGSHSTGDGIPDRFSGSSSGADRYLSISNIQPEDEAIYICGVGDTIKE |
| | | | QFVYVFGGGTKVTVL |
| 97 | 251 | 641H1E7 | DVLMTQTPLSLPVSLGDQASISCRSSQSIVHTIGNTYLEWYLQKPGQSP |
| | | | KLLIYKVSNRFSGVPDRFSGSGSGTDFTLKISRVEAEDLGVYYCFQGSH |
| | | | VPFTFGSGTKLEIK |
| 98 | 252 | 642A10B11 | DVVMTQTPLTLSVTIGQPASISCKSSQSLFDRDGKTYLNWLLQRPGQSP |
| | | | KRLIYLVSKLDSGVPDRFTGSGSGTDFTLKISRVEAEDLGVYYCWQGT |
| | | | HFPWTFGGGTKLEIK |
| 99 | 253 | 642D12B4 | DIKMTQSPSSMYASLGERVTITCKASQDIKRYLSWYQQKPWKSPKILIY |
| | | | YATSLADGVPSRFSGTGSGQDYSLTISSLESDDVATYYCLQQGESPYTF |
| | | | GGGTKLEIK |
| 100 | 254 | 646B7F7 | DVLMTQTPLSLPVSLGDQASISCRSSQSIVHAIGNTYLEWYLQKPGQSP |
| | | | KLLIYKVSNRFSGVPDRFSGSGSGTDFTLKISRVEAEDLGVYYCFQGSH |
| | | | VPFTFGSGTKLEIK |
| 101 | 255 | 712A6B10 | DVVMTQIPLTLSITIGQPASISCKSSQSLLPSDGKTYLNWLLQRPGQSPK |
| | | | RLIYLVSKLDSGVPDRFTGSGSGTDFTLKISRVEADDLGVYYCWQGTH |
| | | | FPPTFGAGTKLELK |
| 102 | 256 | 809D9C2 | DIVMTQAAPSIPVTPGESVSISCRSSKSLLHSNGNTYLYWFLQRPGQSPQ |
| | | | LLIYRMSNLASGVPDRFSGSGSGTAFTLRISRVEAEDVGVYYCMQHLE |
| | | | YPFTFGSGTKLEIK |
| 103 | 257 | 809F12D8 | DVLMTQTPLSLPVSLGDQASISCRSSQNIVHSIGNTYLEWYLQKPGQSP |
| | | | KLLIYKVSNRFSGVPDRFSGSGSGTDFTLKISRVEAEDLGVYYCFQGSH |
| | | | VPYTFGGGTKLEIR |

In an embodiment, the scFv that specifically binds TDP-43 comprises a heavy chain variable domain comprising the CDRH1, CDRH2, CDRH3, amino acid sequences of SEQ ID NOs: 34, 35, and 36; 40, 41, and 42; 46, 47, and 48; 52, 53, and 54; 58, 59, and 60; 64, 65, and 66; 70, 71, and 72; 76, 77, and 78; 82, 83, and 84; 88, 89, and 90; 94, 95, and 96; 100, 101, and 102; 106, 107, and 108; 112, 113, and 114; 118, 119, and 120; 124, 125, and 126; 130, 131, and 132; 136, 137, and 138; 142, 143, and 144; 148, 149, and 150; 154, 155, and 156; 160, 161, and 162; 166, 167, and 168; 172, 173, and 174; 178, 179, and 180; 184, 185, and 186; 190, 191, and 192; or 196, 197, and 198, respectively.

In an embodiment, the scFv that specifically binds TDP-43 comprises a light chain variable domain comprising the CDRL1, CDRL2, and CDRL3 amino acid sequences of SEQ ID NOs: 37, 38, and 39; 43, 44, and 45; 49, 50, and 51; 55, 56, and 57; 61, 62, and 63; 67, 68, and 69; 73, 74, and 75; 79, 80, and 81; 85, 86, and 87; 91, 92, and 93; 97, 98, and 99; 103, 104, and 105; 109, 110, and 111; 115, 116, and 117; 121, 122, and 123; 127, 128, and 129; 133, 134, and 135; 139, 140, and 141; 145, 146, and 147; 151, 152, and 153; 157, 158, and 159; 163, 164, and 165; 169, 170, and 171; 175, 176, and 177; 181, 182, and 183; 187, 188, and 189; 193, 194, and 195; or 199, 200, and 201, respectively.

In an embodiment, the scFv that specifically binds TDP-43 comprises a heavy chain variable domain and a light chain variable domain comprising the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the heavy chain variable domain comprises an amino acid sequence selected from the group consisting of SEQ ID NOs: 202-229; and the light chain variable domain comprises an amino acid sequence selected from the group consisting of SEQ ID NOs: 230-257.

In an embodiment, the heavy chain variable domain and light chain variable domain comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising an amino acid sequence selected from the group consisting of SEQ ID NOs: 1-19.

In an embodiment, the chimeric molecule comprises an scFv that specifically binds to human TDP-43 and a degron comprising the amino acid sequence of SEQ ID NO: 1, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 2, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 3, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 4, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 5, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 6, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 7, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 8, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 9, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 10, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 11, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 12, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 13, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 14, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 15, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 16, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 17, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 18, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 19, wherein the scFv comprises the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

In an embodiment, the chimeric molecule comprises an scFv that specifically binds to human TDP-43 and a degron comprising the amino acid sequence of SEQ ID NO: 1, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 2, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 3, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 4, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 5, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 6, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 7, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 8, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 9, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 10, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 11, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 12, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 13, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 14, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 15, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 16, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 17, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 18, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the chimeric molecule comprises an scFv that specifically binds to human TDP-43 and a degron comprising the amino acid sequence of SEQ ID NO: 19, wherein the heavy chain variable domain and light chain variable domain of the scFv comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

In an embodiment, the polypeptide is an scFv or nanobody that specifically binds to human huntingtin. In an embodiment, the scFv or nanobody specifically binds to human huntingtin at an epitope comprising amino acids 1-17, 18-38, 39-49, 50-64, 62-65, 67-76, 73-87, 80-89, 575-584, or 585-516 of SEQ ID NO: 291.

Amino acid sequence of huntingtin
(SEQ ID NO: 291)
MATLEKLMKAFESLKSFQQQQQQQQQQQQQQQQQQQQQPPPPPPPPPPPQLPQPPPQA
QPLLPQPQPPPPPPPPPPGPAVAEEPLHRPKKELSATKKDRVNHCLTICENIVAQSVRNSP
EFQKLLGIAMELFLLCSDDAESDVRMVADECLNKVIKALMDSNLPRLQLELYKEIKKNG
APRSLRAALWRFAELAHLVRPQKCRPYLVNLLPCLTRTSKRPEESVQETLAAAVPKIMA
SFGNFANDNEIKVLLKAFIANLKSSSPTIRRTAAGSAVSICQHSRRTQYFYSWLLNVLLGL
LVPVEDEHSTLLILGVLLTLRYLVPLLQQQVKDTSLKGSFGVTRKEMEVSPSAEQLVQV
YELTLHHTQHQDHNVVTGALELLQQLFRTPPPELLQTLTAVGGIGQLTAAKEESGGRSR
SGSIVELIAGGGSSCSPVLSRKQKGKVLLGEEEALEDDSESRSDVSSSALTASVKDEISGE
LAASSGVSTPGSAGHDIITEQPRSQHTLQADSVDLASCDLTSSATDGDEEDILSHSSSQVS
AVPSDPAMDLNDGTQASSPISDSSQTTTEGPDSAVTPSDSSEIVLDGTDNQYLGLQIGQP
QDEDEEATGILPDEASEAFRNSSMALQQAHLLKNMSHCRQPSDSSVDKFVLRDEATEPG
DQENKPCRIKGDIGQSTDDDSAPLVHCVRLLSASFLLTGGKNVLVPDRDVRVSVKALAL
SCVGAAVALHPESFFSKLYKVPLDTTEYPEEQYVSDILNYIDHGDPQVRGATAILCGTLI
CSILSRSRFHVGDWMGTIRTLTGNTFSLADCIPLLRKTLKDESSVTCKLACTAVRNCVMS
LCSSSYSELGLQLIIDVLTLRNSSYWLVRTELLETLAEIDFRLVSFLEAKAENLHRGAHHY
TGLLKLQERVLNNVVIHLLGDEDPRVRHVAAASLIRLVPKLFYKCDQGQADPVVAVAR
DQSSVYLKLLMHETQPPSHFSVSTITRIYRGYNLLPSITDVTMENNLSRVIAAVSHELITST
TRALTFGCCEALCLLSTAFPVCIWSLGWHCGVPPLSASDESRKSCTVGMATMILTLLSSA
WFPLDLSAHQDALILAGNLLAASAPKSLRSSWASEEEANPAATKQEEVWPALGDRALV
PMVEQLFSHLLKVINICAHVLDDVAPGPAIKAALPSLTNPPSLSPIRRKGKEKEPGEQASV
PLSPKKGSEASAASRQSDTSGPVTTSKSSSLGSFYHLPSYLKLHDVLKATHANYKVTLDL
QNSTEKFGGFLRSALDVLSQILELATLQDIGKCVEEILGYLKSCFSREPMMATVCVQQLL
KTLFGTNLASQFDGLSSNPSKSQGRAQRLGSSSVRPGLYHYCFMAPYTHFTQALADASL
RNMVQAEQENDTSGWFDVLQKVSTQLKTNLTSVTKNRADKNAIHNHIRLFEPLVIKAL
KQYTTTTCVQLQKQVLDLLAQLVQLRVNYCLLDSDQVFIGFVLKQFEYIEVGQFRESEA
IIPNIFFFLVLLSYERYHSKQIIGIPKIIQLCDGIMASGRKAVTHAIPALQPIVHDLFVLRGTN
KADAGKELETQKEVVVSMLLRLIQYHQVLEMFILVLQQCHKENEDKWKRLSRQIADIIL
PMLAKQQMHIDSHEALGVLNTLFEILAPSSLRPVDMLLRSMFVTPNTMASVSTVQLWIS
GILAILRVLISQSTEDIVLSRIQELSFSPYLISCTVINRLRDGDSTSTLEEHSEGKQIKNLPEE
TFSRFLLQLVGILLEDIVTKQLKVEMSEQQHTFYCQELGTLLMCLIHIFKSGMFRRITAAA
TRLFRSDGCGGSFYTLDSLNLRARSMITTHPALVLLWCQILLLVNHTDYRWWAEVQQT
PKRHSLSSTKLLSPQMSGEEEDSDLAAKLGMCNREIVRRGALILFCDYVCQNLHDSEHL
TWLIVNHIQDLISLSHEPPVQDFISAVHRNSAASGLFIQAIQSRCENLSTPTMLKKTLQCLE
GIHLSQSGAVLTLYVDRLLCTPFRVLARMVDILACRRVEMLLAANLQSSMAQLPMEEL
NRIQEYLQSSGLAQRHQRLYSLLDRFRLSTMQDSLSPSPPVSSHPLDGDGHVSLETVSPD
KDWYVHLVKSQCWTRSDSALLEGAELVNRIPAEDMNAFMMNSEFNLSLLAPCLSLGM
SEISGGQKSALFEAAREVTLARVSGTVQQLPAVHHVFQPELPAEPAAYWSKLNDLFGDA
ALYQSLPTLARALAQYLVVVSKLPSHLHLPPEKEKDIVKFVVATLEALSWHLIHEQIPLS
LDLQAGLDCCCLALQLPGLWSVVSSTEFVTHACSLIYCVHFILEAVAVQPGEQLLSPERR
TNTPKAISEEEEEVDPNTQNPKYITAACEMVAEMVESLQSVLALGHKRNSGVPAFLTPLL
RNIIISLARLPLVNSYTRVPPLVWKLGWSPKPGGDFGTAFPEIPVEFLQEKEVFKEFIYRIN
TLGWTSRTQFEETWATLLGVLVTQPLVMEQEESPPEEDTERTQINVLAVQAITSLVLSA
MTVPVAGNPAVSCLEQQPRNKPLKALDTRFGRKLSIIRGIVEQEIQAMVSKRENIATHHL
YQAWDPVPSLSPATTGALISHEKLLLQINPERELGSMSYKLGQVSIHSVWLGNSITPLREE
EWDEEEEEEADAPAPSSPPTSPVNSRKHRAGVDIHSCSQFLLELYSRWILPSSSARRTPAI
LISEVVRSLLVVSDLFTERNQFELMYVTLTELRRVHPSEDEILAQYLVPATCKAAAVLG
MDKAVAEPVSRLLESTLRSSHLPSRVGALHGVLYVLECDLLDDTAKQLIPVISDYLLSNL
KGIAHCVNIHSQQHVLVMCATAFYLIENYPLDVGPEFSASIIQMCGVMLSGSEESTPSIIY
HCALRGLERLLLSEQLSRLDAESLVKLSVDRVNVHSPHRAMAALGLMLTCMYTGKEK
VSPGRTSDPNPAAPDSESVIVAMERVSVLFDRIRKGFPCEARVVARILPQFLDDFFPPQDI
MNKVIGEFLSNQQPYPQFMATVVYKVFQTLHSTGQSSMVRDWVMLSLSNFTQRAPVA
MATWSLSCFFVSASTSPWVAAILPHVISRMGKLEQVDVNLFCLVATDFYRHQIEEELDR
RAFQSVLEVVAAPGSPYHRLLTCLRNVHKVTTC

Amino acid sequences of exemplary huntingtin scFvs and nanobodies are listed in Tables 6-8 below.

TABLE 6
Amino acid sequences of the CDRs of
exemplary huntingtin scFvs and nanobodies

| Con- | SEQ | Huntingtin | | |
|---|---|---|---|---|
| struct | ID | scFv or | | Amino acid |
| ID | NO: | nanobody | CDR | sequence |
| 240 | 292 | C4 | CDRH1 | SYSMS |
| | 293 | C4 | CDRH2 | VISYDGSNKYYADSVKG |
| | 294 | C4 | CDRH3 | DRYFDL |
| | 295 | C4 | CDRL1 | TGTSSDIGAYNYVS |
| | 296 | C4 | CDRL2 | DVSNRPS |
| | 297 | C4 | CDRL3 | SSFANSGPL |
| 241 | 298 | VL12.3 | CDRL1 | SGSNSNIGSNTVN |
| | 299 | VL12.3 | CDRL2 | DDDLLAP |
| | 300 | VL12.3 | CDRL3 | ATWDDSLNGWV |
| 242 | 301 | NI-302.15E8 | CDRH1 | SYSMN |
| | 302 | NI-302.15E8 | CDRH2 | YTSSSRSNTKKYADSVKG |
| | 303 | NI-302.15E8 | CDRH3 | AGDFGELLTGEGYYGMDV |
| | 304 | NI-302.15E8 | CDRL1 | SGDELGDKYVG |
| | 305 | NI-302.15E8 | CDRL2 | QDAKRPS |
| | 306 | NI-302.15E8 | CDRL3 | QAWDSGTMV |
| 243 | 307 | MW1 | CDRH1 | DYYMY |
| | 308 | MW1 | CDRH2 | FISNGGGSTYYPDTVKG |
| | 309 | MW1 | CDRH3 | GRGYVWFAY |
| | 310 | MW1 | CDRL1 | TLSSQHSTYTIE |
| | 311 | MW1 | CDRL2 | LKKDGSHSTGD |
| | 312 | MW1 | CDRL3 | GVGDTIKEQFVYV |
| 244 | 313 | MW2 | CDRH1 | NYGMN |
| | 314 | MW2 | CDRH2 | WINTYTGEPTYADDSKG |
| | 315 | MW2 | CDRH3 | RGLLFAY |
| | 316 | MW2 | CDRL1 | TLSSQHSTYTIE |
| | 317 | MW2 | CDRL2 | LKKDGSHSTGD |
| | 318 | MW2 | CDRL3 | GVGDTIKEQFVYV |
| 245 | 319 | 1C2 | CDRH1 | TYGMN |
| | 320 | 1C2 | CDRH2 | WINTYSGVPTYADDFKG |
| | 321 | 1C2 | CDRH3 | RRSDGYSNYFDY |
| | 322 | 1C2 | CDRL1 | TLSRQHSTYTIE |
| | 323 | 1C2 | CDRL2 | LKKDGSHSTGD |
| | 324 | 1C2 | CDRL3 | GVGDTIKEQFVYV |
| 246 | 325 | 3B5H10 | CDRH1 | TYGMS |
| | 326 | 3B5H10 | CDRH2 | WINTYSGVPTYVDDFKG |
| | 327 | 3B5H10 | CDRH3 | GGDNYLWFAY |
| | 328 | 3B5H10 | CDRL1 | TLNSQHSTYTIE |
| | 329 | 3B5H10 | CDRL2 | LKKDGSHSTGD |
| | 330 | 3B5H10 | CDRL3 | GVGDTIKEQFVYV |
| 247 | 331 | MW7 | CDRH1 | DAWMD |
| | 332 | MW7 | CDRH2 | EIRSKANNHATYYAESVK |
| | | | | G |
| | 333 | MW7 | CDRH3 | AGFAY |
| | 334 | MW7 | CDRL1 | KSSQSLLNSSNQKNYLA |
| | 335 | MW7 | CDRL2 | FASTRES |
| | 336 | MW7 | CDRL3 | QQHYSTPWT |
| 248 | 337 | VHH2 | CDRH1 | YYAIG |
| | 338 | VHH2 | CDRH2 | CISATDGSTYYADSVKG |
| | 339 | VHH2 | CDRH3 | VRAPYSDYCNGYYDY |
| 249 | 340 | VHH4 | CDRH1 | YYAIG |
| | 341 | VHH4 | CDRH2 | CISSSDGSTYYADSVKG |
| | 342 | VHH4 | CDRH3 | VRAPYSDYCNGYYDY |
| 250 | 343 | NI-302.33C11 | CDRH1 | DFGMH |
| | 344 | NI-302.33C11 | CDRH2 | LIWYDGGYKYYADSVKG |
| | 345 | NI-302.33C11 | CDRH3 | HLEYCSRTTCYLGH |
| | 346 | NI-302.33C11 | CDRL1 | RASQGISDYLA |
| | 347 | NI-302.33C11 | CDRL2 | AASTLQT |
| | 348 | NI-302.33C11 | CDRL3 | QQLKTYPYT |
| 251 | 349 | Happ1 | CDRL1 | SGSSSNIGSNYVY |
| | 350 | Happ1 | CDRL2 | RNNQRPS |
| | 351 | Happ1 | CDRL3 | AAWDDSLCVALV |
| 252 | 352 | INT41 | CDRH1 | NYSMN |
| | 353 | INT41 | CDRH2 | SISSSSEYIYYADFVKG |
| | 354 | INT41 | CDRH3 | PGYRKA |
| | 355 | INT41 | CDRL1 | AGTSSDVGGYNYVS |
| | 356 | INT41 | CDRL2 | EDSKRPS |
| | 357 | INT41 | CDRL3 | SYCASKGHWL |
| 253 | 358 | NI302.63F3 | CDRH1 | TRSMN |
| | 359 | NI302.63F3 | CDRH2 | WINTNTGNRTYVQAFRG |
| | 360 | NI302.63F3 | CDRH3 | GAGGGYWFDS |
| | 361 | NI302.63F3 | CDRL1 | KSNQSLFYSSNNNNYLA |
| | 362 | NI302.63F3 | CDRL2 | WGSTRES |
| | 363 | NI302.63F3 | CDRL3 | HQYYHNPYT |
| 254 | 364 | PRR13 | CDRH1 | DFYMK |
| | 365 | PRR13 | CDRH2 | DIDPKNGDTFYNQKFKG |
| | 366 | PRR13 | CDRH3 | YYGYTMDY |
| | 367 | PRR13 | CDRL1 | TASSSVTSSYLH |
| | 368 | PRR13 | CDRL2 | STSNLAS |
| | 369 | PRR13 | CDRL3 | HQYRRPPRT |
| 255 | 370 | MW8 | CDRH1 | DYYMY |
| | 371 | MW8 | CDRH2 | TISDGGSYTYYPDNMKG |
| | 372 | MW8 | CDRH3 | DLGK |
| | 373 | MW8 | CDRL1 | TLSSQHSTYTIE |
| | 374 | MW8 | CDRL2 | LKKDGSHSTGD |
| | 375 | MW8 | CDRL3 | GVGDTIKEQFVYV |
| 256 | 376 | NI302.35C1 | CDRH1 | ITALS |
| | 377 | NI302.35C1 | CDRH2 | AITGNAYGTYYADSVKG |
| | 378 | NI302.35C1 | CDRH3 | GIASDSSGYSAF |
| | 379 | NI302.35C1 | CDRL1 | RASQSVDNQFA |
| | 380 | NI302.35C1 | CDRL2 | DASRRAP |
| | 381 | NI302.35C1 | CDRL3 | QHRYTWLYT |
| 257 | 382 | C6-17 | CDRH1 | EYTMH |
| | 383 | C6-17 | CDRH2 | GINPNNGGTRYNQKFKG |
| | 384 | C6-17 | CDRH3 | LDGRDY |
| | 385 | C6-17 | CDRL1 | KSSQSLLNSRTRKNYLA |
| | 386 | C6-17 | CDRL2 | WASTRES |
| | 387 | C6-17 | CDRL3 | KQSYNLLT |

TABLE 7
Amino acid sequences of the VHs of
exemplary huntingtin scFvs and nanobodies

| | SEQ | Huntingtin | |
|---|---|---|---|
| Construct | ID | scFv or | |
| ID | NO: | nanobody | VH amino acid sequence |
| 240 | 388 | C4 | QVQLQESGGGLVQPGGSLRLSCAASGFTFSSYSMSWVRQAPGKGLEW |
| | | | VAVISYDGSNKYYADSVKGRFTISRDNSKNTLYLQMNSLRAEDTAVY |
| | | | YCARDRYFDLWGRGTLVTVSS |
| 242 | 389 | NI-302.15E8 | EVQLVESGGGLIQPGGSLRLSCAVSGFTVSSYSMNWVRQAPGKGLEW |
| | | | VSYTSSSRSNTKKYADSVKGRFTISRDNARNSLYLQMNSLRDEDTAV |
| | | | YYCARAGDFGELLTGEGYYGMDVWGQGTTVTVSS |
| 243 | 390 | MW1 | QVKLQESGGGLVQPGGSLKLSCAASGFTFRDYYMYWVRQTPEKRLEW |
| | | | VAFISNGGGSTYYPDTVKGRFTISRDNAKNTLYLQMSRLKSEDTAMY |
| | | | YCARGRGYVWFAYWGQGTTVTVFS |
| 244 | 391 | MW2 | QVKLQESGPELKKPGETVKISCKASGYTFTNYGMNWVKQAPGKGLKW |
| | | | MGWINTYTGEPTYADDSKGRFAFSLETSASTAYLQINNLKNEDMATY |
| | | | FCARRGLLFAYWGQGTTVTVSS |
| 245 | 392 | 1C2 | SIQLVQSGPELKKPGETVRISCKASGYSFTTYGMNWVKQAPGKGLKW |
| | | | MGWINTYSGVPTYADDFKGRFAFSLETSASTAYLQINILKNEDTATY |
| | | | FCARRRSDGYSNYFDYWGQGSTLTVSS |
| 246 | 393 | 3B5H10 | QIQLVQSGPELKKPGETVKISCKASGYTFTTYGMSWVKQAPGKGFEW |
| | | | MGWINTYSGVPTYVDDFKGRFAFSLETSASTAYLQINNLKNEDTAVY |
| | | | FCARGGDNYLWFAYWGQGTLVTVSS |
| 247 | 394 | MW7 | QVKLQESGGGLVQPGGSMKLSCAASGFTFSDAWMDWVRQSPEKGLSG |
| | | | VAEIRSKANNHATYYAESVKGRFTISRDDSKSSVYLQMNSLRAEDTG |
| | | | IYYCIYAGFAYWGQGTTVTVSS |
| 248 | 395 | VHH2 | EVQLVESGGGLVQPGGSLRLSCAASGFSLDYYAIGWFRQAPGKEREG |
| | | | VSCISATDGSTYYADSVKGRFTISRDNAKNTVYLQMNSLKPEDTAVY |
| | | | YCATVRAPYSDYCNGYYDYWGQGTQVTVSS |
| 249 | 396 | VHH4 | EVQLVESGGGLVQPGGSLRLSCAASGFTLDYYAIGWFRQAPGKEREG |
| | | | VSCISSSDGSTYYADSVKGRFTISRDNAKNTVYLQMNSLKPEDTAVY |
| | | | YCATVRAPYSDYCNGYYDYWGQGTQVTVSS |
| 250 | 397 | NI-302.33C11 | EVQLVESGGGVVQPGNSLRLSCAASGFRFSDFGMHWVRQAPGKGLEW |
| | | | LALIWYDGGYKYYADSVKGRFTISRDNSKNTMFLQMNSLRAEDTAVY |
| | | | YCATHLEYCSRTTCYLGHWGQGTLVTVSS |
| 252 | 398 | INT41 | EVQLVVSGGGLVKPGGSMILSCAASGFTFSNYSMNWVRQAPGKGLEW |
| | | | VSSISSSSEYIYYADFVKGRFTISRDNAKNSLYLQMDSLRAEDTAVY |
| | | | YCAWPGYRKAWGRGTLVTVSS |
| 253 | 399 | NI302.63F3 | QVQLVQSGSAFKKPGTSVKVSCKASGYTFETRSMNWVRQAPGQGLEY |
| | | | MGWINTNTGNRTYVQAFRGRFVFSLDTSVSTAYLQISNLKTEDTAVY |
| | | | YCARGAGGGYWFDSWGQGTLVTVSS |
| 254 | 400 | PRR13 | EVQLVESGPEVKKPGATVKISCKVSGYTFTDFYMKWVQQAPGRGLEW |
| | | | MGDIDPKNGDTFYNQKFKGRVTMTADTSTGTAYMQLSSLTSEDTAVY |
| | | | FCASYYGYTMDYWGQGTTVTVAS |
| 255 | 401 | MW8 | QVQLQESGGGLVKPGGSLKLSCAASGFTFSDYYMYWVRQTPEKRLEW |
| | | | VATISDGGSYTYYPDNMKGRFTISRDNAKNNLYLQMSSLKSEDTAMY |
| | | | FCARDLGKWGQGTTVTVSS |
| 256 | 402 | NI302.35C1 | EVQLVESGGNLVQPGGSLRLSCTASGFTFSITALSWVRQAPEKGPQW |
| | | | VSAITGNAYGTYYADSVKGRFTISRDNAKNTLYLQMNGLRAEDTAIY |
| | | | YCVKGIASDSSGYSAFWGPGTLVTVSS |
| 257 | 403 | C6-17 | QVQLVQSGAEVKKPGASVKVSCKASGYTFTEYTMHWVRQAPGRGLEW |
| | | | MGGINPNNGGTRYNQKFKGRVTMTRDTSIRTAYVELSRLTSDDTAVY |
| | | | YCASLDGRDYWGQGTLVTVSS |

TABLE 8
Amino acid sequences of the VLs of
exemplary huntingtin scFvs and nanobodies

| | SEQ | Huntingtin | |
|---|---|---|---|
| Construct | ID | scFv or | |
| ID | NO: | nanobody | VL amino acid sequence |
| 240 | 404 | C4 | QSALTQPASVSGSPGQSITISCTGTSSDIGAYNYVSWYQQYPGKAPKLL |
| | | | IYDVSNRPSGISNRFSGSKSGDTASLTISGLQAEDEADYYCSSFANSGP |
| | | | LFGGGTKVTVLG |
| 241 | 405 | VL12.3 | QPVLTQSPSVSAAPRQRVTISVSGSNSNIGSNTVNWIQQLPGRAPELLM |
| | | | YDDDLLAPGVSDRFSGSRSGTSASLTISGLQSEDEADYYAATWDDSLNG |
| | | | WVFGGGTKVTVLS |
| 242 | 406 | NI-302.15E8 | SYELTQPPSVSVSPGQTATITCSGDELGDKYVGWYQQKPGQSPLLVIYQ |
| | | | DAKRPSGIPERFSGSNSGNTATLTISGTQAMDEADYYCQAWDSGTMVFG |
| | | | GGTRLTVL |
| 243 | 407 | MW1 | QLVLTQSSSASFSLGASAKLTCTLSSQHSTYTIEWYQQQPLKPPKYVME |
| | | | LKKDGSHSTGDGIPDRFSGSSSGADRYLSISNIQPEDEAIYICGVGDTI |
| | | | KEQFVYVFGGGTKVTVLG |
| 244 | 408 | MW2 | QLVLTQSSSASFSLGASAKLTCTLSSQHSTYTIEWYQQQPLKPPKYVME |
| | | | LKKDGSHSTGDGIPDRFSGSSSGADRYLSISNIQPEDEAIYICGVGDTI |
| | | | KEQFVYVFGGGTKVTVLG |
| 245 | 409 | 1C2 | QLVLTQSSSASFSLGASAKLTCTLSRQHSTYTIEWYQQQPLKPPKFVME |
| | | | LKKDGSHSTGDGIPDRFSGSSSGAHRYLSISNIQPEDEAIYICGVGDTI |
| | | | KEQFVYVFGGGTKVTVLG |
| 246 | 410 | 3B5H10 | QLVLTQSSSASFSLGASAKLTCTLNSQHSTYTIEWYQQQPLKPPKYVME |
| | | | LKKDGSHSTGDGIPDRFSGSSSGADRYLSISNIQPEDEAIYICGVGDTI |
| | | | KEQFVYVFGGGTKVTVLG |
| 247 | 411 | MW7 | DIELTQSPSSLAMSVGQKVTMSCKSSQSLLNSSNQKNYLAWYQQKPGQS |
| | | | PKLLVYFASTRESGVPDRFIGSGSGTDFTLTISSVQAEDLADYFCQQHY |
| | | | STPWTFGGGTKLEIKR |
| 250 | 412 | NI-302.33C11 | DIQLTQSPSFLSASVGDTVTFTCRASQGISDYLAWFQQKPGIAPKLLIY |
| | | | AASTLQTGVPSRFSGSGSGTEFTLTIRSLQSEDFGTYYCQQLKTYPYTF |
| | | | GQGTKVEIKS |
| 251 | 413 | Happ1 | QSVLTQPPSASGTPGQRVTISCSGSSSNIGSNYVYWYQQLPGTAPKLLI |
| | | | YRNNQRPSGVPDRFSGSKSGTSASLAISGLRPEDEADYYCAAWDDSLCV |
| | | | ALVFGGGTNGGGGVDGTAG |
| 252 | 414 | INT41 | QSVLTQPASVSGSPGQSITISCAGTSSDVGGYNYVSWYQQHPGKAPKLM |
| | | | IYEDSKRPSGVSNRFSGSKSGNTASLTISGLRAEDEADYYCSYCASKGH |
| | | | WLFGGGTKLAVLGAAAEQKLIS |
| 253 | 415 | NI302.63F3 | DIQMTQSPDSLAVSLGERATINCKSNQSLFYSSNNNNYLAWYQHKSGQP |
| | | | PKLLVYWGSTRESGVPDRFSGSGSGTDFTLTISSLQAEDVAIYYCHQYY |
| | | | HNPYTFGQGTKLEIKR |
| 254 | 416 | PRR13 | EIVLTQSPSSLSASVGDRVTITCTASSSVTSSYLHWYQQKPGKAPKLLI |
| | | | YSTSNLASGVPSRFSGSGSGTDFTFTISSLQPEDIATYYCHQYRRPPRT |
| | | | FGGGTKLEIKR |
| 255 | 417 | MW8 | QLVLTQSSSASFSLGASAKLTCTLSSQHSTYTIEWYQQQPLKPPKYVME |
| | | | LKKDGSHSTGDGIPDRFSGSSSGADRYLSISNIQPEDEAIYICGVGDTI |
| | | | KEQFVYVFGGGTKVTVLG |
| 256 | 418 | NI302.35C1 | EIVLTQSPATLSLSPGERATLSCRASQSVDNQFAWYQQKPGQAPRLLIY |
| | | | DASRRAPGIPDRFSGSGSGTDFTLTISSLEPEDFAIYYCQHRYTWLYTF |
| | | | GQGTRLEIKR |
| 257 | 419 | C6-17 | QSPDSLAVSLGERATINCKSSQSLLNSRTRKNYLAWYQQKPGQPPKLLI |
| | | | YWASTRESGVPDRFSGSGSGTDFTLTISSLQAEDVAVYYCKQSYNLLTF |
| | | | GGGTKLEIKR |

In an embodiment, the scFv comprises a heavy chain variable domain comprising the CDRH1, CDRH2, and CDRH3 amino acid sequences of SEQ ID NOs: 292, 293, and 294; 301, 302, and 303; 307, 308, and 309; 313, 314, and 315; 319, 320, and 321; 325, 326, and 327; 331, 332, and 333; 343, 344, and 345; 352, 353, and 354; 358, 359, and 360; 364, 365, and 366; 370, 371, and 372; 376, 377, and 378; or 382, 383, and 384, respectively.

In an embodiment, the scFv comprises a light chain variable domain comprising the CDRL1, CDRL2, and CDRL3 amino acid sequences of SEQ ID NOs: 295, 296, and 297; 304, 305, and 306; 310, 311, and 312; 316, 317, and 318; 322, 323, and 324; 328, 329, and 330; 334, 335, and 336; 346, 347, and 348; 355, 356, and 357; 361, 362, and 363; 367, 368, and 369; 373, 374, and 375; 379, 380, and 381; or 385, 386, and 387, respectively.

In an embodiment, the scFv comprises a heavy chain variable domain and a light chain variable domain comprising the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the scFv comprises a heavy chain variable domain comprising the amino acid sequence of SEQ ID NOs: 388-394 or 397-399; or a light chain variable domain comprising the amino acid sequence of SEQ ID NOs: 404, 406-412, 414, or 415.

In an embodiment, the heavy chain variable domain and light chain variable domain comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the nanobody comprises a heavy chain variable domain comprising the CDRH1, CDRH2, and CDRH3 amino acid sequences of SEQ ID NOs: 337, 338, and 339; or 340, 341, and 342, respectively; or a light chain variable domain comprising the CDRL1, CDRL2, and CDRL3 amino acid sequences of SEQ ID NOs: 298, 299, and 300; or 349, 350, and 351, respectively.

In an embodiment, the heavy chain variable domain comprises the amino acid sequences of SEQ ID NOs: 395 or 396; or the light chain variable domain comprises the amino acid sequences of SEQ ID NOs: 405 or 413.

In an embodiment, the chimeric molecule comprises an scFv or nanobody that specifically binds to human huntingtin and a degron comprising an amino acid sequence selected from the group consisting of SEQ ID NOs: 1-17 and 20.

In an embodiment, the chimeric molecule comprises an scFv that specifically binds to human huntingtin and a degron comprising the amino acid sequence of SEQ ID NO: 1, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 2, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306;
307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 3, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 4, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 5, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 6, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 7, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 8, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 9, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 10, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 11, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 12, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 13, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 14, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 15, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 16, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 17, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 20, wherein the scFv comprises the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387, respectively.

In an embodiment, the chimeric molecule comprises an scFv that specifically binds to human huntingtin and a degron comprising the amino acid sequence of SEQ ID NO: 1, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 2, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 3, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 4, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 5, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 6, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 7, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 8, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 9, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 10, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 11, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 12, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 13, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 14, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 15, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 16, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 17, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the chimeric molecule comprises an scFv that specifically binds to human huntingtin and a degron comprising the amino acid sequence of SEQ ID NO: 20, wherein the heavy chain variable domain and light chain variable domain of the scFv comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415, respectively.

In an embodiment, the polypeptide is an scFv that specifically binds to human polyglutamine (polyQ) protein. In an embodiment, the polypeptide is an scFv that specifically binds to human alpha-synuclein. In an embodiment, the polypeptide is an scFv that specifically binds to human tau protein. In an embodiment, the polypeptide is an scFv that specifically binds to human amyloid beta. In an embodiment, the polypeptide is an scFv that specifically binds to human lamin. In an embodiment, the polypeptide is an scFv that specifically binds to human phospholamban (PLN) protein.

### Polynucleotides, Vectors, and Methods of Producing Chimeric Molecules

In an aspect, provided herein is an isolated polynucleotide encoding a chimeric molecule protein disclosed herein.

In an embodiment, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid. Such methods can increase expression of the encoded chimeric molecule relative to the expression of the chimeric molecule encoded by polynucleotides that have not been optimized.

In an aspect, provided herein is a vector comprising a polynucleotide disclosed herein. Suitable vectors, include, without limitation, plasmids, viruses, cosmids, artificial chromosomes, linear DNA, and mRNA. In an embodiment, the vector is a plasmid or a viral vector. In an embodiment, the vector is a retrovirus vector, a herpes virus vector, a baculovirus vector, or an adenovirus vector. In an embodiment, the vector is an expression vector.

Vectors (e.g., expression vectors) can be introduced into cells (using any techniques known in the art) for propagation of the vector and/or for expression of a chimeric molecule encoded by the vector. Accordingly, in another aspect, the instant disclosure provides a recombinant cell comprising a polynucleotide or a vector (e.g., an expression vector) disclosed herein. In another aspect, the instant disclosure provides a method of producing a chimeric molecule, the method comprising culturing the recombinant cell under conditions whereby the polynucleotide is expressed, and the chimeric molecule is produced.

A variety of host cells and expression vector systems can be utilized to express the chimeric molecules described herein. In these expression systems, the coding sequences of interest can be produced and subsequently purified. These expression systems also represent cells which can, when transformed or transfected with the appropriate nucleotide coding sequences, express a chimeric molecule described herein in situ. These include but are not limited to microorganisms such as bacteria (e.g., E. coli and B. subtilis) transformed with, e.g., recombinant bacteriophage DNA, plasmid DNA, or cosmid DNA expression vectors containing chimeric molecule coding sequences; yeast (e.g., Saccharomyces Pichia) transformed with, e.g., recombinant yeast expression vectors containing chimeric molecule coding sequences; insect cell systems infected with, e.g., recombinant virus expression vectors (e.g., baculovirus) containing chimeric molecule coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing chimeric molecule coding sequences; or mammalian cell systems (e.g., COS (e.g., COSI or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing the chimeric molecules described herein are human cells, e.g., human cell lines. In an embodiment, a mammalian expression vector is pOptiVEC™ or pcDNA3.3. In an embodiment, bacterial cells such as Escherichia coli, or eukaryotic cells (e.g., mammalian cells), are used for the expression of a chimeric molecule. For example, mammalian cells such as CHO or HEK293 cells, in conjunction with a vector such as the major intermediate early gene promoter element from human cytomegalovirus is an effective expression system for chimeric molecules disclosed herein. In an embodiment, insect cells (e.g., Sf9 cells) are used for the expression of a chimeric molecule.

In an insect system, Autographa californica nuclear polyhedrosis virus (AcNPV), for example, can be used as a vector to express foreign genes. The virus grows in Spodoptera frugiperda cells. The capsid protein coding sequence can be cloned individually into non-essential regions (for example the polyhedrin gene) of the virus and placed under control of an AcNPV promoter (for example the polyhedrin promoter).

For long-term, high-yield production of recombinant proteins, stable expression cells can be generated. For example, cell lines which stably express a capsid protein described herein can be engineered.

In an embodiment, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a chimeric molecule described herein or a fragment thereof.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; Morgan R A & Anderson W F (1993) Ann Rev Biochem 62:191-217; and Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

### AAV Compositions

In an aspect, provided herein is a recombinant adeno associated virus (rAAV) comprising a capsid and a viral genome, wherein the viral genome comprises at least one inverted terminal repeat (ITR) region and a polynucleotide encoding a chimeric molecule disclosed herein.

In an embodiment, the capsid comprises a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof.

In an embodiment, the capsid protein comprises an amino acid sequence selected from the group consisting of SEQ ID NO: 258-289.

TABLE 9
Amino acid sequences of AAV capsid proteins

| | SEQ ID | |
|---|---|---|
| Description | NO | Amino acid sequence |
| AAV5 (no peptide | 258 | MSFVDHPPDWLEEVGEGLREFLGLEAGPPKPKPNQQHQDQARGLV |
| insertion) | | LPGYNYLGPGNGLDRGEPVNRADEVAREHDISYNEQLEAGDNPYL |
| | | KYNHADAEFQEKLADDTSFGGNLGKAVFQAKKRVLEPFGLVEEGA |
| | | KTAPTGKRIDDHFPKRKKARTEEDSKPSTSSDAEAGPSGSQQLQIPA |
| | | QPASSLGADTMSAGGGGPLGDNNQGADGVGNASGDWHCDSTWM |
| | | GDRVVTKSTRTWVLPSYNNHQYREIKSGSVDGSNANAYFGYSTPW |
| | | GYFDFNRFHSHWSPRDWQRLINNYWGFRPRSLRVKIFNIQVKEVTV |
| | | QDSTTTIANNLTSTVQVFTDDDYQLPYVVGNGTEGCLPAFPPQVFT |
| | | LPQYGYATLNRDNTENPTERSSFFCLEYFPSKMLRTGNNFEFTYNFE |
| | | EVPFHSSFAPSQNLFKLANPLVDQYLYRFVSTNNTGGVQFNKNLAG |
| | | RYANTYKNWFPGPMGRTQGWNLGSGVNRASVSAFATTNRMELEG |
| | | ASYQVPPQPNGMTNNLQGSNTYALENTMIFNSQPANPGTTATYLE |
| | | GNMLITSESETQPVNRVAYNVGGQMATNNQSSTTAPATGTYNLQEI |
| | | VPGSVWMERDVYLQGPIWAKIPETGAHFHPSPAMGGFGLKHPPPM |
| | | MLIKNTPVPGNITSFSDVPVSSFITQYSTGQVTVEMEWELKKENSKR |
| | | WNPEIQYTNNYNDPQFVDFAPDSTGEYRTTRPIGTRYLTRPL |
| AAV5.2 (no peptide | 259 | MAADGYLPDWLEDTLSEGIRQWWKLKPGPPPPKPAERHKDDSRGL |
| insertion) | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTTAPATGTYN |
| | | LQEIVPGSVWMERDVYLQGPIWAKIPETGAHFHPSPAMGGFGLKHP |
| | | PPMMLIKNTPVPGNITSFSDVPVSSFITQYSTGQVTVEMEWELKKEN |
| | | SKRWNPEIQYTNNYNDPQFVDFAPDSTGEYRTTRPIGTRYLTRPL |
| VYPep1, loop IV | 260 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNASLEEEWAQ |
| | | VECEVYGRGCPSGSLDESFYDWFERQLGATGGVQFNKNLAGRYAN |
| | | TYKNWFPGPMGRTQGWNLGSGVNRASVSAFATTNRMELEGASYQ |
| | | VPPQPNGMTNNLQGSNTYALENTMIFNSQPANPGTTATYLEGNMLI |
| | | TSESETQPVNRVAYNVGGQMATNNQSSTTAPATGTYNLQEIVPGSV |
| | | WMERDVYLQGPIWAKIPETGAHFHPSPAMGGFGLKHPPPMMLIKN |
| | | TPVPGNITSFSDVPVSSFITQYSTGQVTVEMEWELKKENSKRWNPEI |
| | | QYTNNYNDPQFVDFAPDSTGEYRTTRPIGTRYLTRPL |
| VYPep1, loop VIII | 261 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSASLEEEWAQV |
| | | ECEVYGRGCPSGSLDESFYDWFERQLGATTAPATGTYNLQEIVPGS |
| | | VWMERDVYLQGPIWAKIPETGAHFHPSPAMGGFGLKHPPPMMLIK |
| | | NTPVPGNITSFSDVPVSSFITQYSTGQVTVEMEWELKKENSKRWNP |
| | | EIQYTNNYNDPQFVDFAPDSTGEYRTTRPIGTRYLTRPL |
| VYPep7, loop IV | 262 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNAPLTNTVKA |
| | | TGGVQFNKNLAGRYANTYKNWFPGPMGRTQGWNLGSGVNRASV |
| | | SAFATTNRMELEGASYQVPPQPNGMTNNLQGSNTYALENTMIFNS |
| | | QPANPGTTATYLEGNMLITSESETQPVNRVAYNVGGQMATNNQSS |
| | | TTAPATGTYNLQEIVPGSVWMERDVYLQGPIWAKIPETGAHFHPSP |
| | | AMGGFGLKHPPPMMLIKNTPVPGNITSFSDVPVSSFITQYSTGQVTV |
| | | EMEWELKKENSKRWNPEIQYTNNYNDPQFVDFAPDSTGEYRTTRPI |
| | | GTRYLTRPL |
| VYPep7, loop VIII | 263 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSAPLTNTVKAT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep8, loop IV | 264 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNDDTRHWGTG |
| | | GVQFNKNLAGRYANTYKNWFPGPMGRTQGWNLGSGVNRASVSA |
| | | FATTNRMELEGASYQVPPQPNGMTNNLQGSNTYALENTMIFNSQP |
| | | ANPGTTATYLEGNMLITSESETQPVNRVAYNVGGQMATNNQSSTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep8, loop VIII | 265 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSDDTRHWGTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep9, loop IV | 266 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNEYHHYNKTG |
| | | GVQFNKNLAGRYANTYKNWFPGPMGRTQGWNLGSGVNRASVSA |
| | | FATTNRMELEGASYQVPPQPNGMTNNLQGSNTYALENTMIFNSQP |
| | | ANPGTTATYLEGNMLITSESETQPVNRVAYNVGGQMATNNQSSTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep9, loop VIII | 267 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSEYHHYNKTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep10, loop IV | 268 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNQLFPLFRTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep10, loop VIII | 269 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSQLFPLFRTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep12, loop IV | 270 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFNPVTGEVPP |
| | | RYPLDARTGGVQFNKNLAGRYANTYKNWFPGPMGRTQGWNLGS |
| | | GVNRASVSAFATTNRMELEGASYQVPPQPNGMTNNLQGSNTYALE |
| | | NTMIFNSQPANPGTTATYLEGNMLITSESETQPVNRVAYNVGGQM |
| | | ATNNQSSTTAPATGTYNLQEIVPGSVWMERDVYLQGPIWAKIPETG |
| | | AHFHPSPAMGGFGLKHPPPMMLIKNTPVPGNITSFSDVPVSSFITQY |
| | | STGQVTVEMEWELKKENSKRWNPEIQYTNNYNDPQFVDFAPDSTG |
| | | EYRTTRPIGTRYLTRPL |
| VYPep12, loop VIII | 271 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFNPVTGEVPP |
| | | RYPLDARTTAPATGTYNLQEIVPGSVWMERDVYLQGPIWAKIPETG |
| | | AHFHPSPAMGGFGLKHPPPMMLIKNTPVPGNITSFSDVPVSSFITQY |
| | | STGQVTVEMEWELKKENSKRWNPEIQYTNNYNDPQFVDFAPDSTG |
| | | EYRTTRPIGTRYLTRPL |
| VYPep13, loop IV | 272 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNSVTEQGAELS |
| | | NEERTGGVQFNKNLAGRYANTYKNWFPGPMGRTQGWNLGSGVN |
| | | RASVSAFATTNRMELEGASYQVPPQPNGMTNNLQGSNTYALENTM |
| | | IFNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep13, loop VIII | 273 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSSVTEQGAELS |
| | | NEERTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |
| VYPep14, loop IV | 274 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGQSGRGDLG |
| | | LTGGVQFNKNLAGRYANTYKNWFPGPMGRTQGWNLGSGVNRAS |
| | | VSAFATTNRMELEGASYQVPPQPNGMTNNLQGSNTYALENTMIFN |
| | | SQPANPGTTATYLEGNMLITSESETQPVNRVAYNVGGQMATNNQS |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPep14, loop VIII | 275 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGQSGRGDLGL |
| | | TTAPATGTYNLQEIVPGSVWMERDVYLQGPIWAKIPETGAHFHPSP |
| | | AMGGFGLKHPPPMMLIKNTPVPGNITSFSDVPVSSFITQYSTGQVTV |
| | | EMEWELKKENSKRWNPEIQYTNNYNDPQFVDFAPDSTGEYRTTRPI |
| | | GTRYLTRPL |
| VYPep15, loop IV | 276 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGASSLNIAG |
| | | LSTGGVQFNKNLAGRYANTYKNWFPGPMGRTQGWNLGSGVNRAS |
| | | VSAFATTNRMELEGASYQVPPQPNGMTNNLQGSNTYALENTMIFN |
| | | SQPANPGTTATYLEGNMLITSESETQPVNRVAYNVGGQMATNNQS |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPep15, loop VIII | 277 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTGASSLNIAGL |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPep2, loop IV | 278 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHKAKGPR |
| | | KLGTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep2, loop VIII | 279 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHKAKGPR |
| | | KLGTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPcp3, loop IV | 280 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHAIYPRHT |
| | | GGVQFNKNLAGRYANTYKNWFPGPMGRTQGWNLGSGVNRASVS |
| | | AFATTNRMELEGASYQVPPQPNGMTNNLQGSNTYALENTMIFNSQ |
| | | PANPGTTATYLEGNMLITSESETQPVNRVAYNVGGQMATNNQSST |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep3, loop VIII | 281 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHAIYPRHT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep4, loop IV | 282 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNRTIGPSVTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep4, loop VIII | 283 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSRTIGPSVTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop IV | 284 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNLSSRLDATGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop VIII | 285 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSLSSRLDATTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep6, loop IV | 286 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTHRPPMWSP |
| | | VWPTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep6, loop VIII | 287 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTHRPPMWSPV |
| | | WPTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop IV | 288 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFGQSSLPRDG |
| | | PNTGGVQFNKNLAGRYANTYKNWFPGPMGRTQGWNLGSGVNRA |
| | | SVSAFATTNRMELEGASYQVPPQPNGMTNNLQGSNTYALENTMIF |
| | | NSQPANPGTTATYLEGNMLITSESETQPVNRVAYNVGGQMATNNQ |
| | | SSTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop VIII | 289 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFGQSSLPRDGP |
| | | NGGTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |

Additional embodiments of the recombinant AAV capsid proteins disclosed herein are listed in Table S1 below.

TABLE S1
Amino acid sequences of additional recombinant AAV capsid proteins

| | SEQ ID | |
|---|---|---|
| Description | NO | Amino acid sequence |
| AAV5.2 (no peptide | 420 | TAADGYLPDWLEDTLSEGIRQWWKLKPGPPPPKPAERHKDDSRGL |
| insertion) | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTTAPATGTYN |
| | | LQEIVPGSVWMERDVYLQGPIWAKIPETGAHFHPSPAMGGFGLKHP |
| | | PPMMLIKNTPVPGNITSFSDVPVSSFITQYSTGQVTVEMEWELKKEN |
| | | SKRWNPEIQYTNNYNDPQFVDFAPDSTGEYRTTRPIGTRYLTRPL |
| VYPep1, loop IV | 421 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNASLEEEWAQ |
| | | VECEVYGRGCPSGSLDESFYDWFERQLGATGGVQFNKNLAGRYAN |
| | | TYKNWFPGPMGRTQGWNLGSGVNRASVSAFATTNRMELEGASYQ |
| | | VPPQPNGMTNNLQGSNTYALENTMIFNSQPANPGTTATYLEGNMLI |
| | | TSESETQPVNRVAYNVGGQMATNNQSSTTAPATGTYNLQEIVPGSV |
| | | WMERDVYLQGPIWAKIPETGAHFHPSPAMGGFGLKHPPPMMLIKN |
| | | TPVPGNITSFSDVPVSSFITQYSTGQVTVEMEWELKKENSKRWNPEI |
| | | QYTNNYNDPQFVDFAPDSTGEYRTTRPIGTRYLTRPL |
| VYPep1, loop VIII | 422 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSASLEEEWAQV |
| | | ECEVYGRGCPSGSLDESFYDWFERQLGATTAPATGTYNLQEIVPGS |
| | | VWMERDVYLQGPIWAKIPETGAHFHPSPAMGGFGLKHPPPMMLIK |
| | | NTPVPGNITSFSDVPVSSFITQYSTGQVTVEMEWELKKENSKRWNP |
| | | EIQYTNNYNDPQFVDFAPDSTGEYRTTRPIGTRYLTRPL |
| VYPep7, loop IV | 423 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNAPLTNTVKA |
| | | TGGVQFNKNLAGRYANTYKNWFPGPMGRTQGWNLGSGVNRASV |
| | | SAFATTNRMELEGASYQVPPQPNGMTNNLQGSNTYALENTMIFNS |
| | | QPANPGTTATYLEGNMLITSESETQPVNRVAYNVGGQMATNNQSS |
| | | TTAPATGTYNLQEIVPGSVWMERDVYLQGPIWAKIPETGAHFHPSP |
| | | AMGGFGLKHPPPMMLIKNTPVPGNITSFSDVPVSSFITQYSTGQVTV |
| | | EMEWELKKENSKRWNPEIQYTNNYNDPQFVDFAPDSTGEYRTTRPI |
| | | GTRYLTRPL |
| VYPcp7, loop VIII | 424 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSAPLTNTVKAT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep8, loop IV | 425 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNDDTRHWGTG |
| | | GVQFNKNLAGRYANTYKNWFPGPMGRTQGWNLGSGVNRASVSA |
| | | FATTNRMELEGASYQVPPQPNGMTNNLQGSNTYALENTMIFNSQP |
| | | ANPGTTATYLEGNMLITSESETQPVNRVAYNVGGQMATNNQSSTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep8, loop VIII | 426 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSDDTRHWGTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep9, loop IV | 427 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNEYHHYNKTG |
| | | GVQFNKNLAGRYANTYKNWFPGPMGRTQGWNLGSGVNRASVSA |
| | | FATTNRMELEGASYQVPPQPNGMTNNLQGSNTYALENTMIFNSQP |
| | | ANPGTTATYLEGNMLITSESETQPVNRVAYNVGGQMATNNQSSTT |
| | | APATGTYNLQEIVPGSVWMERDVYLQGPIWAKIPETGAHFHPSPAM |
| | | GGFGLKHPPPMMLIKNTPVPGNITSFSDVPVSSFITQYSTGQVTVEM |
| | | EWELKKENSKRWNPEIQYTNNYNDPQFVDFAPDSTGEYRTTRPIGT |
| | | RYLTRPL |
| VYPep9, loop VIII | 428 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSEYHHYNKTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep10, loop IV | 429 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNQLFPLFRTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep10, loop VIII | 430 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSQLFPLFRTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep12, loop IV | 431 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFNPVTGEVPP |
| | | RYPLDARTGGVQFNKNLAGRYANTYKNWFPGPMGRTQGWNLGS |
| | | GVNRASVSAFATTNRMELEGASYQVPPQPNGMTNNLQGSNTYALE |
| | | NTMIFNSQPANPGTTATYLEGNMLITSESETQPVNRVAYNVGGQM |
| | | ATNNQSSTTAPATGTYNLQEIVPGSVWMERDVYLQGPIWAKIPETG |
| | | AHFHPSPAMGGFGLKHPPPMMLIKNTPVPGNITSFSDVPVSSFITQY |
| | | STGQVTVEMEWELKKENSKRWNPEIQYTNNYNDPQFVDFAPDSTG |
| | | EYRTTRPIGTRYLTRPL |
| VYPep12, loop VIII | 432 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFNPVTGEVPP |
| | | RYPLDARTTAPATGTYNLQEIVPGSVWMERDVYLQGPIWAKIPETG |
| | | AHFHPSPAMGGFGLKHPPPMMLIKNTPVPGNITSFSDVPVSSFITQY |
| | | STGQVTVEMEWELKKENSKRWNPEIQYTNNYNDPQFVDFAPDSTG |
| | | EYRTTRPIGTRYLTRPL |
| VYPep13, loop IV | 433 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNSVTEQGAELS |
| | | NEERTGGVQFNKNLAGRYANTYKNWFPGPMGRTQGWNLGSGVN |
| | | RASVSAFATTNRMELEGASYQVPPQPNGMTNNLQGSNTYALENTM |
| | | IFNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep13, loop VIII | 434 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSSVTEQGAELS |
| | | NEERTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |
| VYPep14, loop IV | 435 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGQSGRGDLG |
| | | LTGGVQFNKNLAGRYANTYKNWFPGPMGRTQGWNLGSGVNRAS |
| | | VSAFATTNRMELEGASYQVPPQPNGMTNNLQGSNTYALENTMIFN |
| | | SQPANPGTTATYLEGNMLITSESETQPVNRVAYNVGGQMATNNQS |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPep14, loop VIII | 436 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGQSGRGDLGL |
| | | TTAPATGTYNLQEIVPGSVWMERDVYLQGPIWAKIPETGAHFHPSP |
| | | AMGGFGLKHPPPMMLIKNTPVPGNITSFSDVPVSSFITQYSTGQVTV |
| | | EMEWELKKENSKRWNPEIQYTNNYNDPQFVDFAPDSTGEYRTTRPI |
| | | GTRYLTRPL |
| VYPep15, loop IV | 437 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGASSLNIAG |
| | | LSTGGVQFNKNLAGRYANTYKNWFPGPMGRTQGWNLGSGVNRAS |
| | | VSAFATTNRMELEGASYQVPPQPNGMTNNLQGSNTYALENTMIFN |
| | | SQPANPGTTATYLEGNMLITSESETQPVNRVAYNVGGQMATNNQS |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPep15, loop VIII | 438 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTGASSLNIAGL |
| | | STTAPATGTYNLQEIVPGSVWMERDVYLQGPIWAKIPETGAHFHPS |
| | | PAMGGFGLKHPPPMMLIKNTPVPGNITSFSDVPVSSFITQYSTGQVT |
| | | VEMEWELKKENSKRWNPEIQYTNNYNDPQFVDFAPDSTGEYRTTR |
| | | PIGTRYLTRPL |
| VYPcp2, loop IV | 439 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHKAKGPR |
| | | KLGTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep2, loop VIII | 440 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHKAKGPR |
| | | KLGTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep3, loop IV | 441 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHAIYPRHT |
| | | GGVQFNKNLAGRYANTYKNWFPGPMGRTQGWNLGSGVNRASVS |
| | | AFATTNRMELEGASYQVPPQPNGMTNNLQGSNTYALENTMIFNSQ |
| | | PANPGTTATYLEGNMLITSESETQPVNRVAYNVGGQMATNNQSST |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep3, loop VIII | 442 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHAIYPRHT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep4, loop IV | 443 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNRTIGPSVTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep4, loop VIII | 444 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSRTIGPSVTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop IV | 445 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNLSSRLDATGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop VIII | 446 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSLSSRLDATTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep6, loop IV | 447 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTHRPPMWSP |
| | | VWPTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep6, loop VIII | 448 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTHRPPMWSPV |
| | | WPTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop IV | 449 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFGQSSLPRDG |
| | | PNTGGVQFNKNLAGRYANTYKNWFPGPMGRTQGWNLGSGVNRA |
| | | SVSAFATTNRMELEGASYQVPPQPNGMTNNLQGSNTYALENTMIF |
| | | NSQPANPGTTATYLEGNMLITSESETQPVNRVAYNVGGQMATNNQ |
| | | SSTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop VIII | 450 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFGQSSLPRDGP |
| | | NGGTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |

In an embodiment, the capsid protein comprises an amino acid sequence selected from the group consisting of SEQ ID NO: 420-450.

In an embodiment, the recombinant AAV capsid protein comprises an amino acid sequence selected from the group consisting of SEQ ID NOs: 258-289 and 420-450. In an embodiment, the amino acid sequence of the recombinant AAV capsid protein consists of an amino acid sequence selected from the group consisting of SEQ ID NOs: 258-289 and 420-450.

In an aspect, provided herein is a pharmaceutical composition comprising an rAAV disclosed herein, and a pharmaceutically acceptable carrier.

The rAAVs disclosed herein generally comprise a recombinant genome (e.g., an rAAV genome) packaged within the capsid. The rAAV genome can be of any type that is capable of being packaged within an AAV capsid disclosed herein. For example, in an embodiment, the rAAV genome is a single-stranded DNA genome. In an embodiment, the rAAV genome is a self-complementary genome, for example as described in U.S. Pat. No. 7,790,154, which is hereby incorporated by reference in its entirety.

In an embodiment, the rAAV genome comprises a transgene. In an embodiment, the transgene encodes a therapeutic protein. In an embodiment, the transgene encodes an antibody or a fragment thereof (e.g., a Fab, scFv, or full-length antibody). In an embodiment, the transgene encodes an scFv, nanobody, or VHH. In an embodiment, the transgene encodes a non-coding RNA.

In an embodiment, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In an embodiment, the rAAV genome comprises a TRE operably linked to the transgene, to control expression of an RNA or polypeptide encoded by the transgene. In an embodiment, the TRE comprises a constitutive promoter. In an embodiment, the TRE is active in any mammalian cell (e.g., any human cell). In an embodiment, the TRE is active in a broad range of human cells. Such TREs may comprise constitutive promoter and/or enhancer elements, including any of those described herein, and any of those known to one of skill in the art. In an embodiment, the TRE comprises an inducible promoter. In an embodiment, the TRE is a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. Tissue-specific promoter and/or enhancer elements can be isolated from genes specifically expressed in the tissue by methods well known in the art.

Suitable promoters include, e.g., cytomegalovirus promoter (CMV) (Stinski et al., (1985) Journal of Virology 55 (2): 431-441), CMV early enhancer/chicken β-actin (CBA) promoter/rabbit β-globin intron (CAG) (Miyazaki et al., (1989) Gene 79 (2): 269-277), hybrid form of the CBA promoter (CBh) (Gray et al., (2011) Hum Gene Ther. 22 (9): 1143-53), CBSB (Jacobson et al., (2006) Molecular Therapy 13 (6): 1074-1084), human elongation factor 1α promoter (EF1α) (Kim et al., (1990) Gene 91 (2): 217-223), human phosphoglycerate kinase promoter (PGK) (Singer-Sam et al., (1984) Gene 32 (3): 409-417), mitochondrial heavy-strand promoter (Lodeiro et al., (2012) PNAS 109 (17): 6513-6518), ubiquitin promoter (Wulff et al., (1990) FEBS Letters 261:101-105).

In an aspect, provided herein is a pharmaceutical composition comprising an rAAV disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed., Amer. Pharmaceutical Assoc.

### Methods of Use

In an aspect, provided herein is a method for reducing the level of human TDP-43 in a cell, the method comprising introducing into the cell a chimeric molecule disclosed herein, a pharmaceutical composition disclosed herein, a polynucleotide disclosed herein, an expression vector disclosed herein, or an rAAV disclosed herein.

In an aspect, provided herein is a method for reducing the level of human huntingtin in a cell, the method comprising introducing into the cell a chimeric molecule disclosed herein, a pharmaceutical composition disclosed herein, a polynucleotide disclosed herein, an expression vector disclosed herein, or an rAAV disclosed herein.

In an aspect, provided herein is a method for reducing the level of polyglutamine (PolyQ) protein, alpha-synuclein, tau protein, amyloid beta, lamin, or phospholamban (PLN) protein in a cell, the method comprising introducing into the cell a chimeric molecule disclosed herein, a pharmaceutical composition disclosed herein, a polynucleotide disclosed herein, an expression vector disclosed herein, or an rAAV disclosed herein.

In an aspect, provided herein is a method for reducing the level of aggregates of a protein in a cell, the method comprising introducing into the cell a chimeric molecule disclosed herein, a pharmaceutical composition disclosed herein, a polynucleotide disclosed herein, an expression vector disclosed herein, or an rAAV disclosed herein. In an embodiment, the protein is intracellular. In an embodiment, the protein is extracellular.

In an aspect, provided herein is a method for reducing the level of human TDP-43 in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of human huntingtin in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of human phosphorylated TDP-43 in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of exon 1 of human huntingtin in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of polyglutamine (PolyQ) protein, alpha-synuclein, tau protein, amyloid beta, lamin, or phospholamban (PLN) protein in a cell, the method comprising expressing in the cell a chimeric molecule disclosed herein, a polynucleotide disclosed herein, an expression vector disclosed herein, or an rAAV disclosed herein.

In an aspect, provided herein is a method for reducing the level of aggregates of a protein in a cell, the method comprising expressing in the cell a chimeric molecule disclosed herein, a polynucleotide disclosed herein, an expression vector disclosed herein, or an rAAV disclosed herein. In an embodiment, the protein is intracellular. In an embodiment, the protein is extracellular.

In an embodiment, the cell is a muscle cell, microglia, astrocyte, neuron, or cardiomyocyte.

In an embodiment, the protein is human TDP-43, human huntingtin polyglutamine (PolyQ) protein, alpha-synuclein, tau protein, amyloid beta, lamin, phospholamban (PLN) protein. In an embodiment, the protein is phosphorylated TDP-43.

In an aspect, provided herein is a method of treating a neurodegenerative or a neuromuscular disease or disorder, the method comprising administering to a subject in need thereof an effective amount of a chimeric molecule disclosed herein, a pharmaceutical composition disclosed herein, a polynucleotide disclosed herein, an expression vector disclosed herein, or an rAAV disclosed herein.

In an aspect, provided herein is a method of treating a neurodegenerative or a neuromuscular disease or disorder, the method comprising administering to a subject in need thereof an effective amount of a polynucleotide disclosed herein, or an expression vector disclosed herein in a delivery vehicle. In an embodiment, the delivery vehicle is a lipid nanoparticle (LNP), a vesicle, an exosome, a liposome, or a polymer.

In an embodiment, the neurodegenerative disease or disorder is selected from the group consisting of motor neuron disease (MND), amyotrophic lateral sclerosis (ALS), parkinsonism syndrome, Alzheimer's dementia, progressive supranuclear palsy (PSP), Huntington's disease, multiple system atrophy (MSA), spinocerebellar ataxia (SCA1, 2, 3, 6, 7, 17), spinal-bulbar muscular atrophy (SBMA), Dentatorubral-pallidoluysian atrophy (DRPLA), and Friedreich's ataxia.

In an embodiment, the neuromuscular disease or disorder is selected from the group consisting of myopathy, hereditary cardiomyopathy, metabolic myopathy, distal myopathy, muscular dystrophy, congenital myopathy, spinal muscular atrophy (SMA), motor neuron disease, congenital myopathy, congenital muscular dystrophy, motor neuron disease, Duchenne muscular dystrophy, Becker muscular dystrophy, limb-girdle muscular dystrophies, myotonic dystrophy, myotubular myopathy, centronuclear myopathy, nemaline myopathy, selenoprotein N-related myopathy, Pompe disease, glycogen storage disease III, spinal muscular atrophy, amyotrophic lateral sclerosis, Charcot-Marie-Tooth disease, multiple sclerosis, myositis, polymyositis, and dermatomyositis.

In an embodiment, the rAAV is administered to the subject intravenously, intraperitoneally, subcutaneously, intramuscularly, intrathecally, intranasally, intracisternal, intracranially, or intradermally.

A degron comprising an amino acid sequence selected from the group consisting of SEQ ID NOs: 1-5.

In an aspect, provided herein is a chimeric molecule comprising a degron that comprises an amino acid sequence selected from the group consisting of SEQ ID NOs: 1-5. In an embodiment, the amino acid sequence of the degron consists of an amino acid sequence selected from the group consisting of SEQ ID NOs: 1-5.

### Adeno-Associated Virus Packaging Systems

In an aspect, provided herein is a packaging system for recombinant preparation of a recombinant adeno-associated virus (rAAV) disclosed herein. Such packaging systems generally comprise: a first nucleotide sequence encoding one or more AAV Rep proteins; a second nucleotide sequence encoding an AAV capsid protein; and a third nucleotide sequence comprising any of AAV genome sequence disclosed herein, wherein the packaging system is operative in a cell for enclosing the AAV genome in the capsid to form the rAAV.

In an embodiment, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In an embodiment of the packaging system, the Rep nucleotide sequence encodes an AAV2 Rep protein. Suitable AAV2 Rep proteins may include, without limitation, Rep 78/68 or Rep 68/52.

In an embodiment, the packaging system further comprises a fourth nucleotide sequence comprising one or more helper virus genes. In an embodiment, the fourth nucleotide sequence comprises adenoviral E2, E4, and VA genes. In an embodiment, the packaging system further comprises a third vector (e.g., a helper virus vector), comprising the fourth nucleotide sequence. The third vector may be an independent third vector, integral with the first vector, or integral with the second vector.

In an embodiment of the packaging system, the helper virus is selected from the group consisting of adenovirus, herpes virus (including herpes simplex virus (HSV)), poxvirus (such as vaccinia virus), cytomegalovirus (CMV), and baculovirus. In an embodiment where the helper virus is adenovirus, the adenovirus genome comprises one or more adenovirus RNA genes selected from the group consisting of E1, E2, E4, and VA. In an embodiment, the adenovirus genome comprises one or more adenovirus RNA genes selected from the group consisting of E2, E4, and VA. In an embodiment where the helper virus is HSV, the HSV genome comprises one or more HSV genes selected from the group consisting of UL5/8/52, ICPO, ICP4, ICP22, and UL30/UL42.

In an embodiment of the packaging system, the first, second, and/or third vector are contained within one or more plasmids. In an embodiment, the first vector and the third vector are contained within a first plasmid. In an embodiment, the second vector and the third vector are contained within a second plasmid.

In an embodiment of the packaging system, the first, second, and/or third vector are contained within one or more recombinant helper viruses. In an embodiment, the first vector and the third vector are contained within a recombinant helper virus. In an embodiment, the second vector and the third vector are contained within a recombinant helper virus.

In an aspect, provided herein is a method for recombinant preparation of an rAAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g., with one or more recombinant helper viruses, such as adenovirus, poxvirus (such as vaccinia virus), herpes virus (including HSV, cytomegalovirus, or baculovirus), containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins, and with a transfer genome as described herein being delivered in the form of a plasmid or a recombinant helper virus).

Accordingly, the instant disclosure provides a packaging system for preparation of an rAAV, wherein the packaging system comprises: a first nucleotide sequence encoding one or more AAV Rep proteins; a second nucleotide sequence encoding an AAV capsid protein disclosed herein; a third nucleotide sequence comprising an rAAV genome sequence described herein; and optionally a fourth nucleotide sequence comprising one or more helper virus genes (e.g., adenoviral E2, E4, and VA genes).

### EXAMPLES

### Example 1: Analysis of TDP-43 scFvs

A mutant form of TDP-43 leads to cytosolic aggregates, which act as inclusion bodies in patients with amyotrophic lateral sclerosis (ALS) and frontotemporal lobar degeneration (FTLD). Targeting the mutant form of TDP-43 can restore the balance between the ratio of wild-type TDP-43 and pathological, aggregated TDP-43.

A panel of TDP-43 scFvs were analyzed for their ability to reduce the level of TDP-43 aggregates in U2OS cells. The TDP-43 scFvs used in this example are listed in Table 10 below.

TABLE 10
Epitope specificity of TDP-43 scFvs

| TDP43 | Epitope position |
|---|---|
| scFv | on TDP-43 amino |
| clone | acid sequence |
| #050 | 104-176 |
| #051 | 247 |
| #052 | 201-211 |
| #053 | 121-127 |
| #054 | 213-223 |
| #055 | 381-391 |
| #056 | 133-139 |
| #057 | 9-15 |
| #058 | 409-410 |
| #059 | 409-410 |
| #060 | 409-410 |
| #061 | 317-343 |
| #062 | 115-118 |
| #063 | 65-71 |
| #064 | 215-222 |
| #093 | 397-411 |
| #094 | 397-411 |
| #095 | 140-200 |
| #096 | 352-366 |
| #097 | 199-213 |
| #098 | 389-411 |
| #099 | 181-195 |
| #100 | 199-213 |
| #101 | 307-321 |
| #102 | 199-213 |
| #103 | 199-213 |

U2OS cells were transfected for 48 hours with TDP-43-ANLS2KQ-GFP and either an anti-TDP-43 scFv (clone #s 50-64 and 93-103, see Tables 3-5 for sequences) or a non-TDP43 scFv (clone #s 65-66) in a 24 well plate. The cells were subsequently fixed with 4% formaldehyde in aqueous solution and stained with DAPI and mouse-a-FLAG then goat-a-mouse 568. Samples were then imaged using an ImageXpress PICO system.

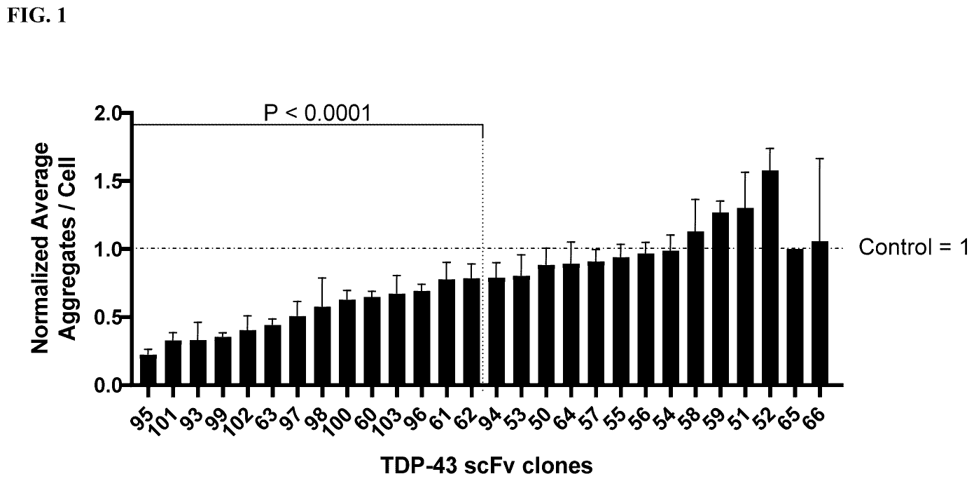

The TDP-43 aggregates were quantified using the puncta scoring module which is present in the cell reporter express software. The level of TDP-43 aggregates per cell were normalized to the control cells (transfected with clone #65). The results shown in FIG. 1 show that several of the anti-TDP-43 scFvs caused a decrease in the level of TDP-43 aggregates per cell, compared to the cells transfected with a non-TDP-43 scFv. Clone 51 does not show a reduction in TDP-43 aggregates and thus, was selected for designing scFv-degrons to determine the effect of each degron on TDP-43 aggregates.

### Example 2: Design of Chimeric Degron-scFv Molecules

Chimeric degron-scFv constructs were developed with a degron that directs protein degradation through either the ubiquitin proteasome system (UPA), chaperone mediated autophagy (CMA), or macroautophagy linked to an anti-TDP-43 scFv. Specifically, the degrons disclosed in Table 1 were linked to either the N-terminus or C-terminus of the anti-TDP-43 scFv (clone #51). The details of each degron-scFv construct are described in Table 11 below. Linker sequences GSGSGSS (SEQ ID NO: 22) and GSGSS (SEQ ID NO: 23) were used to link the degron to the scFv. The constructs also included a FLAG tag for experimental analyses.

TABLE 11
Degron-TDP-43 scFv constructs

| | | | Protein |
|---|---|---|---|
| Construct | | Position | degradation |
| # | Degron | of degron | pathway |
| 104 | 1 (SEQ ID NO: 19) | C-term | UPS |
| 105 | 2 (SEQ ID NO: 9) | C-term | UPS |
| 106 | 3 (SEQ ID NO: 20) | C-term | UPS |
| 107 | 4 (SEQ ID NO: 10) | C-term | UPS |
| 108 | 5 (SEQ ID NO: 11) | N-term | UPS |
| 109 | 6 (SEQ ID NO: 12) | C-term | UPS |
| 110 | 7 (SEQ ID NO: 13) | C-term | UPS |
| 111 | 8 (SEQ ID NO: 14) | C-term | UPS |
| 112 | 9 (SEQ ID NO: 6) | C-term | UPS |
| 113 | 10 (SEQ ID NO: 7) | C-term | UPS |
| 114 | 11 (SEQ ID NO: 18) | N-term | UPS |
| 115 | 12 (SEQ ID NO: 15) | C-term | UPS |
| 116 | 13 (SEQ ID NO: 16) | N-term | UPS |
| 117 | 14 (SEQ ID NO: 21) | C-term | CMA |
| 118 | 15 (SEQ ID NO: 8) | C-term | CMA |
| 119 | 16 (SEQ ID NO: 17) | C-term | CMA |
| 120 | 17 (SEQ ID NO: 1) | C-term | CMA |
| 121 | 18 (SEQ ID NO: 2) | N-term | macroautophagy |
| 122 | 19 (SEQ ID NO: 3) | C-term | macroautophagy |
| 123 | 20 (SEQ ID NO: 4) | N-term | macroautophagy |
| 124 | 21 (SEQ ID NO: 5) | C-term | macroautophagy |

### Example 3: Analysis of Aggregate Reduction by Chimeric Degron-scFv Constructs

A panel of chimeric degron-TDP-43-scFv molecules were analyzed for their ability to reduce TDP-43 aggregates in U2OS cells.

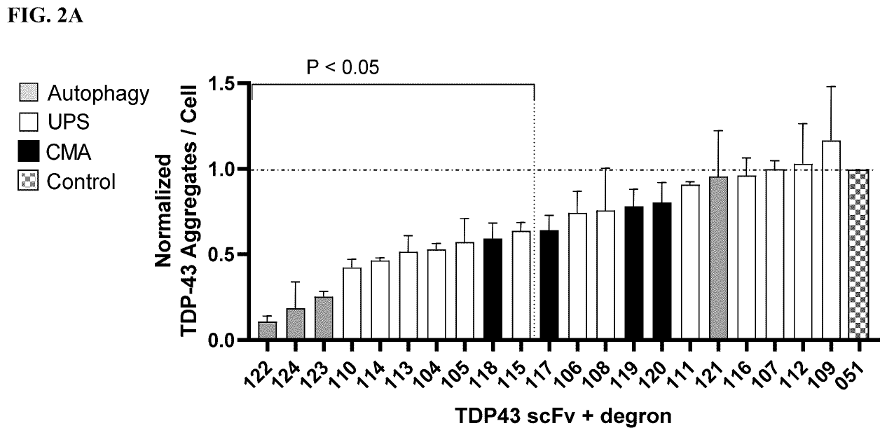

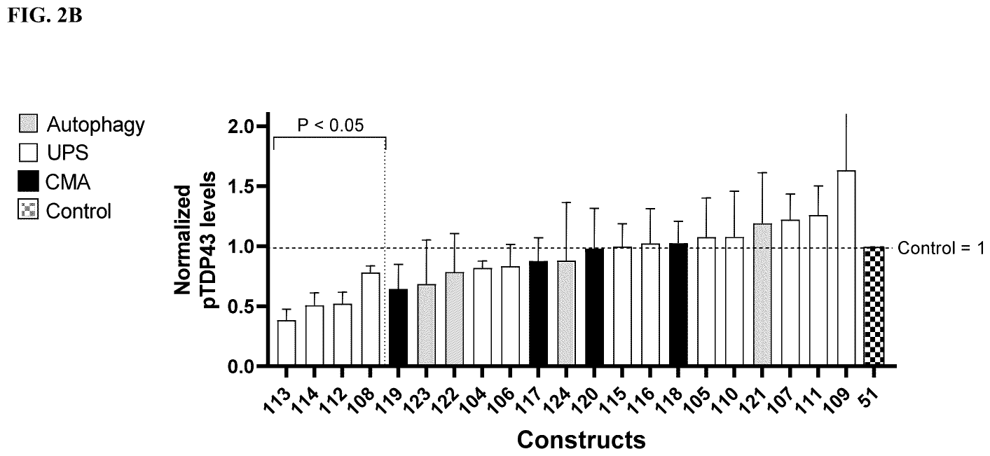

To analyze the effects of the degron constructs on TDP-43 aggregates, the TDP43-ANLS2KQ-GFP construct was co-expressed in U2OS cells with either a TDP-43 scFv+degron constructs (#104-124) or an scFv backbone construct (#51) with no degron, for 48 hours in a 24 well plate. The samples were then imaged using an ImageXpress PICO system. The results in FIG. 2A show that degron-TDP-43 scFv construct #s 104, 105, 110, 113-115, 118, and 122-124 significantly reduce the level of TDP-43 aggregates compared to the control construct.

The effects of the degron-scFv constructs on phosphorylated TDP-43 levels were assessed by western blot and densitometry analysis. U2OS cells stably expressing TDP-43 under an IPTG promotor were transfected with the degron-scFv constructs (#s 104-124), or an empty scFv backbone (clone #51) in a 24 well plate. Samples were lysed 24 hours after a 2-hour treatment with 250 μM of NaAsO2 and subjected to western blot analysis. Western blots were stained for rat-α-pTDP-43 followed by detection with goat-a-rat-800. For loading controls, the blots were stained with rabbit-α-TUBB3 followed by detection with goat-a-rabbit 680. The results in FIG. 2B show that degron-TDP-43 scFv construct #s 108 and 112-114 significantly reduce the level of phosphorylated TDP-43 aggregates compared to the control construct.

### Example 4: Analysis of Specific Protein Degradation Pathways

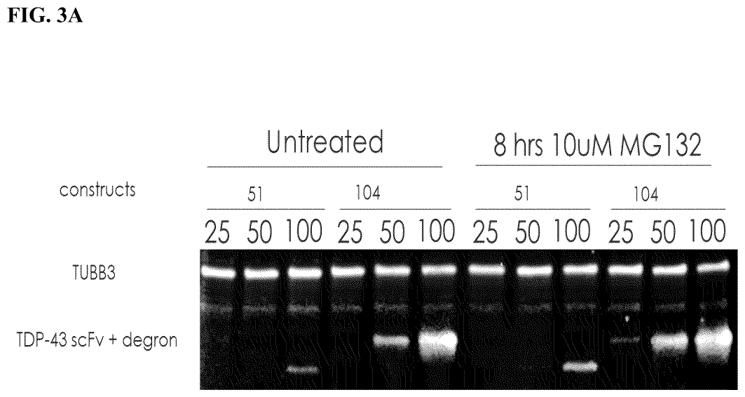

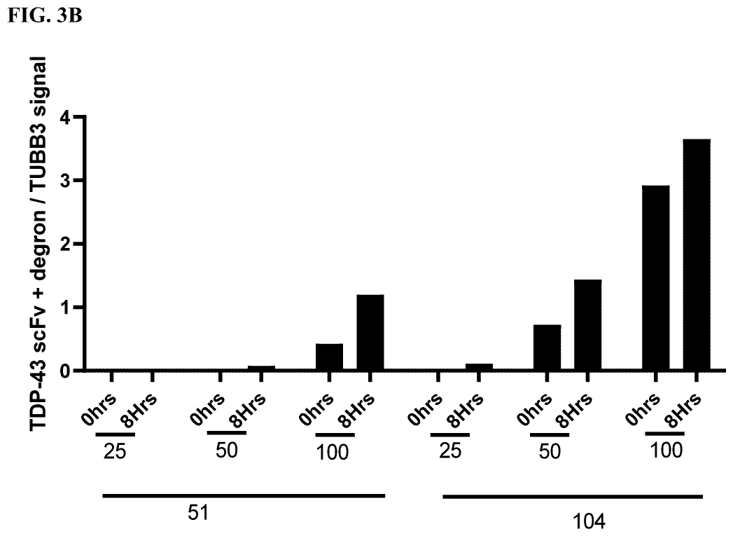

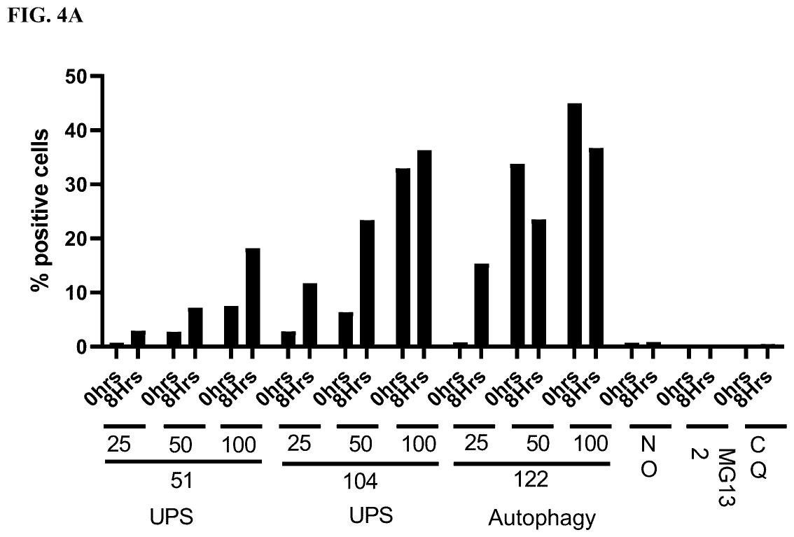

The specific protein degradation effects of the degron-scFv constructs were analyzed treating cells with proteasome and autophagy inhibitors. Specifically, U2OS cells were transfected with 25, 50, or 100 ng of the degron-scFv construct #104 or the scFv construct with no degron #51 for 24 hours in a 24 well plate. Following transfection, the cells were treated with 10 μM of the proteasome inhibitor MG132 for 8 hours or left untreated and then lysed. Western blots were stained with mouse-a-FLAG followed by detection with goat-a-mouse-800 (TDP-43 scFv+degron) and rabbit-α-TUBB3 followed by detection with goat-a-rabbit 680 (loading control). The results in FIG. 3A and FIG. 3B show that the degron TDP-43 scFv construct #104 accumulates when the cells are treated with MG132, indicating that construct #104 causes degradation through the ubiquitin proteasome system.

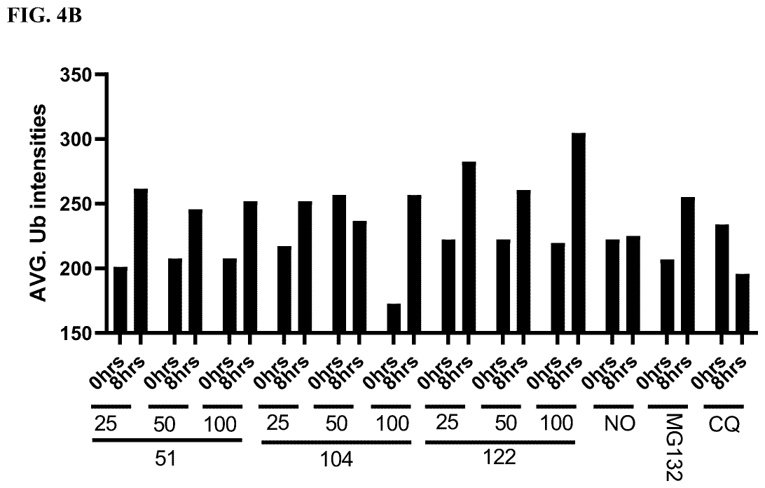

U2OS cells were transfected with either 25, 50, of 100 ng of degron-TDP-43 scFv constructs (#104 or 122), an empty scFv backbone with no degron (#51) for 24 hours in a 24 well plate. Un-transfected cells were used as a control. The cells were then treated for either 8 hours with 10 μM of the proteasome inhibitor MG132 or 8 hours with 10 μM of the autophagy inhibitor Chloroquine (CQ). Untreated cells were used as a control. Samples were subsequently fixed and stained using DAPI and mouse-a-FLAG then goat-a-mouse 568. Samples were also fixed and stained with DAPI and rabbit-α-ubiquitin then goat-a-rabbit 647 to analyze ubiquitin levels. Samples were then imaged using an ImageXpress PICO system. Images were quantified using a cell scoring module which is present in the cell reporter express software by Molecular Devices. The results in FIG. 4A show that the degron-scFv construct #104 accumulates after treatment with MG132 and the degron-scFv construct #122 accumulates after treatment with CQ, showing that these degron constructs cause degradation through the ubiquitin proteasome system and autophagy, respectively. The results in FIG. 4B show that ubiquitin levels increased following treatment with MG132.

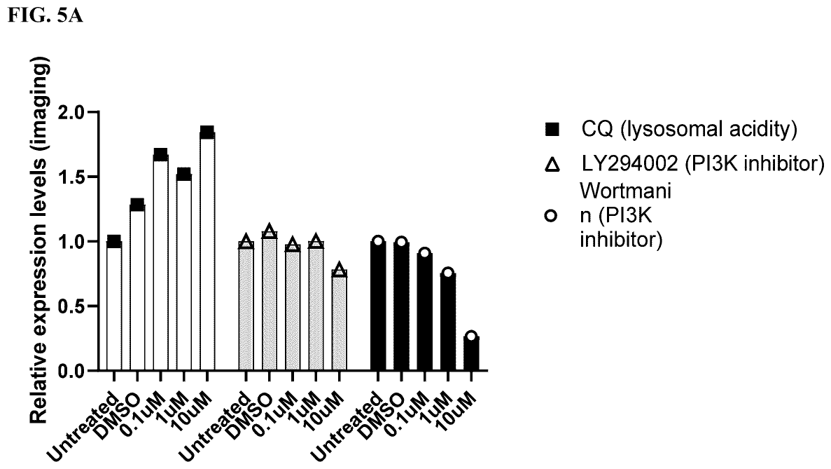

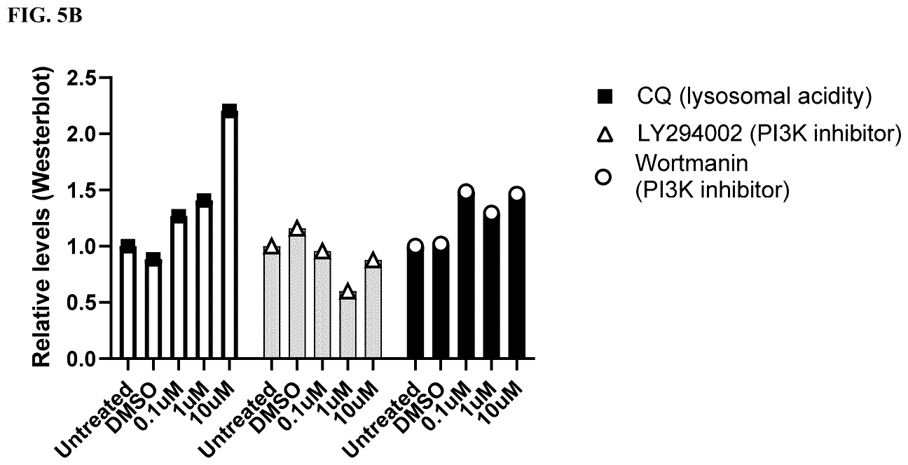

To further analyze the degrons that cause degradation through macroautophagy, U2OS cells were transfected with 50 ng of the degron-TDP-43-scFv construct #121 or 122 for 24 hours in a 24 well plate. The cells were then treated for 8 hours with 0.1, 1, or 10 μM of autophagy inhibitor chloroquine (CQ), the PI3K inhibitor LY294002, Wortmannin, or DMSO. Untreated samples were used as controls. Samples were subsequently fixed and stained with DAPI and mouse-a-FLAG then goat-a-mouse 568. Samples were then imaged using an ImageXpress PICO system. Images were quantified using a cell scoring module which is present in the cell reporter express software by Molecular Devices. Western blots were stained with mouse-a-FLAG followed by detection with goat-a-mouse-800 (TDP-43 scFv+degron) and rabbit-α-TUBB3 followed by detection with goat-a-rabbit 680 (loading control). The results in FIG. 5A and FIG. 5B show that the degron-TDP-43-scFv constructs #121 and 122 accumulate following treatment with CQ, indicating that these degron constructs cause degradation autophagy.

### Example 5: Analysis of Huntingtin (HTT) Aggregate or Soluble HTT Protein Reduction by Chimeric Degron-scFv Constructs

Chimeric degron-scFv constructs were developed with a degron that directs protein degradation through either the ubiquitin proteasome system (UPS), chaperone mediated autophagy (CMA), or macroautophagy, linked to an anti-HTT scFv. Specifically, the degrons disclosed in Table 1 were linked to either the N-terminus or C-terminus of the anti-HTT scFv (clone #252 or #240). The details of each degron-scFv construct are described in Tables 12-13 below. Linker sequences GSGSGSS (SEQ ID NO: 22) and GSGSS (SEQ ID NO: 23) were used to link the degron to the scFv. The constructs also included a FLAG tag for experimental analyses.

TABLE 12
Degron-HTT INT41 scFv constructs

| | | | Protein |
|---|---|---|---|
| Construct | | Position | degradation |
| # | Degron | of degron | pathway |
| 252 (INT41) | N.A. | N.A | N.A. |
| 354 | 9 (SEQ ID NO: 6) | C-term | UPS |
| 346 | 1 (SEQ ID NO: 19) | C-term | UPS |
| 347 | 2 (SEQ ID NO: 9) | C-term | UPS |
| 348 | 3 (SEQ ID NO: 20) | C-term | UPS |
| 349 | 4 (SEQ ID NO: 10) | C-term | UPS |
| 350 | 5 (SEQ ID NO: 11) | N-term | UPS |
| 352 | 7 (SEQ ID NO: 13) | C-term | UPS |
| 353 | 8 (SEQ ID NO: 14) | C-term | UPS |
| 355 | 10 (SEQ ID NO: 7) | C-term | UPS |
| 356 | 11 (SEQ ID NO: 18) | N-term | UPS |
| 357 | 12 (SEQ ID NO: 15) | C-term | UPS |
| 358 | 13 (SEQ ID NO: 16) | N-term | UPS |
| 359 | 14 (SEQ ID NO: 21) | C-term | CMA |
| 360 | 15 (SEQ ID NO: 8) | C-term | CMA |
| 361 | 16 (SEQ ID NO: 17) | C-term | CMA |
| 362 | 17 (SEQ ID NO: 1) | C-term | CMA |
| 363 | 18 (SEQ ID NO: 2) | N-term | macroautophagy |
| 364 | 19 (SEQ ID NO: 3) | C-term | macroautophagy |
| 365 | 20 (SEQ ID NO: 4) | N-term | macroautophagy |
| 366 | 21 (SEQ ID NO: 5) | C-term | macroautophagy |
| 351 | 6 (SEQ ID NO: 12) | C-term | UPS |
| Ctrl | N.A. | N.A | N.A. |

TABLE 13
Degron-HTT C4 scFv constructs

| | | | Protein |
|---|---|---|---|
| Construct | | Position | degradation |
| # | Degron | of degron | pathway |
| 519 | 12 (SEQ ID NO: 15) | C-term | UPS |
| 240 (C4) | N.A. | N.A. | N.A. |
| 391 | 1 (SEQ ID NO: 19) | C-term | UPS |
| 509 | 2 (SEQ ID NO: 9) | C-term | UPS |
| 510 | 3 (SEQ ID NO: 20) | C-term | UPS |
| 511 | 4 (SEQ ID NO: 10) | C-term | UPS |
| 512 | 5 (SEQ ID NO: 11) | N-term | UPS |
| 513 | 6 (SEQ ID NO: 12) | C-term | UPS |
| 514 | 7 (SEQ ID NO: 13) | C-term | UPS |
| 515 | 8 (SEQ ID NO: 14) | C-term | UPS |
| 516 | 9 (SEQ ID NO: 6) | C-term | UPS |
| 517 | 10 (SEQ ID NO: 7) | C-term | UPS |
| 518 | 11 (SEQ ID NO: 18) | N-term | UPS |
| 520 | 13 (SEQ ID NO: 16) | N-term | UPS |
| 521 | 14 (SEQ ID NO: 21) | C-term | CMA |
| 522 | 15 (SEQ ID NO: 8) | C-term | CMA |
| 523 | 16 (SEQ ID NO: 17) | C-term | CMA |
| 524 | 17 (SEQ ID NO: 1) | C-term | CMA |
| 525 | 18 (SEQ ID NO: 2) | N-term | macroautophagy |
| 526 | 19 (SEQ ID NO: 3) | C-term | macroautophagy |
| 527 | 20 (SEQ ID NO: 4) | N-term | macroautophagy |
| 528 | 21 (SEQ ID NO: 5) | C-term | macroautophagy |
| Ctrl | N.A. | N.A. | N.A. |

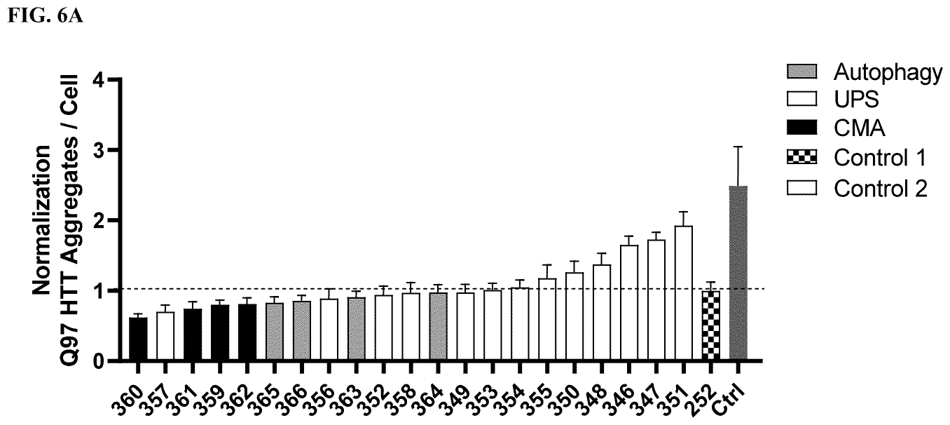

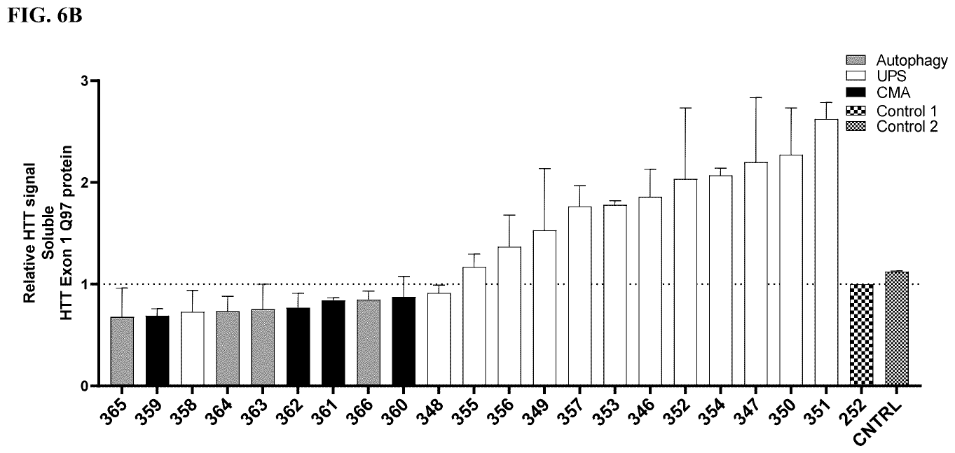

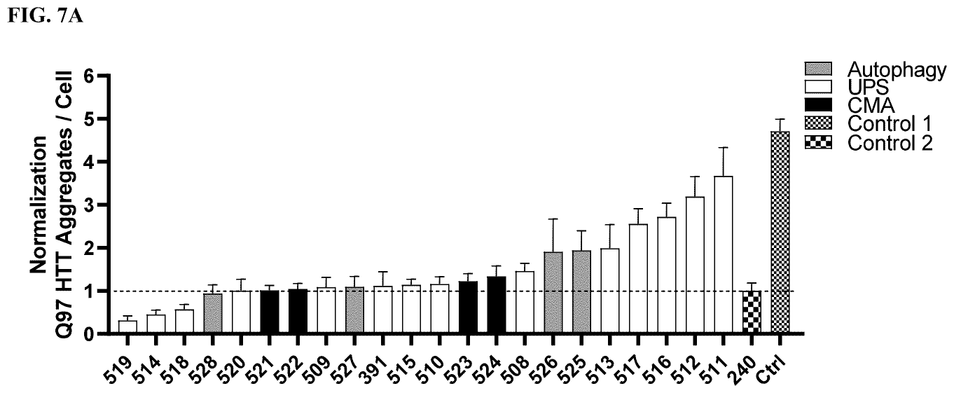

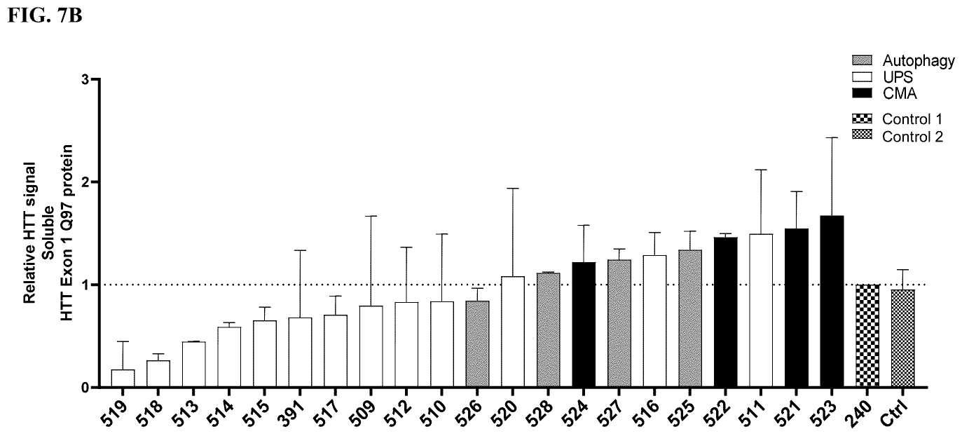

A panel of chimeric degron-HTT scFv molecules were analysed for their ability to reduce HTT aggregates or soluble HTT in U2OS cells. To analyze the effects of the degron constructs on HTT aggregates, the HTTexon1-Q97-IRES-GFP-Q16 construct was co-expressed in U2OS cells with either a HTT scFv+degron constructs or an scFv backbone construct with no degron, for 48 hours in a 24 well plate. The samples were then imaged using an ImageXpress Micro-confocal system. The results in FIG. 6A show improved Q97 HTT aggregate reduction by several degron-HTT scFv construct #s compared to the control constructs with either the HTT scFv backbone construct with no degron (Clone #252) or a non-HTT binding anti-cocaine control scFv (Ctrl). Similarly, FIG. 7A shows improved Q97 HTT aggregate reduction by several degron-HTT scFv construct #s compared to the control constructs with either the HTT scFv backbone construct with no degron (Clone #240) or a non-HTT binding anti-cocaine control scFv (Ctrl).

The panel of chimeric degron-HTT scFv molecules were further analyzed for their ability to reduce soluble exon 1 HTT protein in U2OS cells. To analyze the effects of the degron constructs on HTT aggregates, the HTTexon1-Q97-IRES-GFP-Q16 construct was co-expressed in U2OS cells with either HTT scFv+degron constructs or an scFv backbone construct with no degron, for 48 hours in a 24 well plate. The samples were then lysed and analyzed via western blot and imaged using a ChemiDoc system. FIG. 6B shows improved soluble HTT exon 1 Q97 protein reduction by several degron-HTT scFv construct #s compared to the control constructs with either the HTT scFv backbone construct with no degron (Clone #252) or a non-HTT binding anti-cocaine control scFv (Ctrl). FIG. 7B shows improved soluble HTT exon 1 Q97 protein reduction by several degron-HTT scFv construct #s compared to the control constructs with either the HTT scFv backbone construct with no degron (Clone #240) or a non-HTT binding anti-cocaine control scFv (Ctrl).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A chimeric molecule comprising a degron and a binding moiety, wherein the degron comprises an amino acid sequence selected from the group consisting of SEQ ID NOs: 1-21.

2-10. (canceled)

11. The chimeric molecule of claim 1, wherein the binding moiety is an antibody.

12. The chimeric molecule of claim 11, wherein the antibody is an scFv, heavy chain only antibody, variable domain of new antigen receptor (VNAR), antibody fragment, antigen binding (Fab) fragment, monobody, DARPin, VHH antibody, or nanobody.

13-22. (canceled)

23. The chimeric molecule of claim 1, wherein the chimeric molecule comprises from N-terminus to C-terminus:
a) a degron comprising an amino acid sequence selected from the group consisting of SEQ ID NOs: 1-8;
b) a peptide linker; and
c) an antibody.

24-28. (canceled)

29. The chimeric molecule of claim 12, wherein the antibody is an scFv.

30. (canceled)

31. The chimeric molecule of claim 29, wherein the degron comprises an amino acid sequence selected from the group consisting of SEQ ID NOs: 1-19.

32. The chimeric molecule of claim 31, wherein the scFv comprises a heavy chain variable domain and a light chain variable domain comprising the CDRH1, CDRH2, CDRH3, CDRL1, CDRL2, and CDRL3 amino acid sequences of SEQ ID NOs: 34, 35, 36, 37, 38, and 39; 40, 41, 42, 43, 44, and 45; 46, 47, 48, 49, 50, and 51; 52, 53, 54, 55, 56, and 57; 58, 59, 60, 61, 62, and 63; 64, 65, 66, 67, 68, and 69; 70, 71, 72, 73, 74, and 75; 76, 77, 78, 79, 80, and 81; 82, 83, 84, 85, 86, and 87; 88, 89, 90, 91, 92, and 93; 94, 95, 96, 97, 98, and 99; 100, 101, 102, 103, 104, and 105; 106, 107, 108, 109, 110, and 111; 112, 113, 114, 115, 116, and 117; 118, 119, 120, 121, 122, and 123; 124, 125, 126, 127, 128, and 129; 130, 131, 132, 133, 134, and 135; 136, 137, 138, 139, 140, and 141; 142, 143, 144, 145, 146, and 147; 148, 149, 150, 151, 152, and 153; 154, 155, 156, 157, 158, and 159; 160, 161, 162, 163, 164, and 165; 166, 167, 168, 169, 170, and 171; 172, 173, 174, 175, 176, and 177; 178, 179, 180, 181, 182, and 183; 184, 185, 186, 187, 188, and 189; 190, 191, 192, 193, 194, and 195; or 196, 197, 198, 199, 200, and 201, respectively.

33. The chimeric molecule of claim 32, wherein the heavy chain variable domain and light chain variable domain comprise the amino acid sequences of SEQ ID NOs: 202 and 230; 203 and 231; 204 and 232; 205 and 233; 206 and 234; 207 and 235; 208 and 236; 209 and 237; 210 and 238; 211 and 239; 212 and 240; 213 and 241; 214 and 242; 215 and 243; 216 and 244; 217 and 245; 218 and 246; 219 and 247; 220 and 248; 221 and 249; 222 and 250; 223 and 251; 224 and 252; 225 and 253; 226 and 254; 227 and 255; 228 and 256; or 229 and 257, respectively.

34-35. (canceled)

36. The chimeric molecule of claim 12, wherein the scFv comprises a heavy chain variable domain and a light chain variable domain comprising the CDRH1, CDRH2, CDRH3, CDRL1, CDRL2, and CDRL3 amino acid sequences of SEQ ID NOs: 292, 293, 294, 295, 296, and 297; 301, 302, 303, 304, 305, and 306; 307, 308, 309, 310, 311, and 312; 313, 314, 315, 316, 317, and 318; 319, 320, 321, 322, 323, and 324; 325, 326, 327, 328, 329, and 330; 331, 332, 333, 334, 335, and 336; 343, 344, 345, 346, 347, and 348; 352, 353, 354, 355, 356, and 357; 358, 359, 360, 361, 362, and 363; 364, 365, 366, 367, 368, and 369; 370, 371, 372, 373, 374, and 375; 376, 377, 378, 379, 380, and 381; or 382, 383, 384, 385, 386, and 387.

37. The chimeric molecule of claim 36, wherein the heavy chain variable domain and light chain variable domain comprise the amino acid sequences of SEQ ID NOs: 388 and 404; 389 and 406; 390 and 407; 391 and 408; 392 and 409; 393 and 410; 394 and 411; 397 and 412; 398 and 414; or 399 and 415.

38-40. (canceled)

41. The chimeric molecule of claim 29, wherein the scFv specifically binds to human polyglutamine (polyQ) protein, human TDP-43, human huntingtin, human alpha-synuclein, human tau protein, human amyloid beta, human lamin, or human phospholamban (PLN) protein).

42-47. (canceled)

48. A polynucleotide encoding the chimeric molecule of claim 11.

49-51. (canceled)

52. An expression vector comprising the polynucleotide of claim 48.

53. A host cell comprising the polynucleotide of claim 48.

54. A method of producing a chimeric molecule, the method comprising culturing the host cell of claim 53 under conditions such that the polynucleotide is expressed and the chimeric molecule is produced.

55. A recombinant adeno associated virus (rAAV) comprising a capsid and a viral genome, wherein the viral genome comprises at least one inverted terminal repeat (ITR) region and a polynucleotide encoding the chimeric molecule of claim 11.

56-59. (canceled)

60. A method for reducing the level of aggregates of a protein in a cell, the method comprising introducing into the cell the chimeric molecule of claim 1.

61-64. (canceled)

65. A method of treating a neurodegenerative or a neuromuscular disease or disorder, the method comprising administering to a subject in need thereof an effective amount of the chimeric molecule of any of claim 1.

66-71. (canceled)

72. A chimeric molecule comprising a degron that comprises an amino acid sequence selected from the group consisting of SEQ ID NOs: 1-5.

73. (canceled)

74. A polynucleotide encoding the chimeric molecule of claim 72.

75-77. (canceled)

78. An expression vector comprising the polynucleotide of claim 74.

79. A host cell comprising the polynucleotide of claim 74.

80. A method of producing a chimeric molecule, the method comprising culturing the host cell of claim 79 under conditions such that the polynucleotide is expressed and the chimeric molecule is produced.

81. A recombinant adeno associated virus (rAAV) comprising a capsid and a viral genome, wherein the viral genome comprises at least one inverted terminal repeat (ITR) region and a polynucleotide encoding the chimeric molecule of claim 72.

82. (canceled)

83. The rAAV of claim 81, wherein the capsid protein comprises the amino acid sequence of SEQ ID NOs: 258-289.

84. A pharmaceutical composition comprising the the chimeric molecule of claim 72 and a pharmaceutically acceptable carrier.
